(19) United States
(12) Patent Application Publication
(10) Pub. No.: US 2007/0172422 A1
(43) Pub. Date: Jul. 26, 2007

(54) Exponential pattern recognition based cellular targeting compositions, methods and anticancer applications

(76) Inventor: Arnold Glazier, Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/593,938

(22) Filed: Nov. 7, 2006

Related U.S. Application Data
(63) Continuation of application No. 10/179,610, filed on Jun. 24, 2002, now abandoned.
(60) Provisional application No. 60/300,805, filed on Jun. 25, 2001.

(51) Int. Cl. A61K 51/00; A61K 38/16; C07K 14/00; C07K 9/00

(52) U.S. Cl. 424/1.11; 424/178.1; 514/12; 514/15; 514/7; 514/8; 530/322

(57) ABSTRACT

The present invention relates to the compositions, methods, and applications of a new approach to pattern recognition based targeting by which an exponential amplification of effector response can be specifically obtained at a targeted cells. The purpose of this invention is to enable the selective delivery of large quantities of an array of effector molecules to target cells for diagnostic or therapeutic purposes. The invention is comprised of two components designated as “Compound 1” and “Compound 2”: Compound 1 is comprised of a cell binding agent and a masked female adaptor. Compound 2 is comprised of a male ligand, an effector agent, and two or more masked female receptors. The male ligand is selected to bind with high affinity to the female adaptor. Compound 1 can bind with high affinity to the target cell and the female receptor can then be unmasked by an enzyme enriched at the tumor cell. The male ligand of Compound 2 can then bind to the unmasked female adaptor bound to the target cell. The masked female adaptor on the bound Compound 2 can then be specifically unmasked. One receptor has in effect become two. Two new molecules of Compound 2 can bind to the unmasked adaptors receptors. After unmasking two receptors in effect become four. The process can continue in an explosive exponential like fashion resulting in enormous amplification of the number of effector molecules specifically deposited at the target cell.

### RELATED APPLICATIONS

This application is a continuation of U.S. application Ser. No. 10/179,610, filed Jun. 24, 2002, which claims the benefit of U.S. Provisional Application No. 60/300,805, filed Jun. 25, 2001. The entire teachings of the above applications are incorporated herein by reference.

### BACKGROUND OF THE INVENTION

The fundamental technical obstacle to the development of safe and effective anti-cancer drugs is the problem of tumor specificity Pattern recognition based tumor targeting or multi-factorial targeting was developed to provide a practical basis for tumor specific targeting. This technology was disclosed in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs: the contents of which are incorporated herein by reference in their entirety. Specificity in pattern recognition targeting tumor resides in the pattern comprised of a small number of normal proteins. Tumor specificity resides not in the normal proteins but in simple patterns of normal proteins that characterize the malignant phenotypes. The pattern recognition based targeting technology previously disclosed by Glazier involves non-amplified drug targeting wherein the total number of effector or toxin molecules delivered to a cell is a limited to a small multiple of the number of target receptors on the tumor cell. Pre-targeting strategies based on administering antibody-avidin conjugates, then clearing unbound antibody-avidin; and then administering a biotin-drug conjugate are well known and described in Sakahara H, Saga T. “Avidin-biotin system for delivery of diagnostic agents.” Adv Drug Deliv Rev 1999 37(1-3):89-101; which is hereby incorporated by reference in its entirety. Pretargeting approaches can enable only limited amplification. The amplification in the number of biotin-drug molecules bound is limited to the number of biotin binding sites per antibody molecule. In addition, these approaches do not enable the amplified delivery of drugs targeted to patterns of proteins.

At the present time there are no methods that enable pattern recognition cellular targeting with target pattern specific amplification of effector or drug delivery. In addition, at the present time there are no methods for the specific targeted delivery of an exponentially increasing quantity of drug to a target site.

### SUMMARY OR THE INVENTION

The present invention relates to the compositions, methods, and applications of a new approach to pattern recognition based targeting by which an exponential amplification of effector response can be specifically obtained at targeted cells. The purpose of this invention is to enable the selective delivery of large quantities of an array of effector molecules to target cells for diagnostic or therapeutic purposes. The invention relates to methods and compositions of a prodrug wherein said prodrug is a compound that can undergo biotransformation into a drug; wherein said drug gains the ability to selectively bind at least one additional molecule of the prodrug; and wherein bound prodrug can undergo biotransformation into the drug which can selectively bind additional molecules of the prodrug. In a preferred embodiment after unmasking the drug can bind two or more molecules of a prodrug. This cycle can repeat resulting in massive amplification of the quantity of prodrug specifically delivered to the target site.

The present invention also relates to a method for the site specific delivery to a target of effector molecules in vitro or in vivo; wherein said method is comprised of contacting the target with two compounds designated as Compound 1 and Compound 2; and wherein Compound 1 is comprised of at least one group that can bind to the target, and at least one masked female adaptor; and wherein Compound 2 is comprised of at least one male ligand; at least one masked female adaptor; and at least one effector group; and wherein the masked female adaptors cannot bind to the male ligands; and wherein the masked female adaptors can be unmasked spontaneously or by the action of an enzyme or other biomolecule at the target site to yield female adaptors; and wherein each female adaptor can bind to at least one male ligand; and each male adaptor can bind to at least one female adaptor; and wherein the effector group is a group that directly or indirectly exerts an activity at the target.

The present invention also relates to compounds and methods, and applications of pattern recognition (multi-factorial) targeting based on the aggregation of sets of targeted compounds on the target cell surface.

### BRIEF DESCRIPTION OF THE DRAWINGS

No drawings

### DETAILED DESCRIPTION OF THE INVENTION

Definitions:

Activity—A physical, chemical or biological response such as a pharmacologically beneficial response such as cytotoxicity, or a diagnostic effect.

Adaptor—A chemical group that acts like a receptor and can bind to a ligand.

Analog—A compound or moiety possessing significant structural similarity as to possess substantially the same function.

At a target cell—A phrase used to refer to in, on, or in the microenvironment of a target cell.

Binding Affinity—Tightness of binding between a ligand and receptor.

Bioreversibly Masked Group—A chemical group that is derivatized in a bioreversible manner. For example, an ester group can be a bioreversibly masked group for a hydroxy group. A bioreversible masking group is a chemical group that when bonded with a second group produces a bioreversibly masked group for said second group.

Bioreversible Protecting Group—A chemical group or trigger that can be modified in vivo or in vitro and wherein said modification unmasks the group that is protected.

Chemically Modify—To change the chemical property of a molecule by making one or more new chemical bonds and/or by breaking one or more chemical bonds of the molecule.

Connectivity—The sites at which chemical structures or functional groups are attached together to give a single molecule. For example, various connectivity between groups A, B, C include structures such as A-B-C, B-A-C, or A-C-B. Connectivity can be direct such as by a covalent bond between an atom of A and B or indirect such as through a covalently bonded linker.

Derivative—A compound or moiety that has been further modified or functionalized from the corresponding compound or moiety,

Drug—A compound that can exert a useful pharmacological activity or which is a biological effector agent

Effector—An agent that exerts an activity and evokes a physical, chemical or biological response such as a pharmacologically beneficial response such as cytotoxicity, or a diagnostic effect.

Effector Group—A chemical group that can function as an effector or which can give rise to an effector agent.

Enriched at the target—Present at a significantly greater concentration at the target then at a nontarget site; typically at least about two fold greater at the target.

Female Adaptor—A chemical group that binds selectively to its complementary male ligand. Also referred to as a “female receptor”;

Female Receptor—A chemical group that binds selectively to its complementary male ligand. Also referred to as a “female adaptor”.

Good Leaving Group—A chemical group that readily cleaves from the group to which it is attached. For example, a group that is easily displaced in a nucleophilic reaction, or which undergoes facile solvolysis in an SN1 type reaction.

IC50—The concentration of an inhibitor required to reduce the activity of an enzyme or process by 50%.

Inert Substituents—A chemical substituent that does not interfere with functionality to a significant degree.

Ki—IC50

Linker—A chemical group that serves to attach targeting ligands, triggers and effectors or other chemical structures together.

Lower Alkyl Group—A hydrocarbon containing about 10 or less carbon atoms which can be linear or cyclic and which can bear substituents.

Male Ligand—A chemical group or structure that can bind to a female adaptor

Masked Female Adaptor—A latent or protected female adaptor which when unmasked gains the ability bind to its complementary male ligand

Masked Group—A chemical group that is hidden or blocked, or derivatized until unmasked.

Microenvironment of the target—The volume of space around a target cell within which a drug is able to evoke its intended pharmacological activity upon the target. Alternatively, the volume encompassed by a sphere centered on a tumor cell with a radius of between about 10 to about 500 microns.

Multifactorial—A function of multiple factors or variables.

Multivalent Binding—Simultaneous binding at multiple targeting ligand—target receptor sites.

Non-selective Targeting Ligand—A chemical structure that binds to a receptor or physically associates with biomolecules that are ubiquitous or not enriched on the target compared to non-target.

Non-target—A cell, cells, tissue, or tissue type to-which it is not desired to direct effector activity. For example, if the target is a tumor then a normal tissue is a non-target.

Oligo-Peptide Nucleotide Analog—An analog of an oligo-nucleotide polymer wherein the phospodiester-sugar backbone is replaced with a structure comprised of carboxy-amide bonds.

Over-expressed—present at increased amounts.

Pharmacological activity—A physical, chemical or biological response that is evoked by a drug or effector agent such as a cytotoxicity or stimulation of the immune system or a diagnostic effect.

Prodrug—A compound that can undergo transformation spontaneously or under the action of biomolecules into a derivative drug compound with different physical, chemical, or pharmacological properties.

Selective Binding—Binding between a pair of compounds or groups that have a useful degree of specificity for each other but not for an unrelated third compound or group. For example, antigen- antibody binding.

Selective for a Target—A property is selective for a target if the presence of said property can allow the target to be distinguished from a non-target to a useful degree.

Specific for a target—A property is specific for a target if the property is unique to the target and absent from non-targets

Target—A cell, cells, tissue, or tissue type, or biomolecular component to which it is desired to direct effector activity such as tumor cells, or autoimmune lymphocytes.

Targeting Agent—A chemical structure or group of chemical structures composed of targeting ligand(s) that confer a degree of specificity towards a target. For example, a monoclonal antibody.

Targeting Ligand—A chemical structure, which binds with a degree of specificity to a targeting receptor.

Targeting Property—Any characteristic, feature, or factor, such as a targeting receptor, a triggering agent, an enzyme, or a chemical or biochemical factor that can be used to distinguish between target and non-target.

Targeting Receptor—A chemical structure at the target that binds with a useful degree of specificity to a targeting ligand.

Targeting Selectivity—The ability to evoke a greater effector activity at target compared to non-target.

Target Molecules—Biomolecules that are either target receptors or triggering agents such as a protein that binds a targeting ligand or an enzyme at the target cell which can activate a trigger and which are increased at a target compared to a non-target but not necessarily all non-targets.

Tissue of Tumor Origin—The tissue type from which a tumor originated. For example prostate tissue for prostate cancer.

Trigger—A chemical group which can undergo in vitro or in vivo chemical modification either spontaneously or by a triggering agent with the modification leading to trigger activation that modulates the pharmacological activity of the drug. A trigger can be considered as a chemical switch that upon activation gives a consistent and predictable output such as unmasking a chemical group, or liberating an effector agent.

Trigger Activation—The process of chemical modification that causes a trigger to modulate the pharmacological activity of the drug.

Triggering Factor—An enzyme, biomolecule or other agent that is able to activate a trigger, also referred to as a “triggering agent”.

Tumor Component—is a biomolecule that is present in tumor cells, on tumor cells, in the microenvironment of tumor cells, on tumor stromal cells or present in tumor bulk.

Tumor-selective Target Receptor—A target receptor that is present in increased amounts on tumor cells or in the microenvironment of tumor cells compared to that of normal cells, but not necessarily compared to all types of normal cells.

Tumor-selective Triggering Agent—A triggering agent, triggering factor, or triggering enzyme that is present in increased amounts on tumor cells, in tumor cells, or in the microenvironment of tumor cells compared to that of normal cells but not necessarily all types of normal cells.

The specific targeting of drugs is of fundamental importance in the treatment and diagnosis of many major medical conditions including: cancer; autoimmune disorders; infectious diseases; and transplant rejection. In some cases specific targeting receptors are available to serve as a basis for targeting specificity. In this situation a drug composed of a targeting ligand and an effector agent that can bind specifically to the target receptor on the surface of the target cell can be employed to localize the drug. However, if the density of target receptors on the target cell is low the delivery of sufficient effector agent to elicit the desired effect may not be possible. One approach that has been employed to amplify the signal involves the targeted delivery of an enzyme that specifically activates a prodrug. However, this approach requires that the prodrug be administered at relatively high concentrations. Nonspecific activation of the prodrug at non-target sites can severely limit targeting specificity. The present invention relates to compounds and methods that can enable effector amplification at target cells in the presence of ultra-low systemically nontoxic concentrations of the effector agent.

In many situations specific targeting receptors are unavailable. Pattern recognition based targeting or multi-factorial targeting was developed to address this situation. In pattern recognition targeting, specificity resides in the pattern rather than the individual components. The present invention relates to compounds and methods that can enable effector amplification of pattern recognition based targeting. The present invention provides a means by which enzymes that are enriched at the target cell or in the microenvironment of the target cell can contribute to the pattern that defines targeting specificity and enable effector amplification in the presence of ultra-low, systemically nontoxic concentrations of the effector agent.

The present invention relates to methods and compounds for the amplified, site specific delivery of effector molecules in vitro or in vivo wherein said method is comprised of contacting the target with two compounds designated as “Compound 1 and Compound 2”; wherein “Compound 1” is comprised of one or more groups that can bind to the target, and one or more groups designated as “female adaptors”, or one or more groups designated as “masked female adaptors” wherein a female adaptors can bind to a group referred to as a “male ligand”, and wherein Compound 2 is comprised of one or more male ligands that can bind to the female adaptors; one or more effector groups; and one or more female adaptors or one or more masked female adaptors; and wherein the masked female adaptors can be unmasked spontaneously or by the action of an enzyme or other biomolecule at the target site to yield a female adaptor, and wherein upon unmasking the group gains the ability to bind a male ligand; and wherein an effector group is a group that directly or indirectly that exerts an activity and evokes a physical, chemical or biological response such as a pharmacologically beneficial response such as cytotoxicity, or a diagnostic effect. In preferred embodiments Compound 2 has two or more masked female adaptors. In preferred embodiments Compound 2 has a greater number of masked female adaptors than male ligands. In a preferred embodiment Compound 2 has one male ligand and two masked female adaptors.

In a preferred embodiment the masking group(s) of the masked female adaptors are selected such that they can be unmasked by one or more enzymes that are enriched at the target site.

Terminology Employed

The following terminology is employed: A male ligand is designated as a group ‘M’. A female adaptor is designated as “F”. A protected or masked female adaptor is designated as “pF”. The specificity of the male ligand or female adaptor is described by additional notation in “( ).” For example,. F(x) can bind to M(x); F(y) can bind to M(y); but F(x) cannot bind to M(y).

A preferred embodiment of the present invention is comprised of two compounds:

Compound 1, is comprised of the groups:
{T and p[F(x)]_{q}} or {T and [F(x)]_{q}}
Wherein “T” is a targeting agent or a chemical group or groups that bind to the target receptor designated as “R” and wherein “pF(x)” is a masked female adaptor; and wherein the masked female adaptor is a chemical group that when unmasked gives rise to the receptor or adaptor designated as “F(x)” and wherein F(x) can bind to the ligand designated as “M(x)”; and wherein pF(x) can be unmasked spontaneously or by an enzyme or biomolecule which is enriched at the target or in the microenvironment of the target; and wherein “q” is the number of groups pF(x) or F(x) and wherein q is 1, 2, 3, 4, 5, 6, 7, 8, 9, 10, or about 10; or 10-about 50, or 50 to about 200; and wherein the groups pF(x) may differ and the groups F(x) may differ;.

And wherein Compound 2 is comprised of the groups:
{[M(x)]ₘ and [E]ₒ and [pF(x)]ₙ} or {[M(x)]ₘ and [E]ₒ and [F(x)]ₙ}
wherein the group designated as “E” is an effector agent or a group that exerts an activity and evokes a physical, chemical, or biological response such as a pharmacologically beneficial response such as cytotoxicity, or a diagnostic effect; and wherein the number of effector groups E which may differ, is designated as “o”; and wherein the number of groups pF(x) is designated as “n” and wherein the number of groups M(x) is designated as “m” and wherein the groups pF(x) may differ and the groups F(x) may differ; and wherein the groups M(x) may differ; and wherein “o” is 0,1,2,3,4,5,6,7,8,9, or 10 or about 10; and the number “m” of is 1,2,3,4,5,6,7,8,9,10 or about 10; or 10 to about 50, or 50 to about 200; and the number “n” is 1,2,3,4,5,6,7,8,9,10 or about 10 or about 10; or 10-to about 50, or 50 to about 200; and wherein the connectivity of the groups that comprise Compound 1 and Compound 2 may vary. The only requirement for the connectivity of the groups is that the function of the components remain intact.

In a preferred embodiment q=1; m=1; o=1; and n=2.

Compound 3

A preferred embodiment of the present invention is comprised of the above Compound 1, Compound 2, and a Compound 3 comprised of the structure:
T2-Ez or Ez-M(x) or the groups {T2 and Ez and M(x) }
wherein T2 is a targeting agent or a chemical group or groups that can bind to the target receptor designated as “R2” and Ez is an enzyme that can unmask pF(x) to give F(x). In a preferred embodiment T and T2 bind to different receptors on the target.

In a preferred embodiment of the above q=1; m=1; n=2; and o=1.

In preferred embodiments of Compound 1 and Compound 2 the female adaptors are all masked.

The present invention is also directed to the composition of matter comprised of Compound 1 and Compound 2 and Compound 3 individually and in combination as a mixture or as components of a kit. The present invention is also directed to the composition of matter comprised of Compound 1 and Compound 2 in combination as a mixture or as components of a kit. The present invention is also directed to the composition of matter comprised of Compound 2 and Compound 3 in combination as a mixture or as components of a kit.

Mechanism of Action

The mechanism of action is illustrated below for the case when only Compound 1 and Compound 2 are employed:

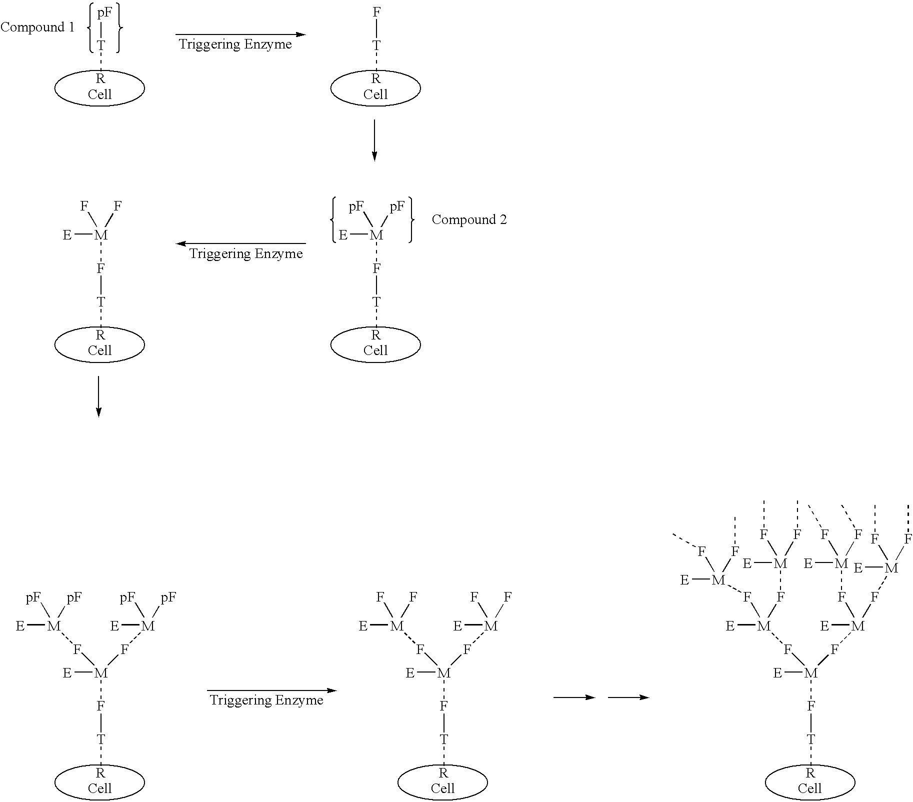

Compound 1 and Compound 2 can be administered concurrently or sequentially. Compound 1 binds to the target cell receptor “R” by the group “T”. The masked female adaptor “pF” is then unmasked by the triggering enzyme and generates the receptor “F.” A molecule of Compound 2 then binds by its group M to the receptor F. The n groups pF of the bound Compound 2 molecule are then unmasked to generate n additional female adaptors. The n adaptors in turn bind to n additional molecules of Compound 2 by the M groups. Unmasking of the adaptors on these n molecules generates an additional nˆ2 receptors. If n=1 the process can result in a linear increase of the number of effector molecules bound to the cell. If n is two or greater the number of effector molecules bound to the target can increase explosively in an exponential fashion. In principle with n=2, after only 19 cycles an effector amplification of over one million times is possible. The duration of each cycle can reflect the time required to unmask the protected receptors. Although the actual mechanism can be more complex then described above the net result can be the specific formation of large tree like aggregates containing large amounts of the effector agent specifically bound to the target. If the groups M and F possess very high mutual binding affinity than very low concentrations of the components can deliver large quantities of effector agent to the target.

If m=2, and n=2 then some additional properties can be exhibited. In this case Compound 2 can exhibit the ability to cross-link or cause higher order aggregates with molecules of Compound 1 bound to the surface of the target cell. This process is illustrated below:

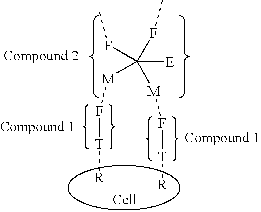

The formation of cross-links between molecules of Compound 1 on the target cell surface can dramatically increase the affinity of the complex to the target cell. The relationship between multi-site binding and increased binding affinity is well established and discussed in the following reference: Perelson, Alan S., et al., eds. Cell Surface Dynamics: Concepts and Models. New York and Basel: Marcel Dekker, Inc., 1984; which is hereby incorporated by reference in its entirety. Cross-linking between surface bound molecules should be especially efficient and rapid because of the high effective molarity of the components when confined to the two-dimensional surface of the cell membrane. Cross-linking can also occur at higher levels of the aggregate and between multiple molecules of Compound 1 bound to the cell membrane. In this case even targeting agents with relatively weak binding affinity can give very high affinity cell binding. An interesting feature of this case is that the triggering enzyme(s) that unmask the receptor F(x) can contribute to the targeting specificity at both the level of the binding of Compound 1 to the target cell and at the level of effector amplification. The mechanism of action is illustrated below for the optional case when all three components are employed:

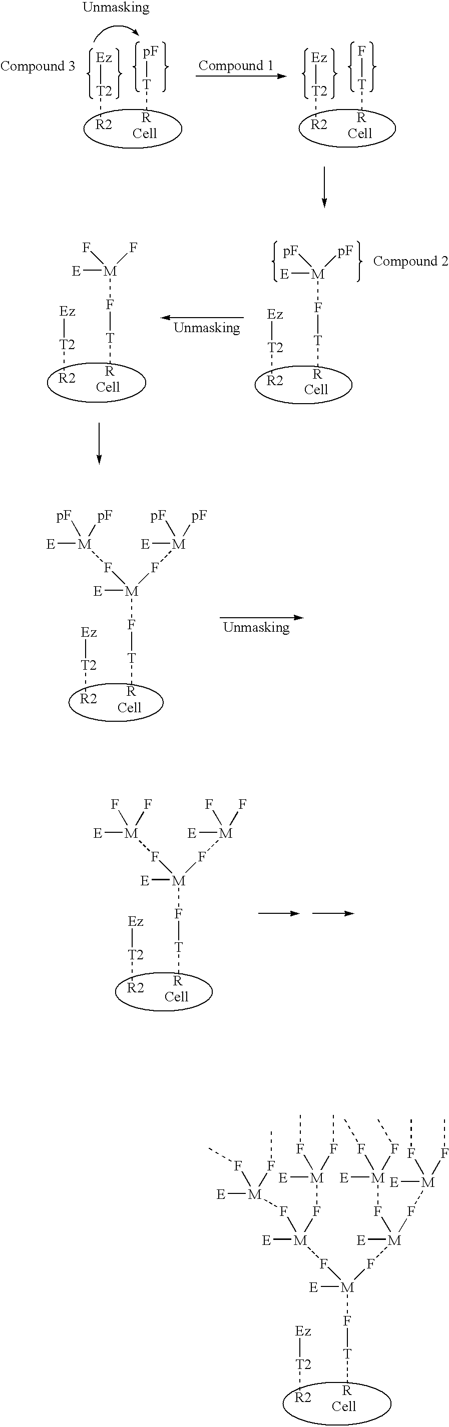

Compound 1 and Compound 3 bind to receptors “R” and “R2” respectively on the surface of the target cell. The protected female adaptor “pF” is then unmasked by the enzymatic activity of the enzyme Ez. A molecule of Compound 2 then binds to the female adaptor “F” by the male ligand “M”. The two protected female adaptors of the bound Compound 2 are then unmasked in a similar fashion by Ez. The cycle repeats ultimately depositing large quantities of the effector agent “E” at the target site. In this three-component system targeting specificity is for the pattern comprised of targets R and R2.

When Compound 3 has the structure: T2-Ez -M(x) then both Compound 2 and Compound 3 can be incorporated into the tree like aggregate that is deposited at the target producing even greater amplification.

It should be noted that if Compound 2 has the following groups:

{[M(x)]ₘ and [E]ₒ and [pF(x)]ₙ} and o is 2 or greater; and one of the effector groups E is a targeting ligand T; then this compound can be employed in the absence of Compound 1 to achieve amplified effector delivery. The mechanism of action is shown below for the case when m=1, o=2, and n=2.

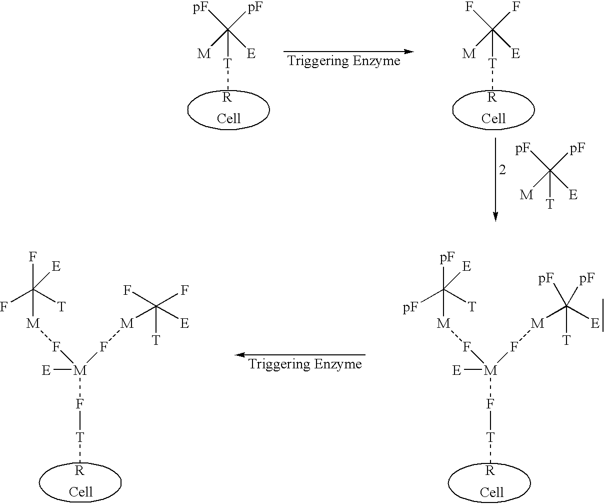

The process above can repeat and deposit large quantities of the effector agent at the target site. Compound 2 of the above structure also can cross-link the receptors R on the cell surface resulting in very high binding affinity to the target cell.

In a preferred embodiment of the present invention one of the groups E of Compound 2 is a targeting ligand or a targeting agent that can bind to the target. The present invention also includes the method comprised of contacting a target with said Compound 2. A preferred embodiment of the invention is a Compound 2 comprised of the following groups:
{[M(x)]ₘ and [E]ₒ₋₁ and [pF(x)]ₙ and T}

In a preferred embodiment m=1; o=2; n=2. In another preferred embodiment n=1 and pF(x) when unmasked can bind simultaneously to two group M(x). A preferred embodiment of this has the following structure: 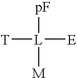
wherein L is a linker. In a preferred embodiment of the above, M is an oligonucleotide or oligonucleotide analog and pF is a complementary oligonucleotide or analog thereof that when unmasked can bind two M. In a preferred embodiment of the above the oligonucleotides are peptide nucleotide analogs.

Another preferred embodiment of the invention is comprised of a set of Compound 1; Compound 2; and a second Compound 2; wherein Compound 2 are comprised of:
{[M(x)]ₘ and [E]ₒ and [pF(y)]ₙ} or {[M(x)]ₘ and [E]ₒ and [F(y)]ₙ}
and the second Compound 2 is a comprised of:
{[M(y)]ₘ and [E]ₒ and pF(x)]ₙ} or {[M(y)]ₘ and [E]ₒ and [F(x)]ₙ}

When a target is contacted with these three components a large tree like aggregate comprised essentially of alternating types of Compound 2 anchored to the target by Compound 1 can form. In a preferred embodiment different enzymes are required to unmask pF(x) and PF(y)

In a preferred embodiment one of the effector groups in Compound 2 is comprised of an enzyme that can unmask pF(y) and one of the effector groups of the second type of Compound 2 is an enzyme that can unmask pF(x). This system by providing a means to exponentially amplify the triggering enzymes at the target site can enable massive amplification of the targeted drug delivery. In this particular embodiment targeting specificity will be defined by the initial targeting agents.

In a preferred embodiment of the present invention Compound 1 is a multi-valent delivery vehicle; designated as “ET” as described in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs”. in which the effector agent E is comprised of the group pF(x) of F(x). The only requirement for the connectivity of the groups that comprise Compound 1, Compound 2, Compound 3, is the requirement that the function of the groups remain intact. Since the receptors are not fixed in space the scope of possible connectivities that are compatible is very large. One skilled in the arts will recognize that many suitable connectivities of the different groups which are to be considered within the scope of the present invention.

In addition to the groups T, pF(x), and F(x), Compound 1 can optionally also have additional groups such as effector agents “E” and triggers that bioreversibly connect the effector agents to Compound 1.

In a preferred embodiment Compound 2 is comprised of a group F(x) and a group M(x) and the groups are connected in such as manner as to inhibit intramolecular binding between said groups or such that intramolecular binding is weaker than intermolecular binding. This can be accomplished by connecting the groups in such a manner that steric or geometric factors preclude proper or favorable alignment for binding. It should be noted that a Compound 2 comprised with groups F(x) is a metabolite derived from the corresponding compound with groups pF(x).

In an even more preferred embodiment of the present invention the linker and positioning of groups pF(x) and M(x) are selected such that intramolecular binding between the group M(x) and F(x) of Compound 2 can occur. This can increase the pattern recognition targeting specificity. For optimal amplification the following steps must occur in the following time sequence:
1. Binding of the male ligand of component two to a female adaptor attached to the target
2. Unmasking of the masked female adaptors of the bound Compound 2 by triggering enzyme at the target
3. Repetition of the above steps

If the order of step 1 and step 2 is reversed, and the mean dissociation time of F from M is long, then the chain reaction can be quenched by the intramolecular binding of the male ligand with a female adaptor in the same molecule. This will be especially the case if n=m. Targeting specificity will be for the pattern comprised of both the targeting receptor to which T binds and the triggering enzyme.

The present invention also relates to compounds and methods, and applications of pattern recognition (multi-factorial) targeting based on the aggregation of sets of components on the target cell surface. The aggregation of components at the cell surface can result in dramatically enhanced binding affinity because of the multi-valent nature of the interactions. As discussed in detail in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs” the pattern comprised of a small number of normal proteins can be highly specific for tumor cell despite the fact that no normal protein alone is tumor specific. Accordingly, methods to target patterns rather than individual components of the patterns are of great importance.

A preferred embodiment of the present invention involves contacting the target cell with a set of 2 compounds designated as “C(1)” and “C(2)” wherein C(1) binds to the target receptor or set of target receptors designated as “R(1)” and C(2) binds to the target receptor or set of target receptors designated as “R(2)” and wherein upon the unmasking of a ligand or of a receptor, C(1) and C(2) are able to bind to together and form cross-links of the receptors R(1) and R(2).

In a preferred embodiment multiple molecules of C(1) and C(2) are able to form an aggregate on the target cell surface either directly or indirectly through the intermediacy of a third component. Only cells that have both types of receptors R(1) and R(2) can form the cross links and multi-valent aggregates that can bind to the cells with very high affinity. The very large increase in binding affinity afforded by the multi-valent binding can enable binding to cells that express both receptor types at concentrations thousands of times lower than those needed to bind to cells that express only one of the targeting receptor types. In addition the time to dissociation of multiply bound drug can be enormously increased. The mechanism of action is shown below:

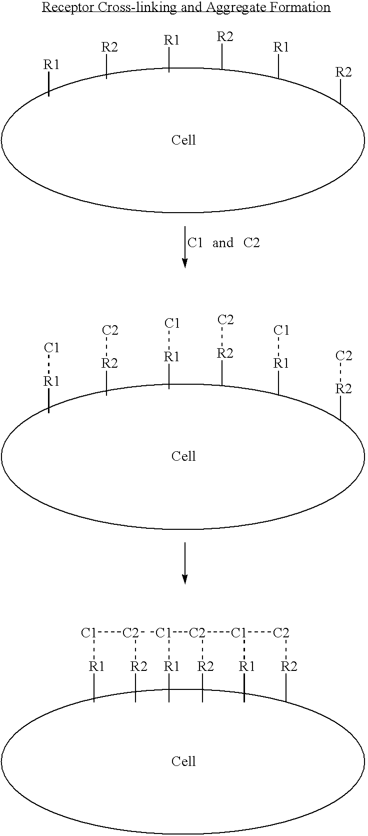

C1 and C2 can also be comprised of groups that bind to each other without the requirement that the groups be administered in a masked form. The effective concentration of membrane bound C1 and C2 can be orders of magnitude greater than the solution phase concentrations. This can enable binding to occur at the targeted cell membrane between C1 and C2 but not in the solution phase, provided that the concentration in solution is sufficiently low.

In a preferred embodiment C1 is a Compound 2 comprised of the following groups:
{[M(b)]ₘ and [E]ₒ₋₁ and [PF(a)]ₙ and T1} or
{[M(b)]ₘ and [E]ₒ₋₁ and [F(a)]ₙ and T1}
and C2 is a Compound 2 comprised of the following groups:
{[M(a)]ₘ and [E]ₒ₋₁ and [pF(b)]ₙ+T2} or
{[M(a)]ₘ and [E]ₒ₋₁ and [F(b)]ₙ+T2}
wherein T1 is a targeting agent that can bind to the receptor R1 on the target and wherein T2 is a targeting agent that can bind to the receptor R2 on the target.

The mechanism of action is illustrated below for the case in which m=2; o=2; and; n=2;

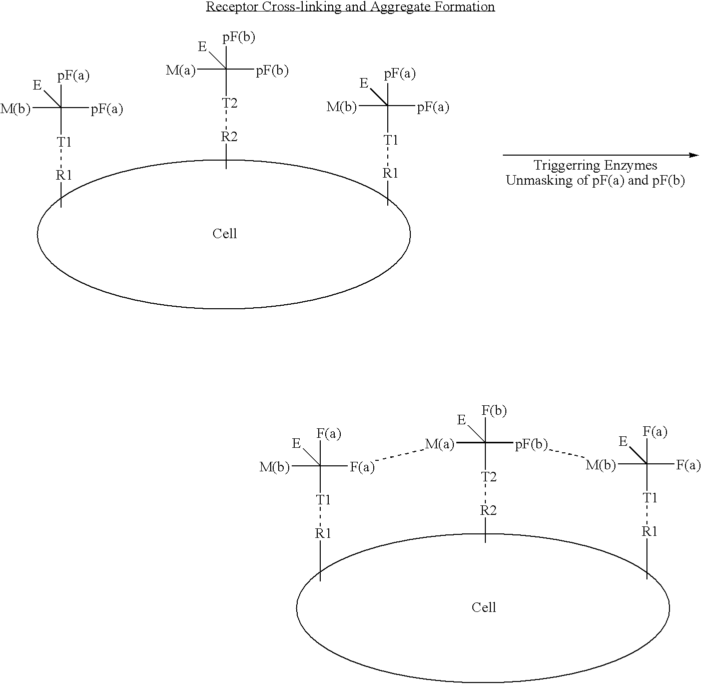

Further amplification may be achieved by the previously described mechanisms.

It should be noted that C1 and C2 are embodiments of Compound 2 in which one of the effector groups E in Compound 2 is the group T1 and T2 respectively.

The scope of the present invention includes the methods of use of the compounds described in this document and compositions of matter of the compounds individually and as compositions of matter in combination or in a kit.

One skilled in the arts will readily recognize that the present invention is broadly applicable to a wide range of compositions of Compounds 1 Compound 2 and

Compound 3. These are to be considered within the scope of the present invention. Detailed descriptions of some preferred embodiments of the groups T, E, pF, F, and M along with preferred linkers and triggers are described below:

Targeting Agents

A targeting agent “T” is comprised of a “targeting ligand” which is a chemical structure, that binds with a degree of specificity to a targeting receptor that is enriched at a target cell compared to at a non-target cell. Preferred properties for the targeting agent T in the above embodiments are as follows:
1.) The group T can bind specifically and with high affinity and to the target cell or to biomolecules in the microenvironment of the target cell.
2.) The group T should have a site for linker attachment.

T can be connected to the masked female adaptor pF(x) either directly or indirectly by a linker. The requirement for this connection is that both T and F(x) must be able to bind concurrently to their respective binding partners.

Preferred targeting agents include: monoclonal antibodies; antigen binding fragments of monoclonal antibodies; antibodies or derivatives or analogs thereof; receptor binding proteins or analogs, targeting ligands that bind to target receptors, or a chemical group that can able to bind to the target or target cell. The targeting agent may be mono-valent or multi-valent. A large number of chemical structures that can serve as targeting agents are well known to one skilled in the arts and can function in the present invention. The targeted cell receptors can be a chemical moiety that is enriched on the target cells relative to the cell populations that one desires not to target. With the advent of combinatorial chemistry, and high throughput automated screening it is now possible to select high affinity ligands that can bind to essentially any biological receptor. The following reference relates to this subject matter: Wilson, Stephen R.; Czarnik, Anthony W.(eds.), “Combinatorial Chemistry; Synthesis and Application.” John Wiley & Sons, Inc., the contents of which is incorporated herein by reference in its entirety.

The steps in this process are well known to one skilled. in the arts and include:
1.) Coupling a large library of potential receptor binding ligands to a linker and reporter functionality such as a fluorescent group, an enzyme, or a group such as biotin which can be readily detected;
2.) Coupling the receptor moiety to a solid phase;
3.) Incubating the receptor ligand-detector molecules with the receptor;
4.) Washing to remove unbound ligand; and
5.) Assaying for the reporter functionality bound to the receptor to identify high affinity binding ligands.

For example, one can couple a fluorescent derivative via a linker to a library of millions of compounds and screen potential ligands for binding affinity to the desired receptor using a fluorescent based binding assay.

Methods of ligand identification based on phage display technology are also well known to one skilled in the arts. The following reference relates to this subject matter: Walter G; Konthur Z; Lehrach H. “High-throughput screening of surface displayed gene products,” Comb Chem High Throughput Screen 2001 April; 4(2):193-205; Wright, RM, et al. “A high-capacity alkaline phosphatase reporter system for the rapid analysis of specificity and relative affinity of peptides from phage-display libraries,” J Immunol Methods Jul. 1, 2001 ;253(1-2): 223-32., the contents of which is incorporated herein by reference in its entirety.

In a preferred embodiment the targeting agent is also comprised of a second group that can also serve to localize the drug to the cell membrane. For example, a simple fatty acid group can partition into the cell membrane in a nonspecific fashion. This can contribute significantly to the binding energy of the drug to the cell and markedly increase overall target cell affinity.

The degree of amplification that can be achieved is a function of the time that the complex resides on the target. Some target receptors are known to undergo rapid internalization by endocytosis. This process although highly desirable to transport the targeted drugs into cells can if too rapid restrict the magnitude of the amplification. There are a variety of methods available to prolong the lifetime of the drug complex at the cell surface. In a preferred embodiment the targeting agent is comprised of two targeting ligahds: one that binds to a receptor that can undergo rapid endocytosis; and a second targeting ligands that binds to a target receptor that is anchored to the cell cytoskeleton. or to the extracellular matrix. The targeting agent can cross link the two receptor types and thereby anchor the drug complex and delay drug uptake. The second targeting receptor can be target cell specific or nonspecific. For example, sodium potassium ATPase is a membrane protein that is fixed to the cell cytoskeleton and has a half life for internalization of approximately 6 hours. A wide range of ligands such as oubain, digoxin, and convallotoxin, can bind to this enzyme. In a preferred embodiment T is comprised of a targeting ligand that is selective for the target cell and a second ligand that binds to sodium/potassium ATPase. In a preferred embodiment the second ligand is comprised of an inhibitor to sodium/potassium ATPase. In a preferred embodiment the ligand is comprised of a cardiac glycoside, digoxin, oubain, or convallotoxin, or digitoxin. In a preferred embodiment the site of linker attachment is to the sugar moiety. It is known that groups may be attached to the sugar moiety without impairing binding ability to the ATPase.

The method of increasing the cell surface lifetime of a complex by tethering the complex to a cell membrane component that is anchored to the cells cytoskeleton or to the extracellular matrix or which has a prolonged half-life by other mechanisms is general and is within the scope of the present invention. Other preferred receptors that can be employed for this purpose include: CD44, amelioride-sensitive Sodium channel, E-cadherin, inositol 1,4,5, triphosphate receptor, guanosine 3,5,cyclic monophosphate gated channel, and ankyrin binding membrane proteins. MMP-9 is an example of a target selective receptor that should prolong the cell surface retention of a drug complex. MMP-9 is enriched on the surface of a wide range of tumor cells and binds with high affinity to the CD44 receptor which is anchored to the cells cytoskeleton. Accordingly, a MMP-9 binding ligand should slow the rate of endocytosis of an otherwise rapidly internalized receptor complex.

In preferred embodiments of the above T is comprised of a single ligand that can bind to a receptor that is enriched on the surface of a tumor cell. In a preferred embodiment T is comprised of two targeting ligands that bind with high affinity to a pattern of targeting receptors that are enriched on target cells compared to a non target cell.

In a preferred embodiment the target is a tumor and the targeting agents are comprised of targeting ligands that bind to target receptors R; wherein either R, or the triggering enzyme, or both, are enriched at the target compared to at a non-target.

Numerous suitable ligands are described elsewhere in this document and known by one skilled in the arts. In a preferred embodiment T is comprised of two targeting ligands that are enriched on the surface of a tumor cell wherein at least one of the targeting ligands binds to a target receptor on the surface of the tumor cell or in the microenvironment of the tumor cell and wherein the tumor has an increased amount of that target receptor compared to a non-tumor cell that binds to a second targeting ligand of the compound. Generally, the increased amount is greater than about two times or greater than about 5 times, or greater than about 10 times. A preferred embodiment is comprised of targeting ligands in which at least one of the targeting ligands binds to a receptor that is absent or essentially absent from a non-tumor cell. In a preferred embodiment the pattern consisting of the receptor to which the targeting agent binds and the triggering enzyme(s) is selective to a tumor. In an even more preferred embodiment said pattern is unique to a tumor and not present in normal tissues. In another preferred embodiment the pattern is specific for both the tumor and tissue of tumor origin.

A wide range of targeting receptors that are overexpressed at tumor cells are known to one skilled in the arts. Preferred targeting ligands can bind selectively to targeting receptors that include: a cathepsin type protease; a collagenase; a gelatinase; a matrix metalloproteinase; a membrane type matrix metalloproteinase; activated Factor X; alpha v beta 3 integrin; amino-peptidase N; basic fibroblast growth factors receptors; carboxypeptidase M; cathepsin B; cathepsin D; cathepsin K; cathepsin L; cathepsin O; CD44; c-Met; CXCR4 receptor; dipeptidyl peptidase IV; emmprin; Endothelin receptor A; epidermal growth factor receptors and related proteins; epidermal growth factors; Fas ligand; fibroblast activation protein; folate receptors; gastrin/cholecystokinin type B receptor; Gastrin releasing peptide receptor; glutamate carboxypeptidase II or Prostate-specific membrane antigen; gonadotropin releasing hormone receptor; GPIlb/IIIa fibrinogen receptor; Growth hormone receptor; guanidinobenzoatase; Guanylyl cyclase C; heparanase; hepsin; human glandular kallikrein 2; insulin-like growth factor receptors; insulin-like growth factors; interleukin 6 receptor; an interleukin receptor; laminin receptor; leutinizing hormone releasing receptor; Lewis y antigen; matrilysin; matripase; melanocyte stimulating hormone receptor; multi-drug resistance protein; nerve growth factors and their receptors; neuropeptide Y receptors; neutral endopeptidase; nitrobenzylthioinosine-binding receptors (nucleoside transporter); norepenephrine transporters; nucleoside transporter proteins; opioid receptors; oxytocin receptor; patelet derived growth factor receptor; pepsin c; peripheral benzodiazepam binding receptors; p-glycoprotein; plasmin; platelet-derived growth factors and their receptors; polyamine transporters; porphyrin receptors; prolactin receptor; prostase; prostate stem cell antigen; seprase; sex hormone globulin binding receptor; sigma receptors; somatostatin receptors; SP220K; Steap antigen; stromelysin 3; sucrase-isomaltase; TADG14; thrombin; thrombin receptor; tissue factor; tissue plasminogen activator; TMPRSS2; transferrin receptors; transforming growth factors and their receptors; transporter (PEPT1); Trk receptors; trypsin; tumor necrosis factor receptor; type IV collagenase; uridine/cytidine kinase; urokinase vacuolar type proton pump (V-ATPase); a tumor-selective antigen; and a tissue specific antigen. It should be noted that targets need not be on tumor cell but can be in the microenvironment of tumor cells.

Tumor-selective Targets and Targeting Ligands:

The targeting ligands described below are some preferred embodiments of targeting ligands for anti-cancer drugs of the present invention: References that relate to the targeting ligands are provided in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs the contents of which are incorporated herein by reference in their entirety.

Laminin Receptors

The laminin receptor is a membrane associated protein which binds laminin, elastin and, type IV collagen. The receptor facilitates the cell adhesion and migration, key components of invasiveness characteristic of malignancy. The laminin receptor is over-expressed in a large number of malignancies including: breast, colon, prostate, ovarian, renal, pancreatic, melanoma, thyroid, lung, lymphomas, leukemias, gastric, and hepatocellular cancer. It is strongly associated with metastatic ability and is an independent adverse prognostic in breast, prostate, lung, thyroid and gastric cancer. In preferred embodiments the targeting ligand T comprises the following structures:

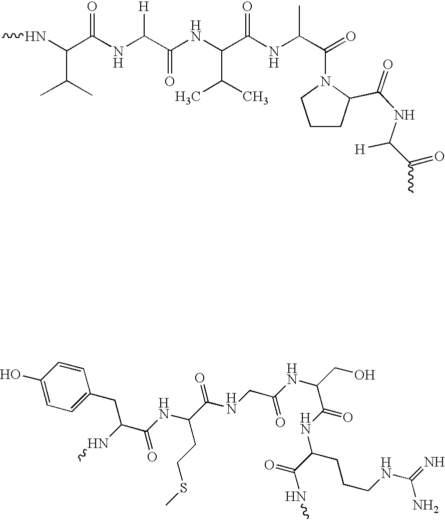

wherein the wavy line is H, OH, NH₂, or the site of linker attachment to the remainder of the drug complex; and wherein the amino acid residues have the L-configuration, or the D configuration, or are a racemic mixture.

Integrin alpha V beta 3

Integrin alpha V beta 3 (αᵥβ₃) are cell adhesion molecules which bind to RGB peptide sequences present in many extracellular matrix proteins. αᵥβ₃ is over-expressed on tumor cells in a number of important malignancies including: melanoma, breast cancer metastatic to bone, ovarian cancer, and neuroblastoma. In addition, αᵥβ₃ over-expressed by endothelial cells in tumor neovasculature. A preferred embodiment of the present invention is a Compound 1 with a targeting ligand comprised of a structure that binds to αᵥβ₃.

In preferred embodiments, T is comprised of one of the following structures: 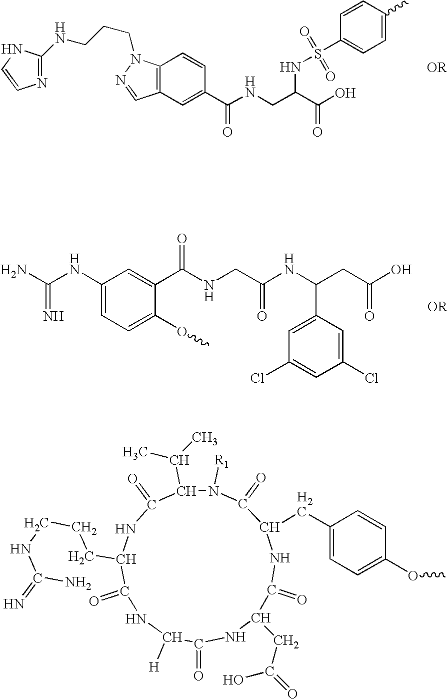
wherein the wavy line is the site of linker attachment to the remainder of the drug complex and R₁ is H, or methyl, and amino acids in the cyclopeptide are the L-configuration except for the tyrosine which is the D-configuration.
Matrix Metalloproteinases as Targets

Matrix metalloproteases (MMP) are enzymes, which degrade connective tissue and which are over-expressed by a large number of tumors and stroma of tumors. Membrane type metalloproteinases are associated with the cell surface by hydrophobic transmembrane domains or glycosylphosphatidylinositol anchors. Other MMP's become associated with the surface of tumor cells by a variety of mechanisms. In a preferred embodiment T is comprised of an MMP selective ligand.

Matrix Metalloproteinase 7 Selective Ligands:

MMP-7 is over-expressed by tumor cells in wide range of malignancies including: ovarian, gastric, prostate, colorectal, endometrial, gliomas, and breast cancer. MMP-7 contrasts with many other metalloproteases, which are over-expressed by tumor stromal elements rather than the tumor cells. In a preferred embodiment, T is a ligand for MMP-7. In preferred embodiments T is comprised of the following structures:
wherein the dotted line is the site of attachment or linker attachment to the remainder of the drug complex and wherein R1 is hydroxy, methyl, ethyl, 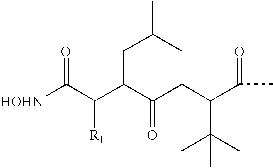
isopropyl, cyclopentyl, 3-(tetrahydrothiophenyl), or thiopen-2-ylthiomethyl.
MMP1, 2, 3, 9 and Membrane Type 1 MMP. Targeting Ligands:
MMP 1, 2, 3, 9 and membrane type MMP 1(MT-MMP-1) are all over-expressed in a wide variety of malignancies. Similarities in the active site of these enzymes allow for targeting with a common family of ligands. A preferred embodiment of the present invention is a Compound 1 with a targeting ligand comprised of a structure that binds to MMP1, 2, 3, 9 or MT-MMP-1. In preferred embodiments, T comprises the following structure: 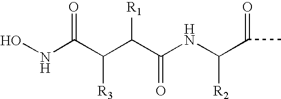
wherein the dotted line is the site of linker attachment to the remainder of the drug complex wherein R₁ is —CH₂CH(CH₃)₂, —(CH₂)₂C₆H₅, —(CH₂)₃C₆H₅, n-butyl, n-hexyl, n-octyl, R₂ is C₆H₅, ₋₋₋₋ C₆H₁₁, —C(CH₃)₃, (indol-3-yl)methyl, —CH₂C₆H₅, (5, 6, 7, 8-terahydro-1-napthyl)methyl, —CH(CH₃)₂, 1-(napthyl)methyl, 3-(napthyl)methyl, 1-(quinolyl)methyl, 3-(quinolyl)methyl, 3-pyridylmethyl, 4-pyridylmethyl, t-butyl, and R₃ is H, OH, methyl, 2-thienylthiomethyl, or allyl.

In preferred embodiments the T comprises the following structures: 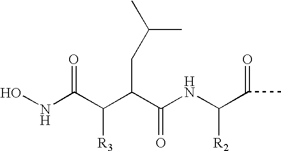
wherein R₂ is benzyl and R₃ is 2-thienylthiomethyl; or wherein R₂ is 5, 6, 7, 8,-terahydro-1-napthyl)methyl and R₃ is methyl; or wherein R₂ is t-butyl and R₃ is OH; or wherein R₂ is H and R₃ is (indol-3-yl)methyl; and wherein the dotted line is the site of linker attachment to the remainder of the drug complex.

Another preferred embodiment is based on diphenlyether sulfone inhibitors of MMP's, which are highly active against MMP2, 3, 9, 12, and 13 MMP. The following references relate to this subject matter: U.S. Pat. No. 5,932,595, Aug. 03, 1999, Bender et al., “Matrix Metalloprotease Inhibitors”; Lovejoy B., et al., “Crystal Structures of MMP-1 and -13 Reveal the Structural Basis for Selectivity of Collagenase Inhibitors,” Nat Struct Biol, 6(3):217-21 (1999); Botos I., et al., “Structure of Recombinant Mouse Collagenase-3 (MMP-13),” J Mol Biol, 292:837-844 (1999), the contents of which are incorporated herein by reference in their entirety. MMP 13 is an attractive target as it is over-expressed in a wide range of malignancies.

A preferred embodiment of the present invention is a Compound 1 with a targeting ligand comprised of a structure that binds to MMP13. In preferred embodiments T comprises the following structure: 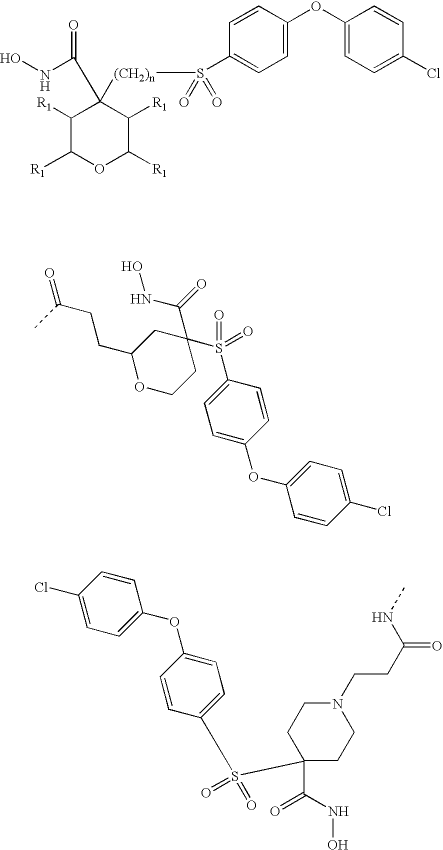
wherein n=0 or 1 and wherein R₁ is H, or the site of linker attachment to the remainder of the drug complex, and the dotted line is the site of linker attachment.
Urokinase Selective Ligands:

Urokinase is a serine protease, which converts plasminogen into enzymatically active plasmin. The enzyme binds to specific cell surface receptors and is over-expressed in most major types of cancers. A preferred embodiment of the present invention is a compound FT with a targeting ligand comprised of a structure that binds to urokinase. In preferred embodiments the targeting ligand comprises the following structure: 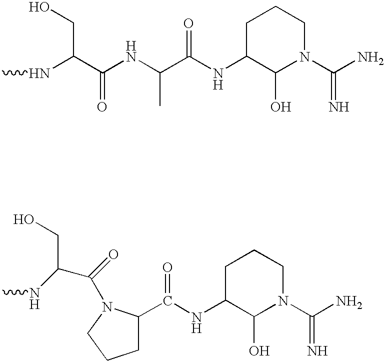
wherein the wavy line is the site of linker attachment to the remainder of the drug complex, and the serine residue has the D-configuration and the remainder of the amino acid residues has the L-configuration; or wherein the structures are L, D, or a racemic mixture.
Prostate Specific Membrane Antigen Targeting Ligands:

Prostatic adenocarcinoma cells have high concentrations of the enzyme Glutamate Carboxypeptidase II or Prostatic Specific Membrane Antigen (PSMA) on the cell surface. In addition, the enzyme is present on the brush border of the kidneys, the luminal surface of parts of the proximal small intestine and in the brain. Radiolabelled monoclonal antibodies against PSMA (ProstaScint™) are in clinical use to assess metasta tic tumor spread. PSMA has also been detected on the surface of tumor neovasculature. PSMA is a zinc carboxypeptidase, which catalyzes the hydrolysis of N-acetyl-aspartylglutamate and gamma glutamates. The enzyme is potently inhibited by phosphorous based transition state analogs. 2-(phosphonomethyl)-pentanedioic acid inhibits the enzyme with a Ki of 0.3 nanomolar. A preferred embodiment of the present invention is a compound with a targeting ligand comprised of a structure that binds to PSMA. In a preferred embodment, the targeting ligand comprises the following structure: 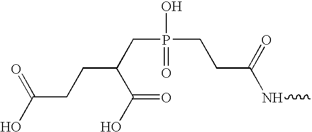
wherein the wavy line is the site of linker attachment to the remainder of the drug complex. Other preferred embodiments are based on urea based inhibitors of PSMA described by Kozikowski, A. Nan F., et al; “Design of Remarkably Simple, Yet Potent Urea-Based Inhibitors of Glutamate Carboxypeptidase II (NAALADase)”, J. of Med.Chem.; 2001; 44(3); 298-301), the contents of which are incorporated herein by reference in their entirety.

The following compound was synthesized was found to be a potent inhibitor of PSMA with an IC50=8 nM. The corresponding compound without an attached linker has an IC50=47 nM.

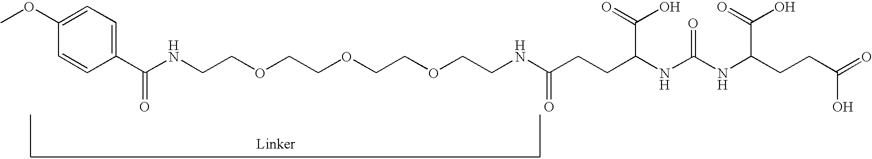

This unexpected finding demonstrates that linker attachment at the indicated site does not impair binding to PSMA and can improve affinity.

Some preferred embodiments of PSMA targeting ligands are shown below:

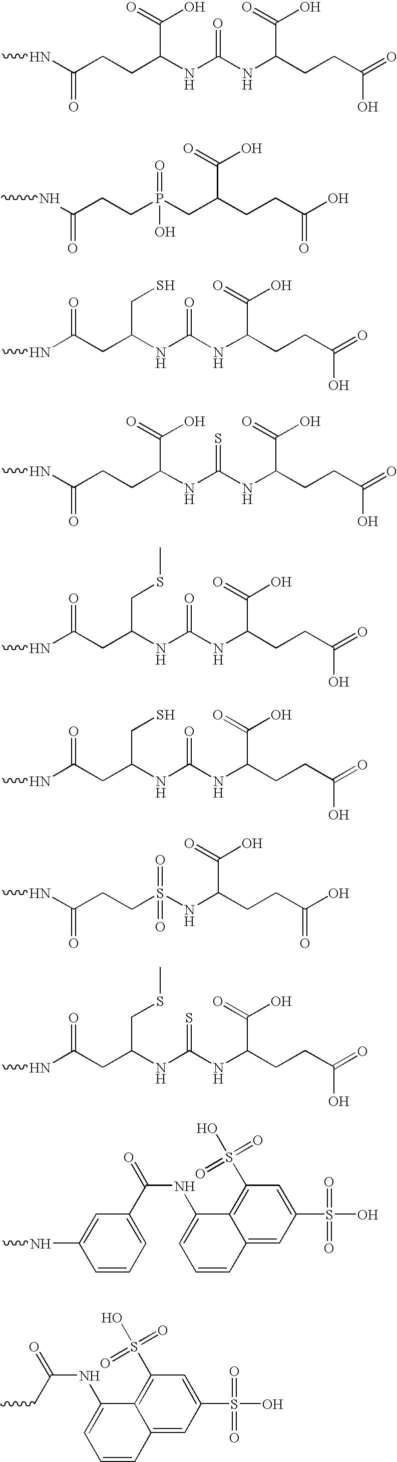

These are to be considered within the scope of the present invention. Also the present invention includes a targeted compound comprised of the above structures attached to an effector group. The method of targeting effector agents to PSMA by contacting the PSMA with a compound comprised of a targeting ligand of the above structure linked to the effector agent, is also within the scope of the present invention.

Sigma Receptor Targeting Ligands

Sigma receptors are a class of membrane-associated receptors, that are present in increased amounts on a variety of malignant tumors including: prostatic adenocarcinoma, neuroblastoma, melanoma, breast carcinoma, pheochromocytoma, renal carcinoma, colon carcinoma, and lung carcinoma. A preferred embodiment of the present invention is a Compound 1 with a targeting ligand comprised of a structure that binds to sigma receptors.

In preferred embodiments T has the following structures: 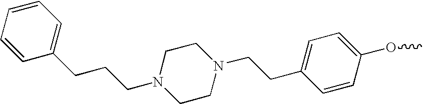 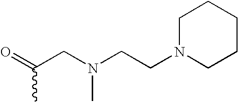
wherein the wavy line is the site of linker attachment to the remainder of the drug complex.
Somatostatin Receptor Targeted Ligands

Somatostatin receptors (SSR) are expressed at high levels in a variety of human malignancies including: breast, prostate, neuroblastoma, medullabalstoma, pancreatic, ovarian, gastrinoma, thyroid, melanoma, renal, lymphoma, glioma, colorectal, small cell lung cancer, and most neuroendocrine tumors. A preferred embodiment of the present invention is a compound with a targeting ligand comprised of a structure that binds to somatostatin receptors. A large number of somatostatin receptor selective ligands are known including octreotide, lanreotide, and vapreotide. The terminal amino group may be coupled to a linker or bulky groups with retention of binding affinity to the somatostatin receptors. Some preferred embodiments of targeting ligands are shown below wherein the wavy line is the site of linker attachment: 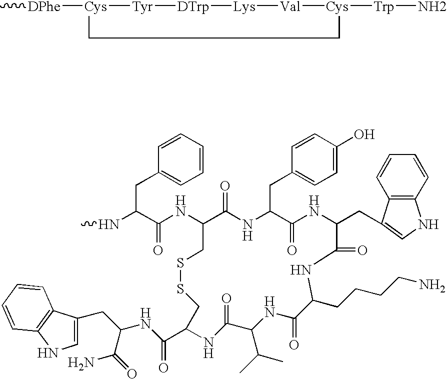
Gastrin Releasing Peptide Receptor Targeting Ligands

Gastrin releasing peptide receptors (GRPR) are over-expressed in a variety of malignancies including: lung, breast, prostate, colorectal, gastric, and melanoma. In preferred embodiments T has the following structures: 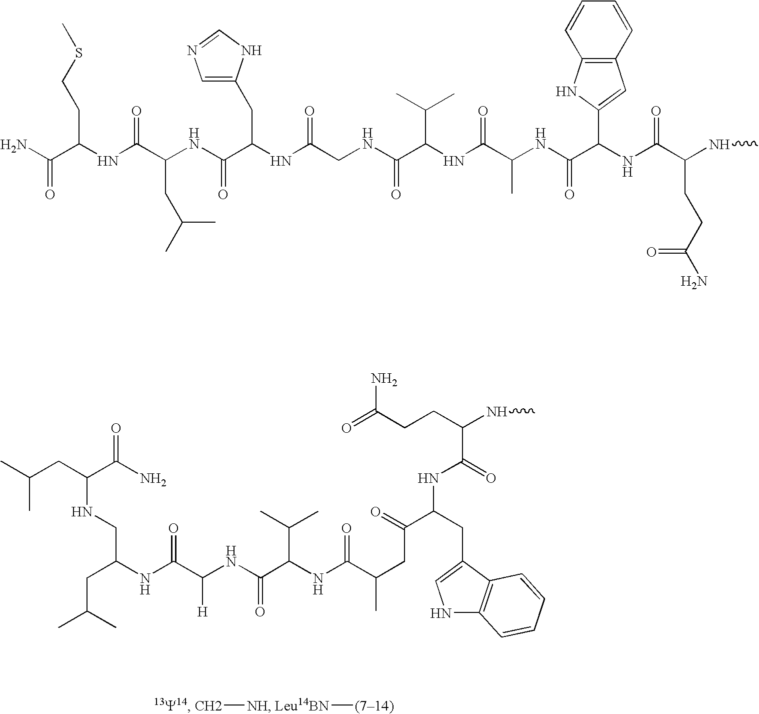
wherein the wavy line is the site of linker attachment to the remainder of the drug.
Melanocyte Stimulating Hormone Receptor Targeting Ligands

Melanocyte Stimulating Hormone Receptors (MSHR) bind melanocyte stimulating hormone and related peptide factors with high affinity. The consistent expression of MSHR in malignant melanoma has stimulated efforts to employ the receptor for diagnostic imaging and chemotherapy targeting. A preferred embodiment of the present invention is a Compound 1 with a targeting ligand comprised of a structure that binds to MSHR. Preferred embodiments of T are based on some melanotropin analogs, which possess extremely high receptor affinity. In preferred embodiments T has the following structures: 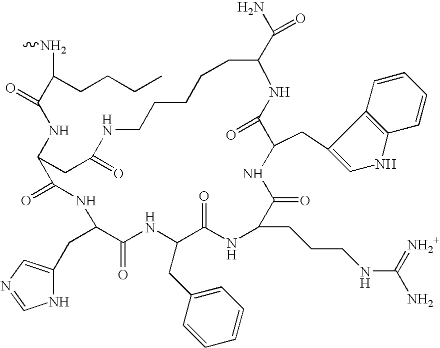
wherein the wavy line is the site of linker attachment to the remainder of the drug complex.
Luteinizing Hormone Releasing Hormone Receptors Selective Ligands

LHRH receptors are present in the majority of cases of prostate cancer. In a series of primary prostate cancer specimens 69/80 were positive for LHRH receptors. LHRH are also present in ovarian cancer, breast cancer, and endometrial cancer.

A preferred embodiment of T is:
pGlu-His-Trp-Ser-Try-D-Lys-Leu-Arg-Pro-Gly-NH₂
wherein the linker is attached to the amino group of the D-Lys residue. The following references relate to this subject matter: Nagy A., et al., “Cytotoxic Analogs of Luteinizing Hormone-Releasing Hormone Containing Doxorubicin or 2-Pyrrolinodoxorubicin, a Derivative 500-1000 Times More Potent”, Proc Natl Acad Sci USA, 93:7269-7273 (1996) the contents of which are incorporated herein by reference in their entirety.
Linkers

A large variety of chemical structures can be employed as linkers to connect different functional groups of the compounds together. Considerations for the selection of linkers designated as “L” are as follows:
1) L should have chemical groups that allow it to be covalently coupled to the components of the compound. The covalently coupling preferably should not significantly interfere with the function of the attached components;
2) For some but not all embodiments, L should be of sufficient length to allow for crosslinking of targeting receptors;
3) L can preferably be inert in the sense that L should generally not bind with high affinity to cells or tissue components;
4) L should be sufficiently chemically stable to allow the drug to reach its target site functionally intact;
5) L can also have sites to which groups that allow manipulation of drug solubility can be attached; and
6) L preferably should have low immunogenicity.

Linkers with water solubility are especially preferred. Similar requirements apply to linkers used to couple other components of the drug molecule together. The optimal length of the linkers can vary depending on the structure of the receptors. The expected range is from one up to about 350 bond lengths or from 1 to about 10 bond lengths, or from about 10 to about 40 bond lengths, or from about 20 to about 80 bond lengths, or from about 80 to about 150 bond lengths, or from about 150 to about 350 bond lengths, or 1, 2, 3, 4, 5, 6, 7, 8, 9, 10, 11, 12, 13, 14 . . . 350 or about 350 bond lengths; wherein the dots are used to represent the individual numbers in the sequence between 14 and 350. The linkers may also be polymers with a distribution about the average linker lengths given above. The linkers can be comprised of oligo or poly-ethylene glycols—(O—CH₂-CH₂-)n- with (n=1, 2, 3, 4, 5, 6, 7, 8, 9, 10, 11 . . . or 120 or about 120), glycols, oligo or polypropylene glycols, polypeptides, oligopeptides polynuclueotides, oligonucleotides, —(CH₂)n-, with (n=1, 2, 3, 4, 5, 6, 7, 8, 9, 10, 11 . . . or 25 or about 25). The linker can have groups that increase water solubility. Preferred embodiments of such groups comprise: phosphates, phosphonates, phosphinates, sulfonates, carboxylates, amines, hydroxy groups, and polyalcohols. Linkers with structural rigidity are also well known to one skilled in the arts and can enhance function by decreasing negative entropic effects. The linker can be connected to the other components by a large variety of chemical bonds. Preferred functionalities include, but are not limited to: carboxylate esters and amides, amides, ethers, carbon- carbon, disulfides, —S—S—S—, acetals, esters of phosphates, esters of phosphinates, esters of phosphonates, carbanates, ureas, N—C bonds, thioethers, sulfonamides, and thioureas. Especially preferred are amide bonds and carbamates.

Linkers can be linear or can be nonlinear with branches. Linkers can be dendrimers. Linkers can be comprised of shorter linkers that are covalently joined. In preferred embodiments the covalent joining is at a multivalent molecule to which multiple linkers can be coupled. Preferred embodiments are molecules that have multiple chemical functionalities such as amino, carboxylate, hydroxy, —SH, isocyanate, and isothiocyanate that can be reacted with the linker to form a covalent bond. Preferred embodiments include: L-amino acids, D-amino acids, or racemic mixtures thereof, amino acid analogs, lysine, aspartic acid, cysteine, glutamic acid, serine, homoserine, hydroxyproline, ornithine, tyrosine, Kemps acid; multiply substituted benzene rings, glycerol, pentaerithrol, erithol, and citric acid, cyclodextrin; or cyclodextrin analogs and derivatives. Oligopeptides, peptides, proteins, and olgo-inucleotides and analogs thereof, can also serve as sites to which individual linker elements are attached. One skilled in the arts would readily recognize a very large number of other polyfunctional molecules that can be employed to connect smaller linkers together.

Examples of molecules that are suitable for use as linkers or as molecules to join together multiple linkers can be found in the Aldrich Chemical Catalog (2000) of Sigma-Aldrich Co. and the Shearwater Polymers, Inc. Catalog “Functionalized Biocompatible Polymers for Research and Pharmaceuticals. Polyethylene Glycol and Derivatives,” (2000), and a large number of suitable linkers and references to linkers are detailed in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs” the contents of which are hereby incorporated by reference in their entirety.

Some preferred embodiments of linkers are shown below: 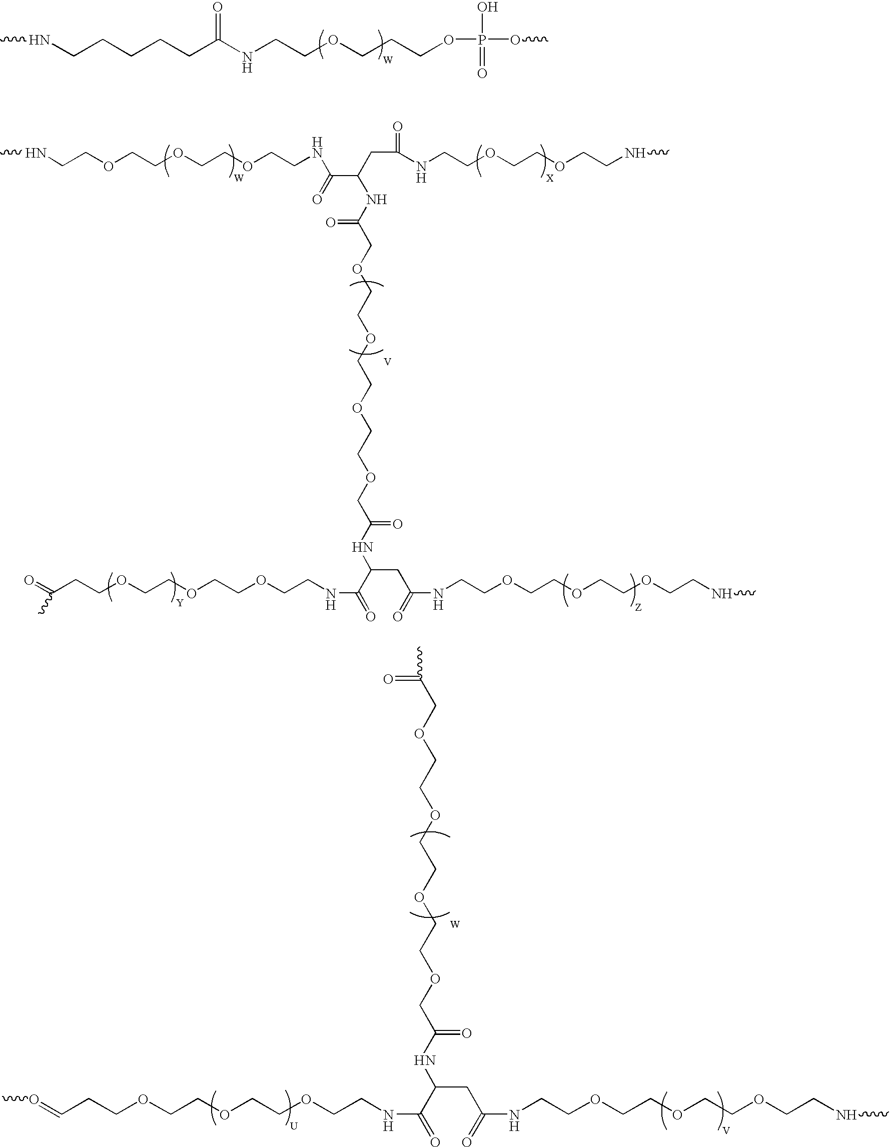 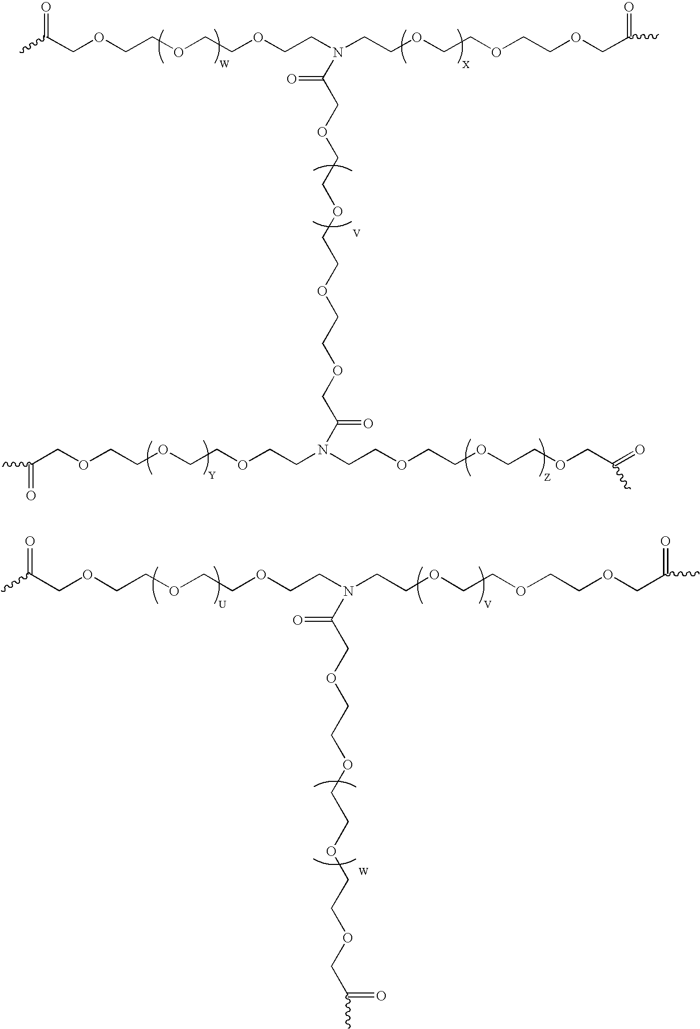 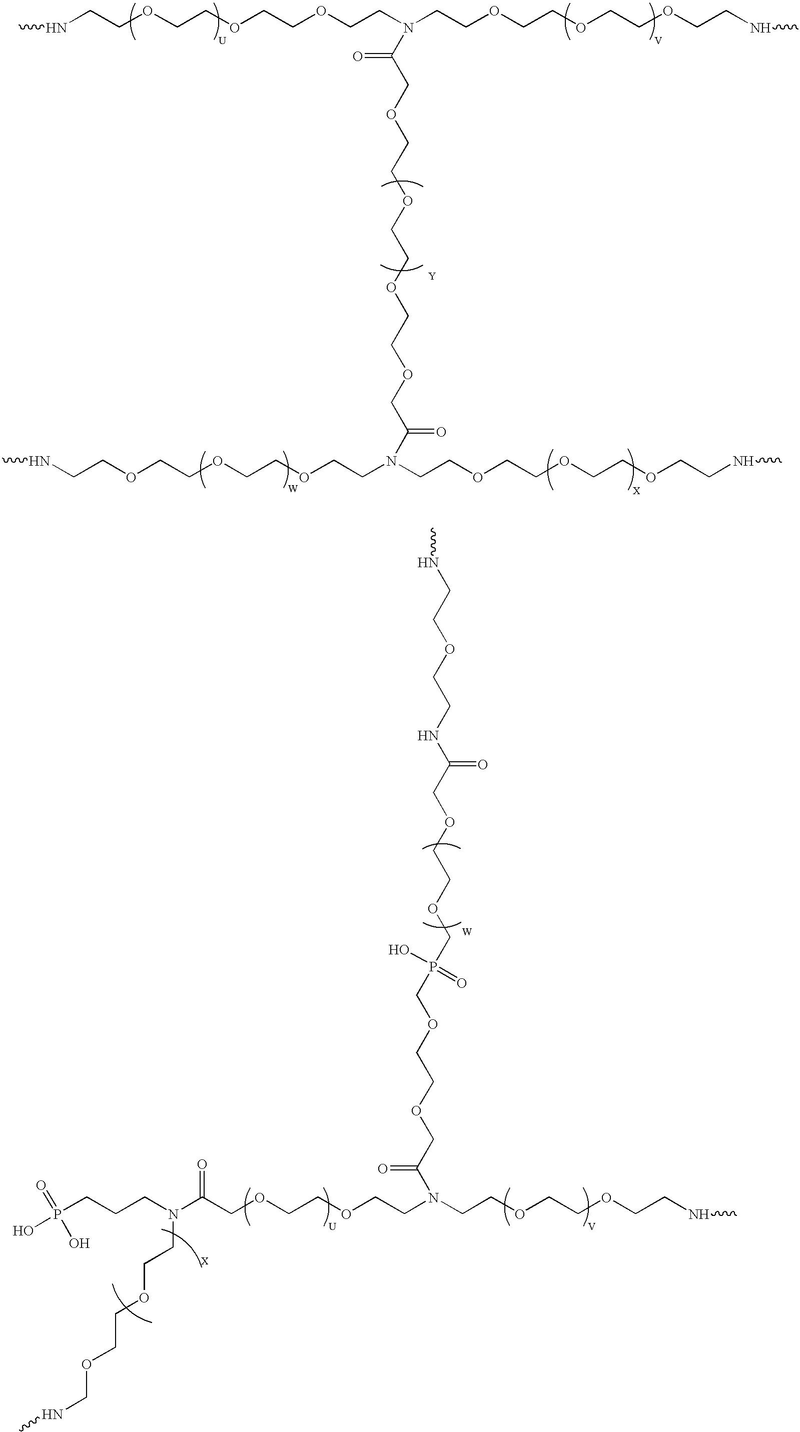 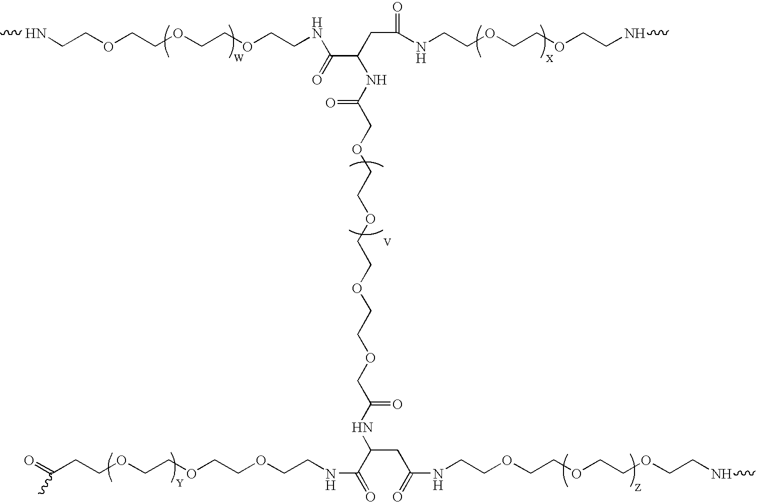
where U=0, 1, 2, 3, 4, 5, 6, . . . 150 or about 150;
where V=0, 1, 2, 3, 4, 5, 6, . . . 150 or about 150;
where w=0, 1, 2, 3, 4, 5, 6, . . . 150 or about 150;
where x=0, 1, 2, 3, 4, 5, 6, . . . 150 or about 150;
where y=0, 1, 2, 3, 4, 5, 6, . . . 150 or about 150;
where z=0, 1, 2, 3, 4, 5, 6, . . . 150 or about 150;
and wherein the wavy lines are the sites of attachment of the linkers to other components.

Additional preferred embodiments of linkers are comprised of the following structures: 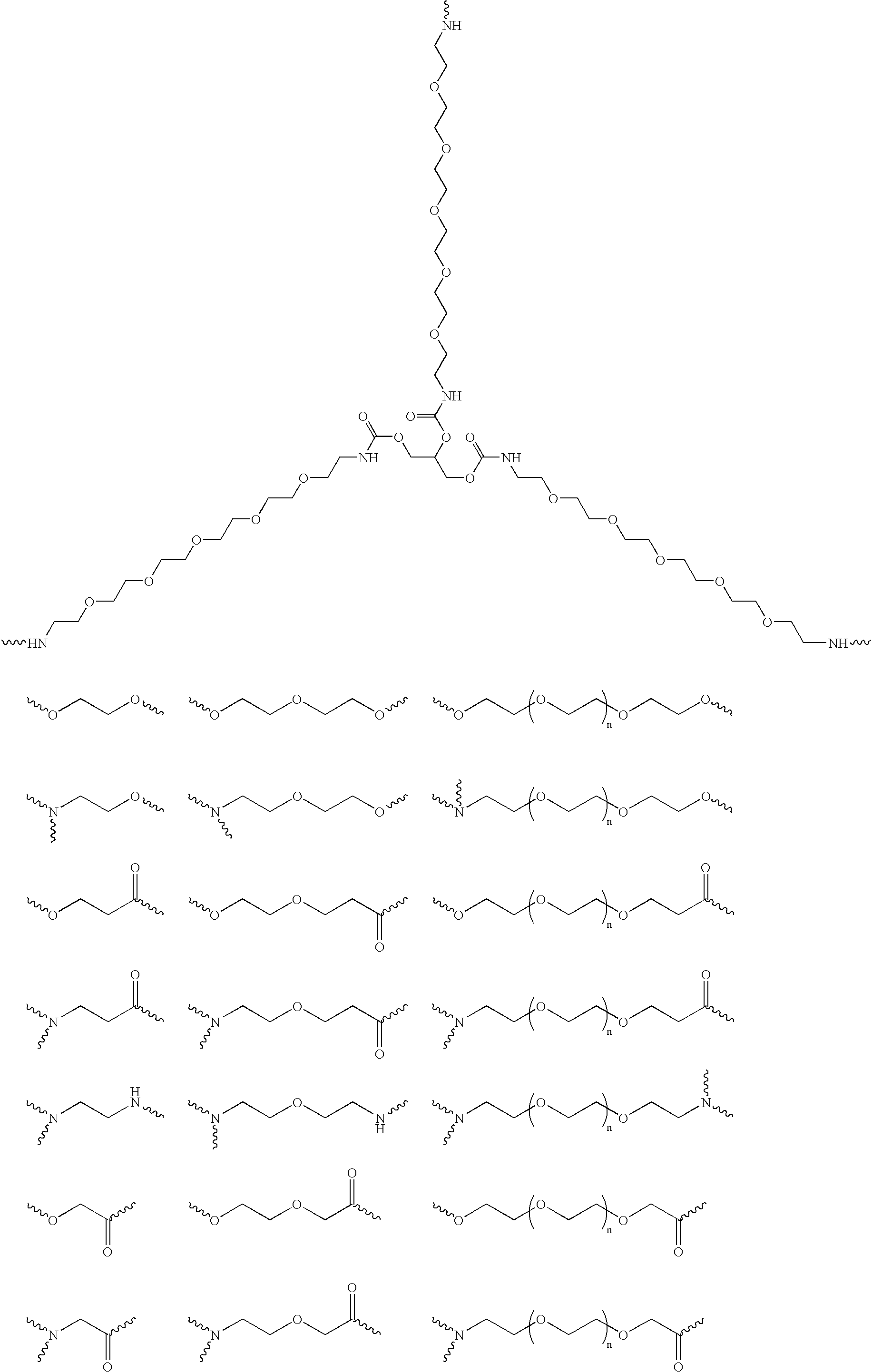 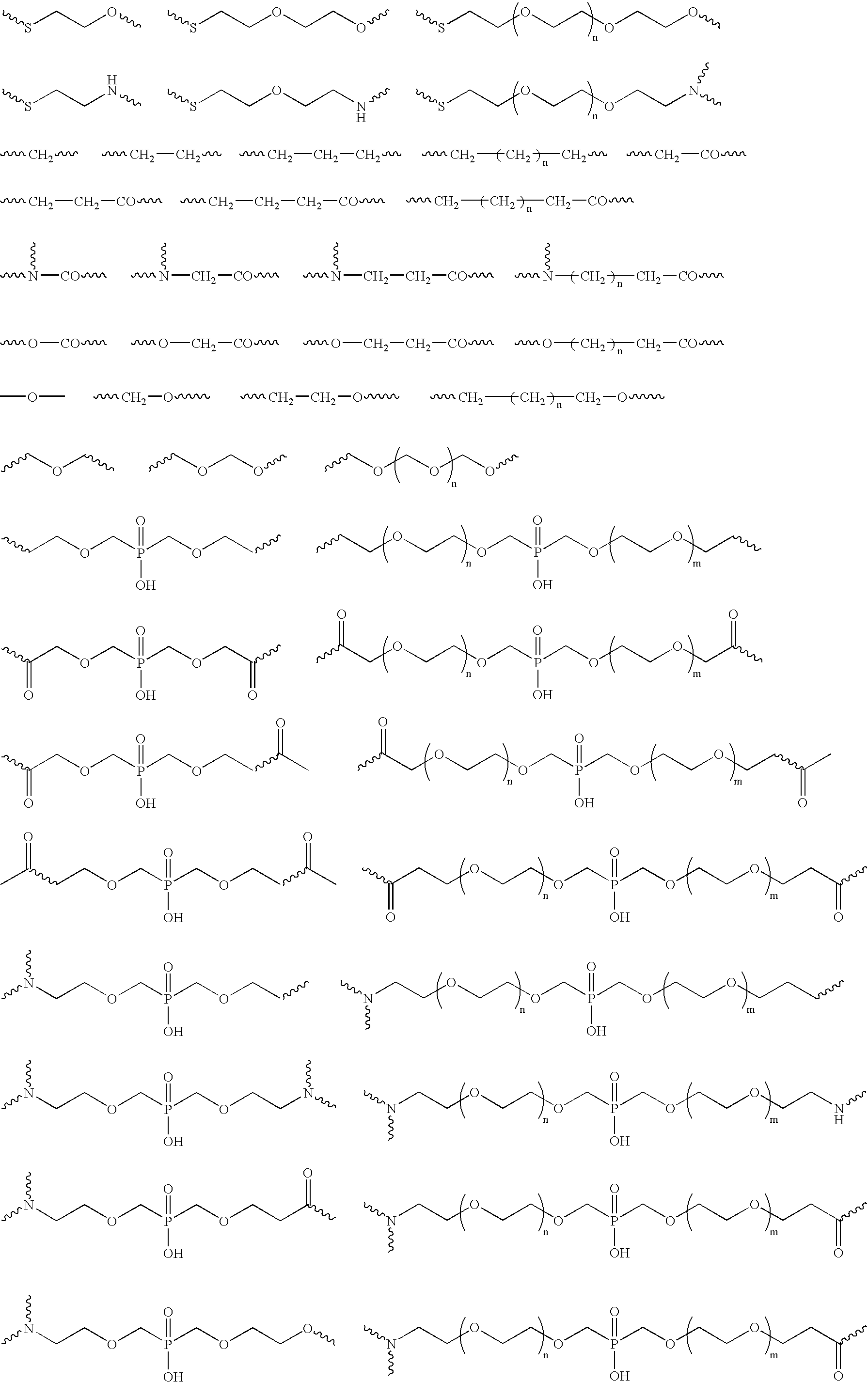 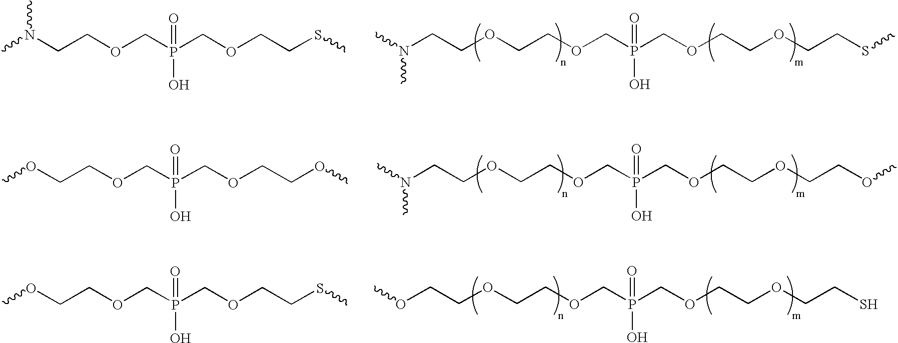
wherein the wavy line is the site of linker attachment to the components or may be H, and wherein m=0, 1, 2, 3, 4, 5, 6, . . . 150 or about 150;
and wherein n=0, 1, 2, 3, 4, 5, 6, . . . 150 or about 150;
and wherein the linkers can also be connected to each other or to multi-functional joiner molecules as described above.
Effector Mechanisms and Effector Agents Diagnostic Applications:

The present invention, can be employed to deliver an enormous range of effector agents E, depending on the intended drug indication. For diagnostic purposes, E can be comprised of a wide range of entities that allow for detection using imaging techniques commonly employed in radiology and nuclear medicine. The following reference relates to this subject matter: Reichert D. E., et al., “Metal Complexes as Diagnostic Tools,” Coordination Chemistry Reviews, 184:3-66 (1999); the contents of which is hereby incorporated by reference in its entirety.

Examples include, radioactive moieties, ligands that bind radioisotopes, groups applicable to positron emission tomography, and groups applicable to magnetic resonance imaging, such as gadolinium chelates. The detector group can also be an enzyme, a fluorescent moiety, or a group such as biotin, which is amenable to histochemical detection for the applications related to histopathology.

Therapeutic Applications

Although the principle application of this invention is in the area of anti-cancer therapy, the invention can be applied to many other areas of drug delivery. For example, the targeting methodology can be used to deliver a cytotoxic agent to a selected class of lymphocytes for the treatment of an autoimmune disease such as scleroderma or lupus erythematosis. The targeting technology can also be used to deliver a therapeutically useful drug, enzyme, protein, radionuclide, or polynucleotide or oligonucleotide or analogs thereof, or immunostimulatory molecule.

Anti-cancer Agents

A wide range of anti-cancer drugs can be selectively targeted to tumor cells with the present invention. The high target affinity of the drug for tumor cells can potentially allow a reduction in the total drug dose employed by a factor of 1000 to perhaps 1 million fold compared to non-targeted drug. At these low doses toxicity of the non-targeted drugs generated by metabolism of the targeted drug can be completely inconsequential. Toxins directed specifically against the key enzymes of cell replication are preferred. These include inhibitors to: thymidylate synthase, DNA polymerase alpha, Toposisomerase I and II, ribonucleotide reductase, Thymidylate kinase, cyclin dependent kinases, DNA primase, DNA helicase, and microtubule function.

Preferred toxins include: anthracyclines, ellipticines, taxols, mitoxantrones, epothilones, quinazoline inhibitors of thymidylate synthase, stautosporin, podophyllotoxins, bleomycin, aphidicolin, cryptophycin-52, mitomycin c, phosphoramide mustard analogs, vincristine, vinblastine, indanocine, methotrexate, 2-pyrrolinodoxorubicin, Doxorubicin mono-oxazolidine, Chromomycin A3, Wortmannin; Maytansinoids; Dolastatin 10 anologs, α Amanitin, (5-Amino-1H-indol-2-yl)-(1-chloromethyl-5-hydroxy- 1,2-dihydro-benzo[e]indol-3-yl)-methanone and analogs thereof; radionuclides, valinomycin, ionophores, convallotoxin, oubain, saponins, digoxin, filipin, thapsigargin analogs, and compounds with cytotoxicity for cells in the 10 micromolar range or lower that are currently listed in the U.S. National Cancer Institute's Developmental Therapeutics Program's, Human Tumor Cell Line Screen for Anti-cancer Agents data base which is accessible at http://dtp.nci.nih.gov/ and is hereby incorporated in its entirety by reference. The amplification that results from the present invention can enable drugs of very low cytotoxicity to kill tumor cells. Most current anticancer drugs are highly toxic, mutagenic, carcinogenic, and teratogenic. The occurrence of second malignancies induced by chemotherapy is a significant clinical problem. The present invention should enable the destruction of tumor cells with agents of low toxicity that do not cause DNA damage and therefore should not increase the risk of second malignancies. The ability to employ agents that do not damage DNA should be especially useful in men and women who desire to have children. The ability to treat cancer with targeted drugs of low toxicity that do not cause genetic damage can also shift the risk benefit ratio and allow patients who are at low risk of tumor recurrence to receive therapy.

In a preferred embodiment the effector groups are membrane active compounds that disrupt membrane integrity. Agents that are able to induce cell lysis by damaging the structural integrity of membranes are well known to one skilled in the arts and include agents such as saponin, filipin, ionophores, polyene antibiotics, valinomycin, lytic peptides, alamethicin, free radical generators.

The scope of the present invention also includes the case where E is comprised of a protein, an enzyme, oligopeptide analog, oligonucleotide analog, polynucleotide analog, viral vector, or other molecular species, which would benefit from the targeted delivery methods. The generality of the method can allow most types of diagnostic or therapeutic molecules to be employed as effector agents E.

In a preferred embodiment E is comprised of a group, with a therapeutic radioisotope or a boron-bearing group, for use in neutron capture therapy. The group E can be a wide range of radionuclide bearing groups or chelates examples of which are well known to one skilled in the arts. The following reference relates to this matter: Mattes MJ.; “Radionuclide-antibody conjugates for single-cell cytotoxicity.” Cancer (2002) 94(4 Suppl):1215-23; and McDevitt M R, Ma D, Lai L T, Simon J, Borchardt P, Frank R K, Wu K, Pellegrini V, Curcio M J, Miederer M, Bander N H, Scheinberg D A; “Tumor therapy with targeted atomic nanogenerators”; Science Nov. 16, 2001 ;294(5546):1537-40; the contents of which are incorporated herein by reference in their entirety.

The effector agent E can also be comprised of a ligand that binds to an enzyme or receptor. For example by incorporating a group E that can bind to the triggering enzyme that unmasks the group pF the effective concentration of the enzyme and therefore the rate of trigger activation can be enormously increased. For example, simple amino bearing groups such as lysine bind plasmin with high affinity. In a preferred embodiment a group E that is comprised of a lysine and preferably a lysine at the carboxy terminus of an oligo-peptide or analog thereof. Many ligands that bind potential triggering enzymes are well known to one skilled in the arts or can be identified by routine methods of ligand identification previously described. These embodiments are to be considered within the scope of the present invention.

The present invention also includes a method to increase the rate of enzymatic activation of a substrate or masked female adaptor comprising coupling to said substrate or masked female adaptor a ligand that can bind the triggering enzyme and thereby increase the effective enzyme concentration at the substrate or receptor site.

E can be connected to the drug complex either by a trigger, that when activated releases it; or E can be connected in a stable fashion directly to a linker. The mode of connection depends upon the requirements for E to exert its effector function. For example, if E is a radioisotope liberation form the target drug complex is unnecessary for activity.

Preferably the connection of the effector agent to the remainder of the drug should be by chemical groups that are sufficiently stable in vivo to allow the drug to reach the target site intact. If the effector agent can evoke its intended pharmacological activity while still attached to the remainder of the molecule than it is preferable that the connection of E be by a chemical linkage that is resistant or significantly resistant to cleavage in vivo. Examples of preferred chemical linkages for this case include: C—C bonds; ether bonds; amides; carbamates; thioethers; C—N bonds; and ureas. A very large number of suitable drugs that can serve as effector agent E and methods to couple these drugs to linkers are well known to one skilled in the arts. A large number of such methods are given in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs”.

In a preferred embodiment the effector agent E is a cytotoxic drug that is connected to a trigger that is connected to a linker that is connected to the remainder of the drug. In a preferred embodiment the trigger is a group that can be preferentially modified or activated inside cells and releases the cytotoxin inside the cell. Preferred embodiments of triggers are described in the trigger section. In a preferred embodiment the connection of E can be by a chemical linkage that is resistant or significantly resistant to cleavage in vivo but which is cleaved upon in vivo modification or activation of a trigger group. Preferred chemical linkages of an effector agent to a trigger are by chemical groups such as carbamates, amides, acetals, and ketals, phosphotriesters, phosphonate diesters, and disulfides. Other functionalities such as esters, carbonates, or other type of chemical linkage that is sufficiently stable in vivo to allow the drug to reach the target site substantially intact may be employed.

In a preferred embodiment of the invention multiple different types of Compound 2 with different independent cytotoxic agents are administered concurrently. The result can be a co-aggregate on the tumor cell surface that contains a mixture of each Compound 2 with its respective cytotoxic agents. If the cytotoxic agents are selected to have independent mechanisms of cell resistance than the probability that a tumor cell can be resistant to all the drugs is the product of the probabilities which can become vanishing small. In preferred embodiments the number of different Compound 2 types employed that differ in the group E are 2, 3, 4, 5, or 6. In a preferred embodiment the effector groups are selected such that the agents exert synergistic toxicity. A large number of agents that exert synergistic toxicity are known and are described in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs”. In a preferred embodiment, the targeting ligands are selective for receptors increased on tumor cells and the effector agents are drugs that exert synergistic toxicity.

Adaptors F(x) and Ligands M(x)

A large number of receptor ligand pairs may be employed as F(x) and M(x). The key requirements are as follows:
1.) M(x) and F(x) should bind together specifically and with sufficient affinity that aggregation of Compound 1 and Compound 2 can occur at the target at concentrations of Compound 2 that are generally nontoxic and systemically achievable.
2.) Both F(x) and M(x) should have sites to which a linker may be attached that enable the groups to be coupled to the remainder of the targeted molecule and such that the affinity for each other remains intact.
3.) Preferably F(x) should have one or more sites to which a masking group can be attached such that the masking group impairs binding to M(x).

The mechanism of binding between F(x) may be noncovalent; covalent or a combination of both types of bonding. Preferably, the affinity of F(x) and M(x) are sufficiently high such that the complex has a very long half-life and is essentially irreversible. One skilled in the arts can recognize many groups that can bind specifically and with sufficient affinity to serve as F(x) and M(x). The same screening technologies described above that are well known for ligand identification can also be applied to identify pairs of compounds that can serve as the basis for the groups F(x) and M(x) or the groups f(k) and m(k) described below.

Preferred embodiments include F(x) and M(x) comprised of:
1.) Biotin and a biotin binding protein such as avidin or streptavidin and;
2.) A monoclonal antibody, or an analog thereof, or an antigen binding Fab fragment, and a hapten that binds to said compound and;
3.) An oligonucleotide or a polynucleotide, or an analog thereof comprised of purine and or pyrimidine bases; and a complementary binding oligo or polynucleotide; and
4.) A dimer or trimer of vancomycin and a dimer or trimer of the dipeptide comprised of D alanine or analogs thereof.
5.) oligonucleotide aptmers
6.) Groups and multimers of groups that are able to engage in multi-site complementary hydrogen bonding.

The following references relate to the above matter: Rao, Jianghong, et al. “A Trivalent System from Vancomycin D-Ala-D-Ala with Higher Affinity Than Avidin Biotin,” Science 280 (1 May 1998); and Famulok, Michael, Rao, Jianghong and Whitesides, George M. “Tight Binding of a Dimeric L-Lys-D-Ala-D-Ala,” J. Am. Chem. Soc. 119: 1.0286-10290 (1997 “Oligonucleotide aptamers that recognize small molecules,” Current Opinion in Structural Biology 9:324-329 (1999);and Zimmerman, Steven C., Corbin, Perry S. “Heteroaromatic Modules for Self-Assembly Using Multiple Hydrogen Bonds.“In Fujita, M., ed.,” Struct. Bond. 96, Springer-Verlag 2000; the contents of which are incorporated herein by reference in their entirety.

Small low molecular weight groups are preferred for F(x) and M(x). In a preferred embodiment the groups F(x) and M(x) are comprised of k subunits designated as “f(k)” and “m(k)” wherein k=1, or 2, or 3, or 4, or 5, or 6, or 7, or 8, or , 9, or 10, or about 10; and wherein f(k) binds to m(k); and wherein the multi-valent binding between the subunits result in very high total binding affinity between F(x) and M(x). Preferred embodiments of f(k) and m(k) include:
1.) An oligonucleotide or a polynucleotide, or an analog thereof comprised of purine and or pyrimidine bases; and a complementary binding oligo or polynucleotide; and
2.) A glycopeptide antibiotic such as vancomycin, and a glycopeptide antibiotic binding peptide such as a dipeptide comprised of D-alanine.
3.) Groups and multimers of groups that are able to engage in multi-site complementary hydrogen bonding
   Oligo-nucleotide and Poly-nucleotide based Groups

In a preferred embodiment of M(x) and F(x) and m(k) and f(k) the groups are comprised of complementary oligo or poly-nucleotides or analogs or derivatives thereof. The sequence of the bases is not important provided that the respective sequences are complementary and can bind with sufficient affinity. Oligo and poly-nucleotides can rapidly bind with high affinity high specificity by Watson-Crick base pairing or by Hoogsteen base pairing. In a preferred embodiment the linker is attached at a terminus of the oligo-or poly-nucleotide. Linker attachment at this site will not impair base recognition and binding affinity. The length of the oligo or polynucleotide and base composition are key factors in determining the binding affinity. In preferred embodiments the length in base units is X where X=3 4,5,6,7,8,9,10,11,12,13,14,15,16,17,18,19,20,21,22,23, 24,25,26,27,28,29,30,31,32,33,34,35,36,37,38,39,40, . . . 100 or about 100. In other preferred embodiments the length in base units is with a range of about 4-10, 10-20, 20-40, or 40-100. In a preferred embodiment the oligo or polynucleotide is comprises a strand which is resistant to enzymatic degradation by nucleases. A wide range of nuclease resistant oligonucleotides are well known to one skilled in the arts. Preferred compositions of the oligo and polynucleotides include:
1.) Conventional single stranded DNA or RNA
2.) Poly-amide nucleic acids (PNA) or peptide nucleotide analogs
3.) 2′-O-{2-[N,N,-(dimethyl)aminoxoyl]ethyl} modified oligonucleotides
4.) 2′-O-{2-[N,N,-(diethyl)aminoxoyl]ethyl} modified oligonucleotides.
5.) Locked nucleic acids
6.) Phosphoramidate analogs of single strand RNA or DNA
7.) Phosphorothioate analogs of single strand RNA or DNA
8.) Methylphosphonate analogs of single strand RNA or DNA
9.) 2-O-methyl single stranded RNA analogs
10.) Phosphono PNA nucleic acid analogs
11.) Formacetal DNA and RNA analogs
12.) Thioformacetal DNA and RNA anaolgs
13.) Methylhydroxylamine DNA and RNA anaolgs
14.) Oxime DNA and RNA analogs
15.) Methylenedimethylhydrazo DNA and RNA anlogs
16.) Dimethylenesulfone DNA and RNA analogs
17.) Morpholino DNA and RNA analogs
18.) Methylene methylinino DNA and RNA analogs
19.) DNA and RNA anlogs with urea linkages
20.) DNA and RNA anlogs with guanidino linkages
21.) 2′ ribose modified RNA anlogs,such as 2′-fluoro, 2-O-propyl, 2′-O-methoxyethyl, 2′-aminopropyl
22.) DNA and RNA analogs comprised of α nucleosides
23.) Nucleic acid analogs comprised of combinations of the above

The oligonucleotide analogs may be substituted with groups that enhance water solubility provided that said groups are inert and do not interfere with binding affinity. The following references relate to the above matter: Praseuth, D., et al. “Triple helix formation and the antigene strategy for sequence-specific control of gene expression,” Biochimica et Biophysica Acta 1489:181-206 (1999); Linkletter, Barry A., and Bruice, Thomas C. “Solid-phase Synthesis of Positively Charged Deoxynucleic Guanidine (DNG) Modified Oligonucleotides Containing Neutral Urea Linkages: Effect of Charge Deletions on Binding and Fidelity,” Bioorganic & Medicinal Chemistry 8:1893-1901 (2000); Morvan, François, et al. “Oligonucleotide Mimics for Antisense Therapeutics: Solution Phase and Automated Solid-Support Synthesis of MMI Linked Oligomers,” J. Am. Chem. Soc. 118:255-256 (1996); Wang, Jianying and Matteucci, Mark D., “The Synthesis and Binding Properties of Oligonucleotide Analogs Containing Diastereomerically Pure Conformationally Restricted Acetal Linkages,” Bioorganic & Medicinal Chemistry Letters 7(2):229-232 (1997); Fujii, Masayuki, et al., “Nucleic Acid Analog Peptide (NAAP) 2. Syntheses and Properties of Novel DNA Analog Peptides Containing Nucleobase Linked β-Ainoalanine,” Bioorganic & Medicinal Chemistry Letters 7(5):637-640 (1997); Dempcy, Robert O., et al., “Design and synthesis of deoxynuclieic guanidine: A polycation analogue of DNA,” Proc. Natl. Acad. Sc. USA 91:7864-7868 (August 1994); Sabahi, Ali, et al., “Hybridization of 2′-ribose modified mixed-sequence oligonucleotides: thermodynamic and kinetic studies,” Nucleic Acids Research 29(10):2163-2170 (2001); Wahlestedt, Claes, et al., “Potent and nontoxic antisense oligonucleotides containing locked nucleic acids,” Proc. Natl. Acad. Sc. USA 97(10): 5633-5638 (May 9, 2000); Efimov, Vladimir A., et al., “Synthesis and evaluation of some properties of chimeric oligomers containing PNA and phosphono-PNA residues,” Nucleic Acids Research 26(2): 566-575 (1998); Geary, Richard S., et al., “Pharmacokinetic Properties of 2′-O-(2-Methoxyethyl)-Modified Ogligonucleotide Analogs in Rats,” The Journal of Pharmacology and Experimental Therapeutics 296(3): 890-897 (2001); Nawrot, Barbara et al., “Novel internucleotide 3′-NH—P(CH₃)(O)-0-5′ linkage. Oligo(deoxyribonucleoside methanephosphonamidates); synthesis, structure and hybridization properties,” Nucleic Acids Research 26(11): 2650-2658 (1998); Larsen, H. Jakob, and Nielsen, Peter E., “Transcription-mediated binding of peptide nucleic acid (PNA) to double-stranded DNA: sequence-specific suicide transcription,” Nucleic Acids Research 24(3): 458-463 (1996); Egholm, Michael, et al., “PNA hybridizes to complementary oligonucleotides obeying the Watson-Crick hydrogen-bonding rules,” Nature 365: 566-568 (Oct. 7, 1993); Nielsen, Peter E., et al., “Sequence-Selective Recognition of DNA by Strand Displacement with a Thymine-Substituted Polyamide,” Science 254:1497-1500 (Dec. 6, 1991); Schwarz, Frederick P., et al., “Thermodynamic comparison of PNA/DNA and DNA/DNA hybridization reactions at ambient temperature,” Nucleic Acids Research 27(4): 4792-4800 (1999); Jensen, Kristine Kilså, et al., “Kinetics for Hybridization of Peptide Nucleic Acids (PNA) with DNA and RNA Studied with the BIAcore Technique,” Biochemistry 36: 5072-5077 (1997); Meyers, Robert A., ed., Molecular Biology and Biotechnology. New York: Chernow Editorial Services, 1995; Christensen, Ulla, et al., “Stopped-flow kinetics of locked nucleic acid (LNA)-oligonucleotide duplex formation: studies of LNA-DNA and DNA-DNA interactions,” Biochem. J. 354: 481-484 (2001); Higuchi, H et al., “Enzymic synthesis of oligonucleotides containing methylphosphonate internucleotide linkages,” Biochemistry 29(37): 8747-53 (1990); Harrison, Joseph G., et al., “Screening for oligonucleotide binding affinity by a convenient fluorescence competition assay, ” Nucleic Acids Research 27(17): e14 i-v (1999); Prakash, Thazha P., et al., 2′O-{2-[N,N-(Dialkyl)aminooxy]ethyl}-Modified Antisense Oligonucleotides,” Organic Letters 2(25): 3995-3998 (2000); and Eriksson, Magdalena, and Nielsen, Peter E., “PNA-nucleic acid complexes. Structure, stability and dynamics,” Quarterly Reviews of Biophysics 29(4): 369-394 (1996); U.S. Pat. No. 5,539,083 Jul. 23, 1996

Cook, et al., “Peptide Nucleic Acid Combinational Libraries and Improved Methods of Synthesis”. U.S. Pat. No. 5,864,010 Jan. 26, 1999 Cook, et al., “Peptide Nucleic Acid Combinational Libraries”; U.S. Pat. No. 6,165,720 Dec. 26, 2000 Felgner et al., “Chemical Modification of DNA Using Peptide Nucleic Acid Conjugates”; U.S. Pat. No. 6, 201, 103 B1 Mar. 13, 2001 Nielsen, Et al., “Peptide Nucleic Acid Incorporating a Chiral Backbone”; U.S. Pat. No. 6,180,767 B1 Jan. 30, 2001 Wickstrom, et al., “Peptide Nucleic Acid Conjugates”; and U.S. Pat. No. 5,986,053 Nov. 16, 1999 Ecker, et al., “Peptide Nucleic Acids Complexes of Two Peptide Nucleic Acid Strands and One Nucleic Acid Strand”.; Liu G, Mang'era K, Liu N, Gupta S, Rusckowski M, Hnatowich D J. “Tumor pretargeting in mice using (99 m)Tc-labeled morpholino, a DNA analog”. J Nucl Med. 2002 43(3):384-91; and Wang Y, Chang F, Zhang Y, Liu N, Liu G, Gupta S, Rusckowski M, Hnatowich D J. “Pretargeting with amplification using polymeric peptide nucleic acid.”; Bioconjug Chem. 2001 (5):807-16; the contents of which are incorporated herein by reference in their entirety.

In preferred embodiments the bases of the oligo or polynucleotides are adenine, guanine, cytosine, thymine, and uracil. A large number of modified bases and purine and pyrimidine analogs that are also able to engage in base pairing are well known to one skilled in the arts and can also be employed.

In a preferred embodiment F(x) is a group that can bind specifically and with high affinity to two groups of M(x). In a preferred embodiment F(x) and M(x) are oligo or poly-nucleotides or analogs thereof that can form a Triplex struture comprised of 2 groups M(x) and one group F(x). Oligo and polynucleotides and analogs that can form triplexes are well known to one skilled in the arts and are described in Plum, G. Eric, et al. “Nucleic Acid Hybridization: Triplex Stability and Energetics,” Annu. Rev. Biophys. Biomol. Struct. 24:319-50 (1995); and

Frank-Kamenetskii, Maxim D., Mirkin, Sergei M., “Triplex DNA Structures,” Annu. Rev. Biochem 64:65-95 (1995) the contents of which are incorporated herein by reference in their entirety.

In preferred embodiments F(x) and f(k) are: 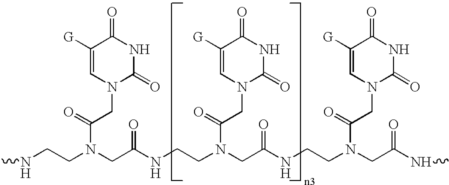
and M(x) and m(k) are: 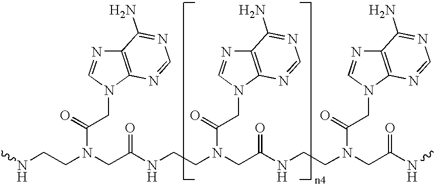
wherein G is H, or methyl, and wherein n3=2,3,4,5,6,7,8,9,10,11,12,13,14, 15,16,17,18,19,20,21,22,23 ,24,25, or about 25; and wherein n4=2,3,4,5,6,7, 8,9,10,11,12,13,14,15,16,17,18,19,20,21,22,23,24,25, or about 25; and wherein the wavy lines are ther sites of linker attachment, or the sites of trigger attachment, or H, or an inert group wherein the inert group is a group that does not impair the binding of F(x) and M(x).

In preferred embodiments F(x) and f(k) are: 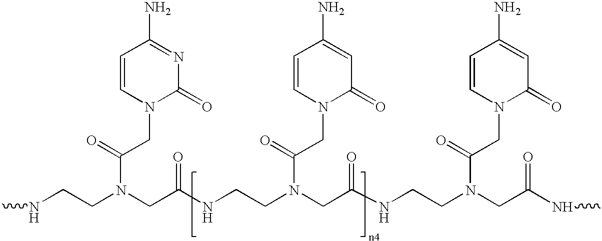
and M(x) and m(k) are: 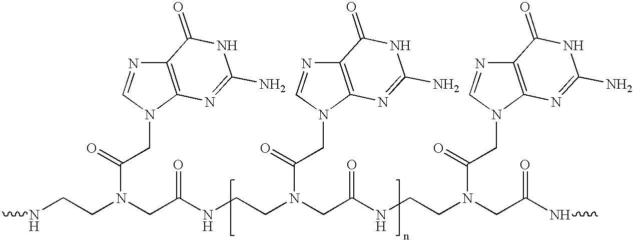
wherein n4=2,3,4,5,6,7,8,9,10,11,12,13,14,15,16,17,18,19,20,21,22, 23,24,25, or about 25; and wherein the wavy lines are the sites of linker attachment, or the sites of trigger attachment, or H, or an inert group; wherein the inert group is a group that does not impair the binding of F(x) and M(x).

In preferred embodiments the above F(x) and M(x) groups are interchanged.

Vancomycin and-D-alanine-D-Alanine Based Groups

In a preferred embodiment f(k) an m(k) are a vancomycin binding peptide and vancomycin. In a preferred embodiment the vancomycin binding peptide is comprised of D-alanine-D-alanine. In a preferred embodiment f(k) has the following structure: 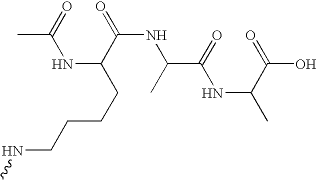
wherein the configuration of the lysine residue is L, and the alanines are D; and wherein the wavy line is the site of linker attachment; and m(k) has the following structure: 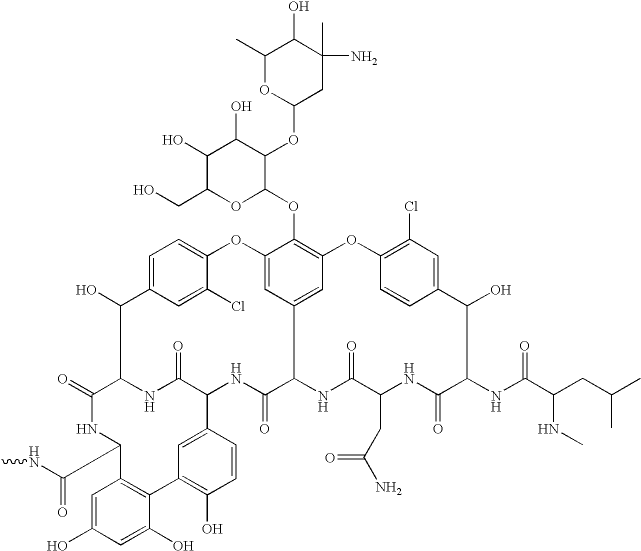
wherein the stereochemistry is as described for vancomycin and wherein the wavy line is the site of linker attachment.

In a preferred embodiment F(x) is comprised of a trimer of D-alanine-D-Alanine and M(x) is comprised of a trimer of vancomycin. This is based on the extraordinary affinity between trimeric vancomycin and trimeric d-Ala-d-Ala which has a dissociation constant of approximately 4×10⁻¹⁷ M as detailed by Rao, Jianghong, et al. “Design, Synthesis, and Characterization of a High-Affinity Trivalent System Derived from Vancomycin and L-Lys-D-Ala-D-Ala,” J. Am. Chem. Soc. 122: 2698-2710 (2000); and Rao, Jianghong, et al. “A Trivalent System from Vancomycin D-Ala-D-Ala with Higher Affinity Than Avidin Biotin,” Science 280 (1 May 1998); the contents of which are incorporated herein by reference in their entirety.

In a preferred embodiment F(x) has the following structure: 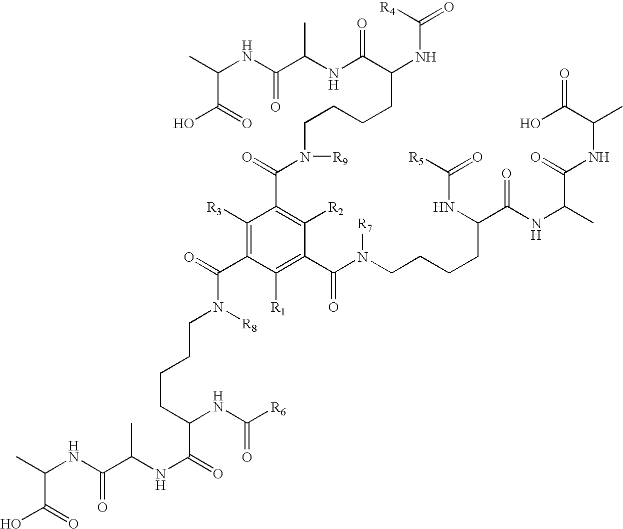
wherein the alanine residues are D configuration the lysine residues are the L configuration, and wherein R1,R2,R3,R7,R8,R9 are H or a site of linker attachment; and wherein R4,R5,R6 is methyl or a site of linker attachment;
and M(x) has the following structure: 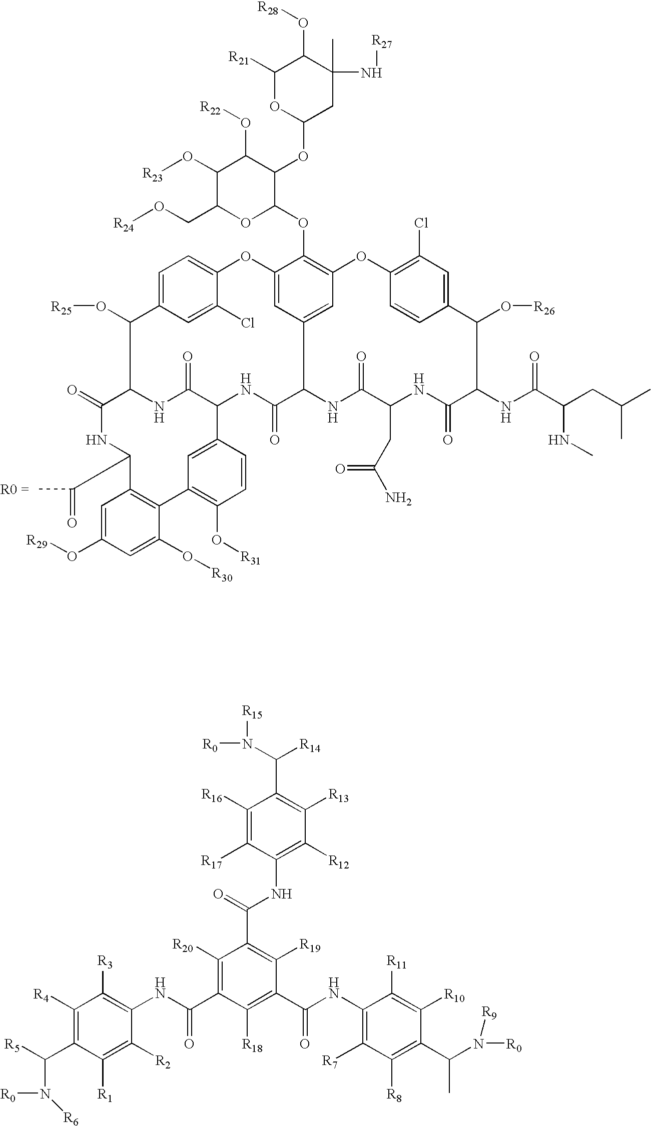
wherein R1-R31 is H; or a site of linker attachment. The solubility of the compound can be manipulated by varying substituents on the benzene rings.

In preferred embodiments R1, R2, R3, R4. R7. R8. R10, R11, R12, R13, R16, R17, R18, R19, and R20 can be OH, Cl, CO2H, NH₂, SO3H, —P(O)(OH)2, -phosphate, methyl, or a lower alkyl group, O-methyl, In a preferred embodiment one R27 is a site of linker attachment, and the remainder of the groups R are H. In a preferred embodiment one R22 is a site of linker attachment, and the remainder of the groups are H. In a preferred embodiment one R23 is a site of linker attachment, and the remainder of the groups R are H. In a preferred embodiment one R24 is a site of linker attachment, and the remainder of the groups R are H.

In a preferred embodiment F(x) has the following structure: 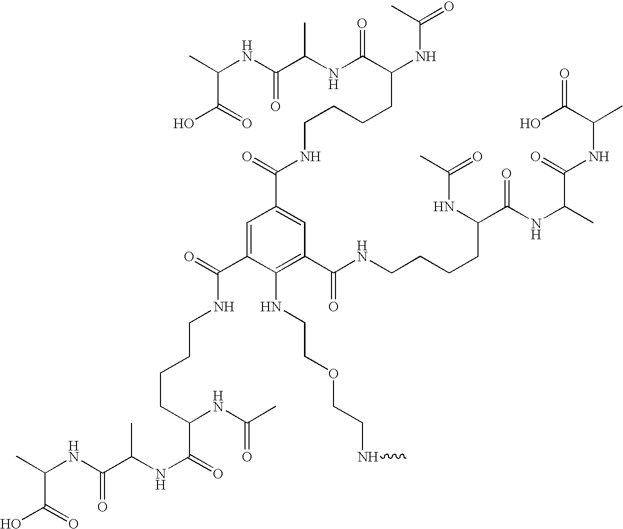
and M(x) has the following structure: 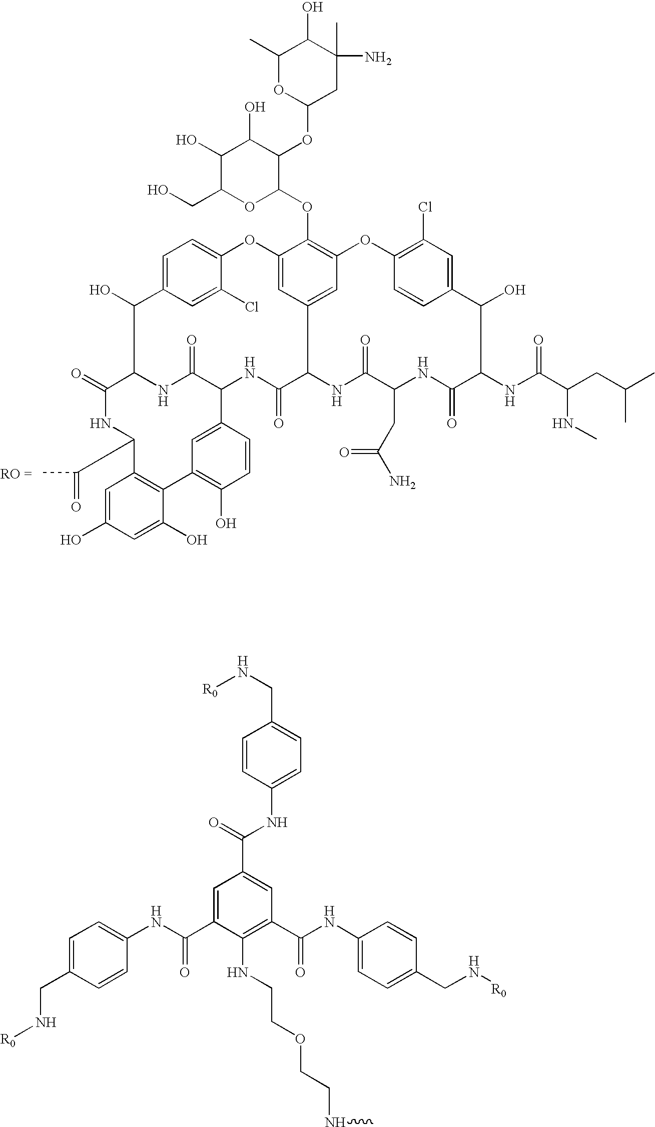
wherein the wavy lines are the site of linker attachment;

or M(x) has the following structure: 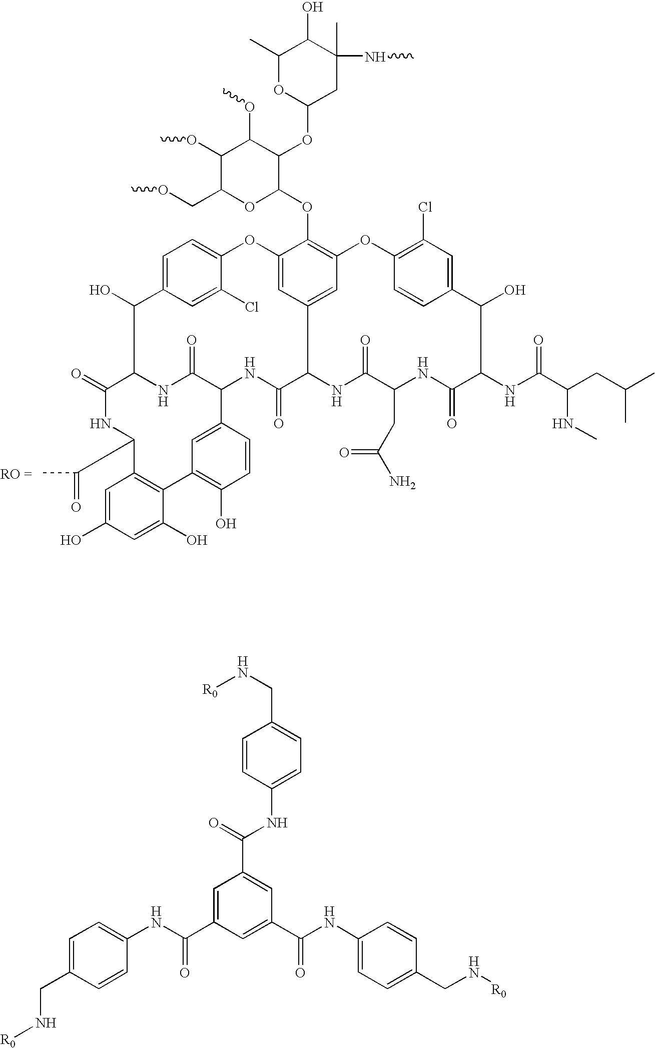
wherein the way line is H, or site of linker attachment to the remainder of the drug.

In a preferred embodiment F(x) has the following structure:

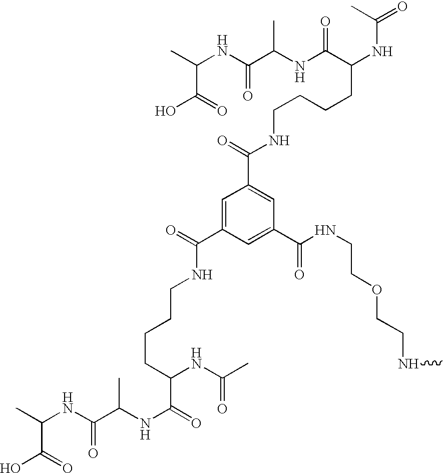

And M(x) has the following structure: 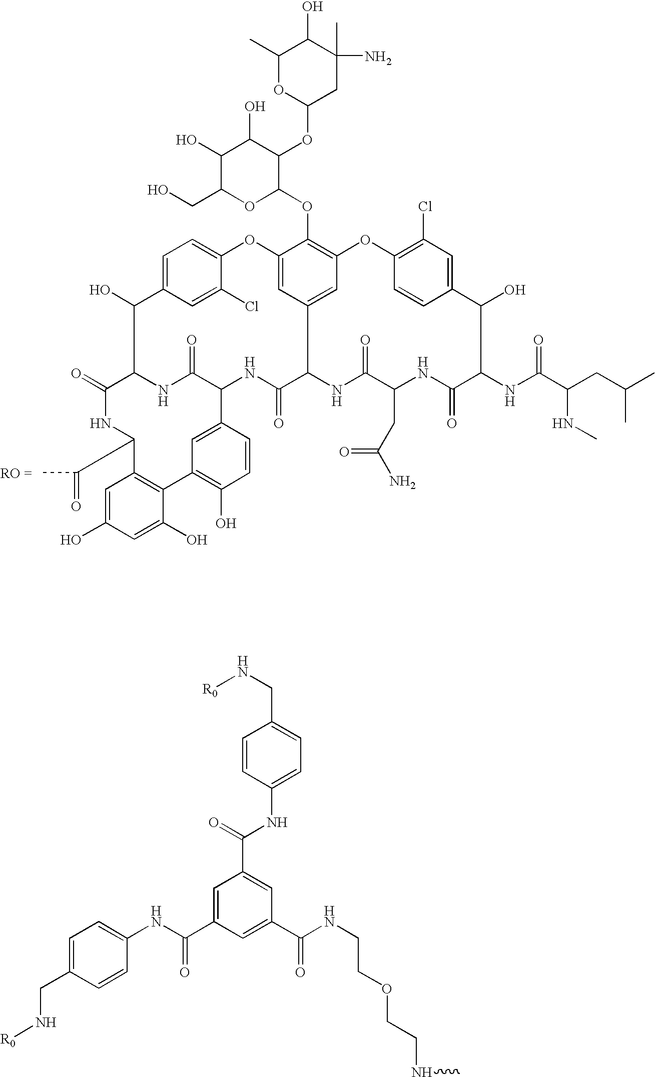
wherein the wavy lines are the sites of linker attachment.
pF(x) and Triggers

The groups designated as “pF(x)” and pf(k) are masked forms of the adaptors F(x) and f(k) which when unmasked are converted into F(x) and f(k) respectively and wherein the masked groups have decreased binding affinity to the ligands M(x) and m(k) respectively. Bioconversion of the masked female adaptor into the unmasked female adaptor can be by target selective or nonselective processes. In a preferred embodiment the unmasking is mediated by factors or biomolecules that are enriched at the target site or in the microenvironment of the target site. In a preferred embodiment the masked female adaptor is comprised of a receptor F(x) or f(k) to which is covalently attached a trigger group wherein the trigger group is located in such a position as to interfere with binding to M(x) or m(x). Trigger groups which can undergo bioreversible cleavage are well known to one skilled in the arts. A large number of suitable trigger groups and references related to this matter are described in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs”. Triggers that rapidly result in receptor unmasking upon activation are preferred. Preferred groups on F(x) or f(k) to which trigger groups can be attached include: NH2; secondary amino groups, tertiary amino groups; OH; CO2H; SH; phosphate, phosphate diester groups; phosphonate mono and diester groups; and phosphinate groups. In preferred embodiments the unmasking proceeds directly by an enzyme activated process or by an enzyme activated process that proceeds by the intermediacy of fleeting a very short lived or intermediate. Since the magnitude of the amplification is influenced by the number of amplification cycles it is desirable to employ groups that can be rapidly unmasked.

In a preferred embodiment the trigger can be activated by an enzyme that is delivered to the target cell via independently selective mechanisms. There have been intense efforts towards the development of tumor-selective antibodies coupled to enzymes to selectively activate prodrugs. A significant limitation with Antibody Directed Enzyme Prodrug Therapy (ADEPT), and related approaches is the requirement that for the targeted enzyme to efficiently activate the prodrug, the prodrug can be given at a concentration near the Michaelis Menton constant (Km) for the enzyme substrate interaction which is generally micromolar. Since all drugs are expected to have multiple pathways of metabolism, prodrug activation by non-targeted enzyme mechanisms can result in dose limiting toxicity. In the current approach systemic nontarget site trigger activation by the targeted enzyme can be inconsequential because of the extremely low concentrations of both the targeted enzyme and the targeted drugs. For those embodiments with a Compound 2 in which intramolecular binding between the male and female ligands can occur, optimal amplification will result only if the molecule is pre-bound to the target by the male ligand. In addition, the high effective concentration of the targeted enzyme and the targeted drugs at the targeted site can enable efficient trigger activation at the target cell. In addition to monoclonal antibodies- enzyme conjugates a target binding agent with a triggering enzyme attached can be employed. The enzyme can be targeted to a receptor on the target cell or in the microenvironment of the target cell or to a pattern of receptors as described in Ser. No. 09/712,465

Nov. 15, 2000 Glazier, Arnold. “Selective Cellular Targeting: Multifunctional Delivery Vehicles, Multifunctional Prodrugs, Use as Neoplastic Drugs” the contents of which are incorporated herein by reference in their entirety.

In a preferred embodiment an enzyme that can trigger the unmasking of F(x) or f(k) is coupled directly or by a linker to M(x). Targeted-enzyme conjugates and triggers that are suitable for use in ADEPT are well known to one in the arts can readily be adapted to the present invention. Procedures for coupling groups to enzymes and proteins are well known to one skilled in the arts and are detailed in Hermanson Greg T. (1996) “Bioconjugate Techniques.” Academic Press, Inc.; the contents of which are incorporated herein by reference in their entirety.

In a preferred embodiment the masked female adaptor is unmasked by a triggering enzyme that is enriched at the surface of tumor cells or in the microenvironment of tumor cells. In preferred embodiments the masked female adaptor is selected such that it can be unmasked by one of the following enzymes:
1.) Urokinase
2.) Plasmin
3.) Thrombin
4.) Activated factor VII
5.) Activated factor X
6.) Seprase
7.) Fibroblast activation protein
8.) Tissue plasminogen activator
9.) A matrix metalloproteinase (MMP)
10.) A membrane type matrix metalloproteinase
11.) A collagenase
12.) A gelatinase
13.) MMP-1; MMP-2; MMP-3; MMP-7; MMP-8; MMP-9; MMP-10; MMP-11; MMP-12; MMP-13; MMP-26
14.) MT-MMP-1, MT-MMP-2; MT-MMP-3; MT-MMP-4, MT-MMP-5; MT-MMP-6
15.) Prostate Specific Antigen (PSA)
16.) Prostate specific membrane antigen (PSMA)
17.) Human glandular kallikrein 2
18.) Human glandular Kallikrein 4
19.) Matripase
20.) Trypsin
21.) Guanidinobenzoatase
22.) Heparanase
23.) A cathepsin
24.) A cathepsin
25.) Cathepsins B; D; K; L; O; or S
26.) dipeptidyl peptidase IV
27.) gamma-glutamyl transpeptidase
28.) hepsin
29.) neutral endopeptidase
30.) pepsin c
31.) placental alkaline phosphatase
32.) acid phosphatase
33.) prostatic acid phosphatase
34.) stratum corneum chymotryptic enzyme
35.) SP220K
36.) sucrase-isomaltase
37.) TMPRSS2
38.) A type IV collagenase
39.) Prostase
40.) Aminopeptidase N
41.) Neutrophil elastase
42.) Membrane-type serine protease 1 (MT-SP1)
43.) TMPRSS4

In a preferred embodiment the group pF(x) or pf(k) is comprised of F(x) or f(k) respectively coupled to a trigger that is comprised of a substituted benzylic analog with a masked or latent electron donating group in the ortho or para positions. Unmasking of this group triggers cleavage of the bond between the benzylic carbon and a leaving group on F(x) or f(k). For a detailed discussion of this type of trigger see: Carl, P., “A Novel Connector Linkage Applicable in Prodrug Design,” J Med Chem, 24(5):479-480 (1981); U.S. Pat. No. 5,627,165, May 6, 1997, Glazier, “Phosphorous Prodrugs and Therapeutic Delivery Systems Using Same”; U.S. Pat. No. 5,274,162, Dec. 28, 1993, Glazier, “Antineoplastic Drugs with Bipolar Toxification/Detoxification Functionalities”; U.S. Pat. No. 5,659,061, Aug. 19, 1997, Glazier, “Tumor Protease Activated Prodrugs of Phosphoramide Mustard Analogs with Toxification and Detoxification Functionalities”; Senter, Peter D., et al., “Development of a Drug-Release Strategy Based on the Reductive Fragmentation of Benzyl Carbamate Disulfides,” J Org Chem, 55:2975-2978 (1990), the contents of which are incorporated herein by reference in their entirety.

Note: For the sake of clarity the trigger groups shown in this section include an attached moiety “Y” that is released upon trigger activation or trigger function. Strictly speaking, the released group Y is not part of the trigger group.

In a preferred embodiment the trigger p has the following structure: 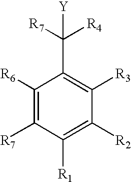
wherein Y is the leaving group; and R₁ and R₃, either alone or both, are groups which can be transformed into electron donating groups, and wherein R₁, R₂, R₃, R₄, R₅, R₆, and R₇ can be hydrogen, alkyl groups, halogens, alkoxy, —CO—R₈, where R₈ is OH, an alkyl alkoxy group, or where R₈ can be such that COR₈ comprises an amide. At least one of the groups R₁ and R₃ must be capable of transformation or bio-transformation into an electron donating group. R₁ and R₃ can be an ester, amide, thioester, disulfide, nitro group, H, azido, phosphoester, phosphonoester, phosphinoester, sulfate, alkoxy group, an amino group that is phosphonylated, or phosphorylated and enol ether, an acetal group, a carbonate, or a carbamate.

In a preferred embodiment the groups R1 or R3 above are converted into an electron donating group by the action of a triggering enzyme that is enriched on the target cell or in the microenvironment of the target cell. In a preferred embodiment R1 or R3 are amide groups that can be selectively cleaved by the triggering enzyme. In a preferred embodiment the trigger has the following structure: 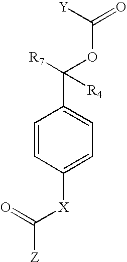
wherein the group X is NH, O, or S; and R4 and R7 are H, or methyl; and Y is —NH; or derived from a secondary amino group on the group F(x) or f(k); and wherein Z is a group selected such that the triggering enzyme enriched at the target site can cleave the resulting amide, ester, or thioester and unmask an electron donating group that in turn can trigger cleavage of the benzylic C—O bond and free YH. One skilled in the arts will recognize numerous groups Z that confer specificity for particular enzymes. In addition methods are well known to allow the facile identification of groups Z that confer substrate specificity for an enzyme The following references relate to this matter Harris J L, Backes B J, Leonetti F, Mahrus S, Ellman J A, Craik C S; “Rapid and general profiling of protease specificity by using combinatorial fluorogenic substrate libraries” Proc Natl Acad Sci USA (2000);97(14):7754-9. , Lien S, Francis G L, Graham L D; “Combinatorial strategies for the discovery of novel protease specificities”; Comb Chem High Throughput Screen. (1999) (2):73-90; and McDonald, J. K., and Barrett, A. J. Mammalian Proteases: A Glossary and Bibliography. Vol. 2: Exopeptidases. Orlando, Fla.: Academic Press, Inc., 1986; the contents of which are incorporated herein by reference in their entirety.

In a preferred embodiments pF(x) and pf(k) are oligo or poly-nucleotides or analogs thereof, wherein one or more of the bases are modified in a bioreversible manner such as to preclude or impair base pairing with the complementary M(x) or m(k) strand. In a preferred embodiment an amino group of the base is converted into a bio-reversible carbamate group. In a preferred embodiment an amino group of the base is methylated and also converted into a bio-reversible carbamate group. In a preferred embodiment one or more bases of the oligo or poly-nucleotide or analog thereof has the following structure: 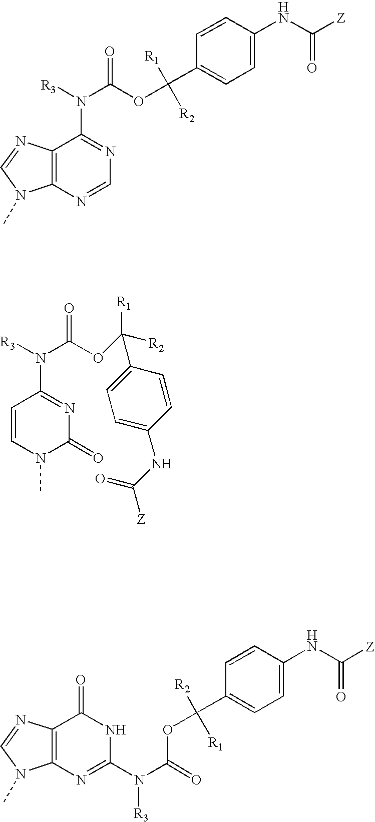
wherein the dotted line is the site of base attachment to the remainder of the oligo or poly-nucleotide; and wherein R3 is H, CH3, or a lower alkyl group; or a bioreversible masking group; and R1, and R2 are H, of methyl, or a lower alkyl group, and wherein Z is selected such that the resulting amide can be cleaved by an enzyme enriched at the target site; and wherein R3 can also be a group of the following structure: 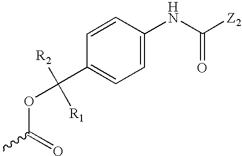
wherein the wavy line is the site of attachment; and wherein Z2 is a group such that the resulting amide can be cleaved by an enzyme enriched at the target site; and wherein Z1 and Z2 may be the same or different groups.

In preferred embodiments wherein Z-C(O)OH is an amino acid, or an oligo-peptide comprised of between 2 and about 25 amino acids; or analogs thereof. In preferred embodiments Z1-C(O)— and Z2-C(O)— are selected from the following structures that are preferentially cleaved by plasmin:

D-Val-Leu-Lys-
and;
Acetyl-Lys-Thr-Tyr-Lys-
and;
Acetyl-Lys-Thr-Phe-Lys-
and;
Acetyl-Lys-Thr-Trp-Lys-
and;

wherein the carboxy group of the lysine residue is the site of attachment;

and the following structures that are preferentially cleaved by urokinase:

H-glutamyl-glycyl-L-arg- and;
pyro-glutamyl-glycyl-L-arg- and;
H-D-isoleucyl-L-prolyl-L-arg;

wherein the carboxy group of the arginine is the site of attachment;

and the following structure which is cleaved by human glandular kallikrein 2:

Pro-Phe-Arg-
and;
Ala-Arg-ArG-;

wherein the carboxy group of the arginine is the site of attachment;

and the following structure which is cleaved by PSA:

His-Ser-Ser-Lys-Leu-Gln-
and;
N-Glutaryl-(4-hydroxypropyl)Ala-Ser-
Cyclohexaglycyl-Gln-Ser-Leu-;

Wherein the site of attachment is at the carboxy group of the GLn and the Leu respectively;

and the following structures which are cleaved by the enzyme matriptase:

Boc-Gln-Ala-Arg-
and;
Boc-benzyl-Glu-Gly-Arg-
and;
Boc-Leu-Gly-Arg-
and;
Boc-benzyl-Asp-Pro-Arg-
and;
Boc-Phe-Ser-Arg-
and;
Boc-Val-Pro-Arg-
and;
Boc-Leu-Arg-Arg-;
and;
Boc-Gly-Lys-Arg-and;,
and
Boc-Leu-Ser-Thr-Arg-;

wherein the C terminal carboxyl group is the site of attachment.

The following references relate to this subject matter:

Backes B J, et al. “Synthesis of positional-scanning libraries of fluorogenic peptide substrates to define the extended substrate specificity of plasmin and thrombin,” Nat Biotechnol 18(2):187-93 (2000); Cavallaro, Gennara, et al. “Polymeric Prodrug for Release of an Antitumoral Agent by Specific Enzymes,” Bioconjugate Chem 12: 143-151 2001; Liu, Shihui, et al. “Targeting of Tumor Cells by Cell Surface Urokinase Plasminogen Activator-dependent Anthrax Toxin,” J. Biol. Chem., 276(21):17976-17984, May 25, 2001; WO 01/09165 A2 Jul. 28, 2000 Denmeade, et al., “Activation of Peptide Prodrugs by hK2”; Mikolajczyk S D, et al., “Human glandular kallikrein, hK2, shows arginine-restricted specificity and forms complexes with plasma protease inhibitors,” Prostate 34(1):44-50 Jan. 1, 1998; Lin C Y, et al. “Molecular cloning of cDNA for matriptase, a matrix-degrading serine protease with trypsin-like activity,” J Biol Chem 274(26):18231-6 Jun. 25, 1999; Denmeade, Samuel R., et al. “Specific and Efficient Peptide Substrates for Assaying the Proteolytic Activity of Prostate-specific Antigen,” Cancer Research 57:4924-4930 Nov. 1, 1997; Denmeade, Samuel R., Isaacs, John T. “Enzymatic Activation of Prodrugs by Prostate-Specific Antigen: Targeted Therapy for Metastatic Prostate Cancer,” Cancer Journal Scientific American 4: S15-S211998; DeFeo-Jones, Deborah, et al. “A peptide-doxorubicin ‘prodrug’ activated by prostate-specific antigen selectively kills prostate tumor cells positive for prostate-specific antigen in vivo,” Nature Medicine 6(11):1248-1252 November 2000; Coombs, Gary S, et al. “Substrate specificity of prostate-specific antigen (PSA),” Chemistry & Biology 5:475-488 September 1998; the contents of which are incorporated herein by reference in their entirety.

Many tumor associated enzymes cleave internal bonds and do not efficiently cleave at terminal sites. A preferred type of masking group “p” to mask F(x) and f(k) and enable unmasking by enzymes with this substrate requirement is comprised of:
F(x)—S—B or f(k)—S—B
Wherein “S” is a substrate that can be cleaved by the triggering enzyme; and “B” is a group that prevents the binding of F(x) or f(k) to M(x) or m(k) respectively; and wherein cleavage of S by the trigger enzymes restores the ability of the F(x) or f(k) group to bind to M(x) or m(k) by liberating the B group. The groups may be directly connected or may be connected by a linkers. In another preferred embodiment F(x)—S is a cyclic structure that cannot bind to M(x). Cleavage of S opens the cycle and restores receptor binding function.

In a preferred embodiment F(x) or f(k) is an oligo or poly-nucleotide or analog thereof, and S is a oligo-peptide, and B is a complementary oligo-nucleotide or analog thereof that can bind in an intramolecular fashion to F(x) or f(k). Preferably B is a shorter oligo-nucleotide and therefore will have lower affinity than M(x) or m(k). In a preferred embodiment S is an oligo-peptide or analog thereof that is 3,4,5,6,7,8,9,10,11,1,2,1,3,14,1,5,1,6,17,18,19,20 or about 20 amino acids long.

One skilled in the arts will recognize or be able to ascertain using well known routine methodologies a large number of groups “S” that are selectively cleaved by enzymes that are enriched at tumor or target cells. The following references relate to this matter: Barrett, A. J., and McDonald, J. K. Mammalian Proteases: A Glossary and Bibliography. Vol. 1: Endopeptidases. New York. Academic Press, Inc., 1980; Butenas S, et al. “Analysis of tissue plasminogen activator specificity using peptidyl fluorogenic substrates,” Biochemistry 36(8):2123-31, Feb. 25, 1997; Peterson J J, Meares C F. “Cathepsin substrates as cleavable peptide linkers in bioconjugates, selected from a fluorescence quench combinatorial library,” Bioconjug Chem 9(5):618-26 September-October 1998; Yasuda Y, et al. “Characterization of new fluorogenic substrates for the rapid and sensitive assay of cathepsin E and cathepsin D,” J Biochem (Tokyo) 125(6):1137-43 January 1999; “Combinatorial strategies for the discovery of novel protease specificities,” Comb Chem High Throughput Screen 2(2):73-90 April 1999; Netzel-Arnett S, et al. “Continuously recording fluorescent assays optimized for five human matrix metalloproteinases,” Anal Biochem 195(1):86-92 May 15, 1991; Grahn S, et al. “Design and synthesis of fluorogenic trypsin peptide substrates based on resonance energy transfer,” Anal Biochem 265(2):225-31 Dec. 15, 1998; Yang C F, et al. “Design of synthetic hexapeptide substrates for prostate-specific antigen using single-position minilibraries,” J Pept Res 54(5):444-8 November 1999; Beekman B, et al. “Fluorogenic MMP activity assay for plasma including MMPs complexed to alpha 2-macroglobulin,” Ann N Y Acad Sci878:150-8 Jun. 30, 1999; Beekman B, et al. “Highly increased levels of active stromelysin in rheumatoid synovial fluid determined by a selective fluorogenic assay,” FEBS Lett418(3):305-9 Dec. 1, 1997; Mikolajczyk S D, et al.; Ohkubo S, et al. “Identification of substrate sequences for membrane type-1 matrix metalloproteinase using bacteriophage peptide display library,” Biochem Biophys Res Commun 266(2):308-13 Dec. 20, 1999; Tung C H, et al. “In vivo imaging of proteolytic enzyme activity using a novel molecular reporter,” Cancer Res 60(17):4953-8 Sep. 1, 2000; Mucha A, et al. “Membrane type-1 matrix metalloprotease and stromelysin-3 cleave more efficiently synthetic substrates containing unusual amino acids in their P1′ positions,” J Biol Chem 273(5):2763-8 Jan. 30, 1998; Bianco A, et al. “N-hydroxy peptides as substrates for alpha-chymotrypsin,” J Pept Res 54(6):544-8 December 1999; Tung C H, et al., “Preparation of a cathepsin D sensitive near-infrared fluorescence probe for imaging,” Bioconjug Chem 10(5):892-6 September-October 1999; Harris J L, et al. “Rapid and general profiling of protease specificity by using combinatorial fluorogenic substrate libraries,” Proc Natl Acad Sci USA 97(14):7754-9 Jul. 5, 2000; Ottl J, et al. “Recognition and catabolism of synthetic heterotrimeric collagen peptides by matrix metalloproteinases,” Chem Biol 7(2):119-32 February 2000;; Deng S J, et al. “Substrate specificity of human collagenase 3 assessed using a phage-displayed peptide library,” J Biol Chem 275(40):31422-7 Oct. 6, 2000; Edwards P D, et al. “Backes B J, et al. “Synthesis of positional-scanning libraries of fluorogenic peptide substrates to define the extended substrate specificity of plasmin and thrombin,” Nat Biotechnol 18(2):187-93 February 2000; and Hervio L S, et al. “Negative selectivity and the evolution of protease cascades: the specificity of plasmin for peptide and protein substrates,” Chem Biol 7(6):443-53 June 2000; the contents of which are incorporated herein by reference in their entirety.

In preferred embodiments pF(x) and pf(k) are: 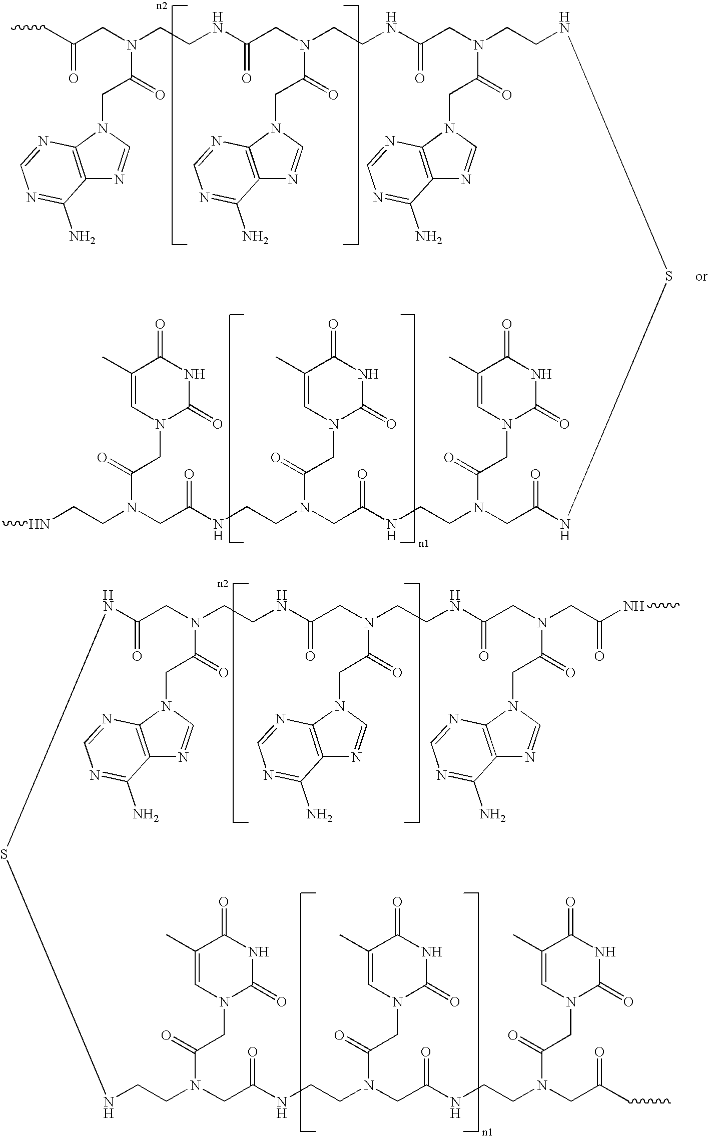 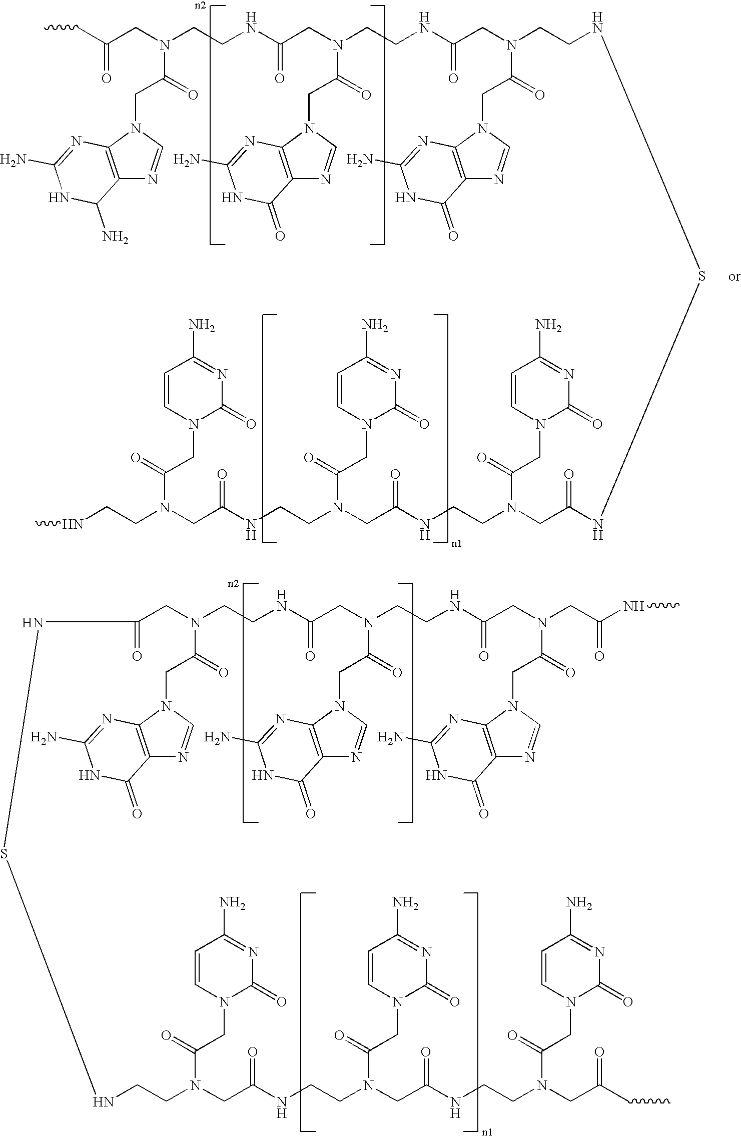
wherein n1=2,3,4,5,6,7,8,9,10,11,12,13,14,15,16,17,18,19, or about 20; and
wherein n2=2,3,4,5,6,7,8,9,10,11,12,13,14,15,16,17,18,19, or about 20, and
wherein one of the wavy lines is the sites of linker attachment, and the other wavy line is or H; OH, or an inert group wherein the inert group is a group that does not impair the binding of F(x) and M(x); and wherein the group “S” is comprised of an oligo-peptide that can be cleaved by a triggering enzyme that is enriched at the target cell or tumor cell. In a preferred embodiment if the site of linker attachment to the remainder of the targeted drug is the thymidine bearing side than n1 is greater than n2. In a preferred embodiment if the adenine bearing side is the site of linker attachment to the remainder of the targeted drug than n2 is greater than n1. In a preferred embodiment if the site of linker attachment to the remainder of the targeted drug is the cytidine bearing side than n1 is greater than n2. In a preferred embodiment if the guanine bearing side is the site of linker attachment to the remainder of the targeted drug than n2 is greater than n1.

In preferred embodiments the triggering enzyme is MMP-2; MMP-9 or membrane-type 1 MMP (MT1-MMP) and “S” is comprised of:

Gly-pro-leu-gly-met-leu-ser-gln-; or
Gly-pro-leu-gly-leu-trp-ala-gln- or
Gly-pro-leu-gly-leu-arg-ser-trp- or
Gly-pro-leu-pro-leu-arg-ser-trp- or
Pro-leu-ala-cys(O-methyl-benzyl)-trp-ala-arg-

wherein the cysteine is substituted at the sulfur, as indicated with a p-methoxybenzyl group.

In preferred embodiments the triggering enzyme is urokinase ans S is comprised of: Pro-gly-ser-gly-lys-ser-ala-.

In preferred embodiments the triggering enzyme is plasmin and S is comprised of :Leu-ly-gly-ser-gly-ile-tyr-arg-ser-arg-ser-leu-glu-.

In preferred embodiments the triggering enzyme is PSA and S is comprised of:

Gly-ile-ser-ser-phe-tyr-ser-ser-thr-glu-glu-leu-
trp-
or
Ser-ser-ile-tyr-ser-gln-thr-glu-glu-gln

In preferred embodiments the triggering enzyme is MMP-13 and S is comprised of:

Gly-pro-leu-gly-met-arg-gly-leu-
or
Gly-pro-leu-gly-leu-trp-ala-arg-
or
Gly-pro-arg-pro-phe-Asn-tyr-leu-
or

In preferred embodiments the triggering enzyme is MMP-9 and S is comprised of:

Ser-gly-lys-gly-pro-arg-gln-ile-thr-ala-
or
Ser-gly-lys-ile-pro-arg-arg-leu-thr-ala-.

The following references relate to this matter: Liu, Shihui, et al. “Tumor Cell-selective Cytotoxicity of Matrix Metalloproteinase-activated Anthrax Toxin,” Cancer Research 60, 6061-6067,, (2000); Hervio L S, et al. “Negative selectivity and the evolution of protease cascades: the specificity of plasmin for peptide and protein substrates,” Chem Biol 7(6):443-53 June 2000; Mikolajczyk SD, et al.; Ohkubo S, et al. “Identification of substrate sequences for membrane type-1 matrix metalloproteinase using bacteriophage peptide display library,” Biochem Biophys Res Commun 266(2):308-13 Dec. 20, 1999; Mucha A, et al. “Membrane type-1 matrix metalloprotease and stromelysin-3 cleave more efficiently synthetic substrates containing unusual amino acids in their P1′ positions,” J Biol Chem 273(5):2763-8, (1998); Deng S J, et al. “Substrate specificity of human collagenase 3 assessed using a phage-displayed peptide library,” J Biol Chem 275(40):31422-7 Oct. 6, 2000; Kridel, Steven J., et al. “Substrate Hydrolysis by Matrix Metalloproteinase-9,” Journal of Biological Chemistry 276(23):20572-8 (2001); Liu, Shihui, et al. “Targeting of Tumor Cells by Cell Surface Urokinase Plasminogen Activator-dependent Anthrax Toxin,” J. Biol. Chem., 276(21):17976-17984, May 25, 2001; and Coombs, Gary S, et al. “Substrate specificity of prostate-specific antigen (PSA),” Chemistry & Biology 5:475488 (1998); and Rehault S, Brillard-Bourdet M, Bourgeois L, Frenette G, Juliano L, Gauthier F, Moreau T.; “Design of new and sensitive fluorogenic substrates for human kallikrein hK3 (prostate-specific antigen) derived from semenogelin sequences.” Biochim Biophys Acta. 2002 596(1):55-62; the contents of which are incorporated herein by reference in their entirety.

In a preferred embodiment pf(k) is comprised of a group of the following structure: 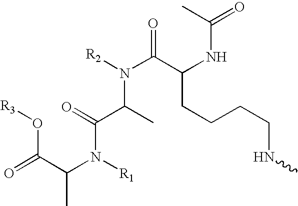
wherein the alanines are the D configuration, and wherein R1 R2, and R3 are H or bioreversible masking groups that can be removed by triggering enzymes that are enriched at the target cell; and the wavy line is the site of linker attachment.

In a preferred embodiment pf(k) has the following structure: 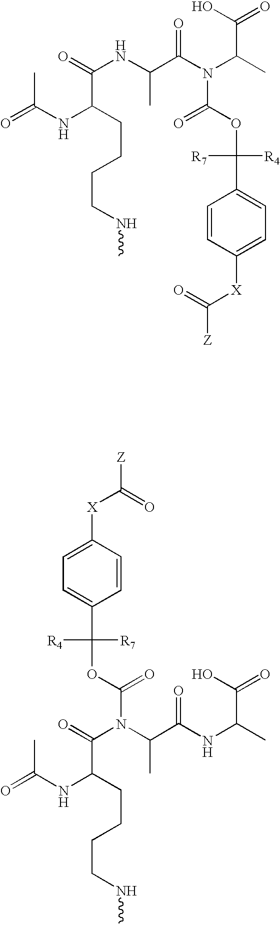
wherein the group X is NH, O, or S; and R4 and R7 are H, or methyl; and wherein Z is a group selected such that the triggering enzyme enriched at the target site can cleave the resulting amide, ester, or thioester.

In preferred embodiments Z-C(O)OH is an amino acid, or an oligo-peptide comprised of between 2 and about 25 amino acids; or analogs thereof. In preferred embodiments In preferred embodiments Z-C(O)— is selected from the following structures that are preferentially cleaved by plasmin:

D-Val-Leu-Lys-
and;
Acetyl-Lys-Thr-Tyr-Lys-
and;
Acetyl-Lys-Thr-Phe-Lys-
and;
Acetyl-Lys-Thr-Trp-Lys-
and;

wherein the carboxy group of the lysine residue is the site of attachment;

and the following structures that are preferentially cleaved by urokinase:

H-glutamyl-glycyl-L-arg- and;
pyro-glutamyl-glycyl-L-arg- and;
H-D-isoleucyl-L-prolyl-L-arg-;

wherein the carboxy group of the arginine is the site of attachment;

and the following structure which is cleaved by human glandular kallikrein 2:

Pro-Phe-Arg-
and;
Ala-Arg-ArG-;

wherein the carboxy group of the arginine is the site of attachment;

and the following structure which is cleaved by PSA:

His-Ser-Ser-Lys-Leu-Gln-
and;
N-Glutaryl-(4-hydroxypropyl)Ala-Ser-
Cyclohexaglycyl-Gln-Ser-Leu

Wherein the site of attachment is at the carboxy group of the GLn and the Leu respectively;

and the following structures which are cleaved by the enzyme matriptase:

Boc-Gln-Ala-Arg-
and;
Boc-benzyl-Glu-Gly-Arg-
and;
Boc-Leu-Gly-Arg-
and;
Boc-benzyl-Asp-Pro-Arg-
and;
Boc-Phe-Ser-Arg-
and;
Boc-Val-Pro-Arg-
and;
succinyl-Ala-Phe-Lys-
and,
Boc-Leu-Arg-Arg-;
and;
Boc-Gly-Lys-Arg-and;,
and
Boc-Leu-Ser-Thr-Arg-;

Wherein the C terminal carboxyl group is the site of attachment;

In preferred embodiments pF(x) has the following structures: 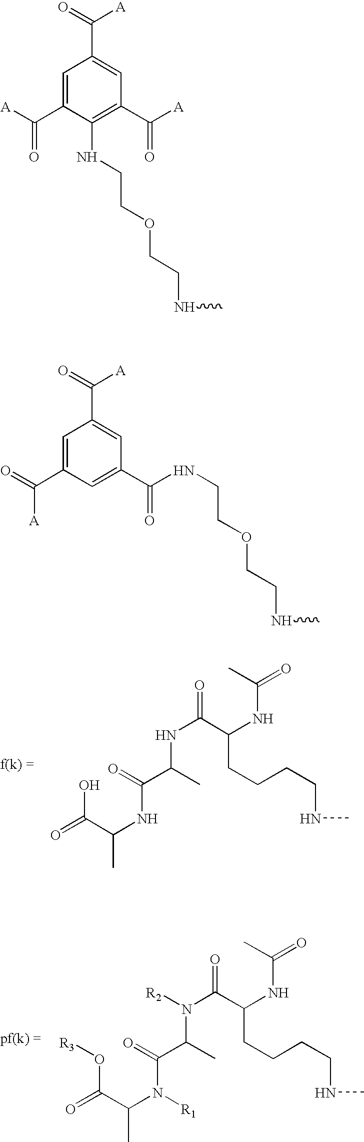
wherein “A” is the group f(k) or the group pf(k); and wherein at least one of the groups A is pf(k); and wherein the alanines are the D configuration, and wherein R1 and R2 are H or bioreversible masking groups that can be removed by triggering enzymes that are enriched at the target cell; and wherein R3 is OH or a or bioreversible masking groups that are removed by triggering enzymes that are enriched at the target cell; and the wavy line is the site of linker attachment, and wherein the dotted line is the site of attachment of pf(k). In preferred embodiments of the above pf(k) has the following structure:

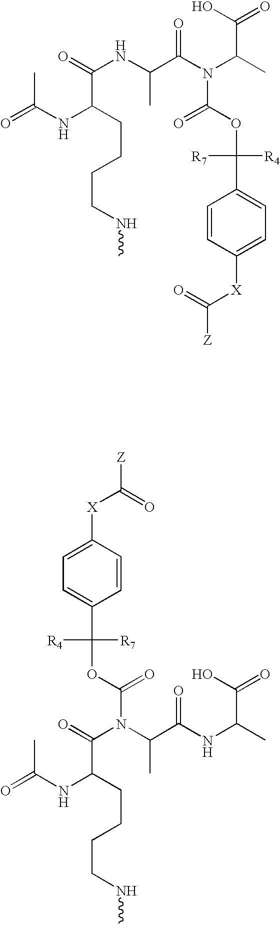

wherein the group X is NH, O, or S; and R4 and R7 are H, or methyl; and wherein Z is a group selected such that the triggering enzyme enriched at the target site can cleave the resulting amide, ester, or thioester. In preferred embodiments Z-C(O)OH is an amino acid, or an oligo-peptide comprised of between 2 and about 25 amino acids; or analogs thereof. In preferred embodiments Z-C(O)— is selected from

D-Val-Leu-Lys- and;
Acetyl-Lys-Thr-Tyr-Lys- and;
Acetyl-Lys-Thr-Phe-Lys- and;
Acetyl-Lys-Thr-Trp-Lys- and;
H-glutamyl-glycyl-L-arg- and;
pyro-glutamyl-glycyl-L-arg- and;
H-D-isoleucyl-L-prolyl-L-arg-;
Pro-Phe-Arg- and;
Ala-Arg-ArG-;
His-Ser-Ser-Lys-Leu-Gln- and;
N-Glutaryl-(4-hydroxypropyl)Ala-Ser-Cyclohexaglycyl-Gln-Ser-Leu-;
Boc-Gln-Ala-Arg- and;
Bocc-benzyl-Glu-Gly-Arg- and;
Boc-Leu-Gly-Arg- and;
Boc-benzyl-Asp-Pro-Arg- and;
Boc-Phe-Ser-Arg- and;
Boc-Val-Pro-Arg- and;
succinyl-Ala-Phe-Lys- and,
Boc-Leu-Arg-Arg-; and;
Boc-Gly-Lyss-Arg- and; ,and
Boc-Leu-Ser-Thr-Arg-;

Wherein the C terminal carboxyl group is the site of attachment.
Triggers to Release the Effector Agents

The manner of coupling of the effector agents to the remainder of the drug depends upon the required functionality. Some effector agents can evoke their desired effect while attached to the drug. Other effector agents have optimal activity when released. In a preferred embodiment the effector agent E is connected to the remainder of the drug by a trigger that when activated releases the effector agent from the remainder of the drug complex. This release may be intracellular or extracellular and can be mediated by a wide range of triggers. Numerous examples of preferred triggers are given in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs”. When activated the triggers can release the effector agents.

In a preferred embodiment, triggers undergo cleavage intracellularly and thereby release then free toxins. Intracellular triggers can be activated by a wide range of intracellular enzymes including: hydrolases, proteases, amidases, glycoside hydrolases, thioreductases, Glutathione-S-Transferases, nitroreductases, oxidases, phosphodiesterases, quinone reductases, phosphatases, thiolesterases, oxidoreductases, sulfatases, and esterases.

In a preferred embodiment the trigger is comprised of a substituted benzylic analog with a masked or latent electron-donating group in the ortho or para positions as described elsewhere in this document. Another preferred embodiment of the trigger utilizes a masked nucleophile which when unmasked catalyzes an intramolecular reaction. A preferred embodiment of a trigger is comprised of the following structure: 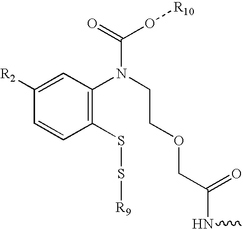
wherein R₂ is H, or a nitro group; R₉ is a group selected such that the resulting S—S bond can be reduced by cells to give the corresponding thiol; R₉ can be an alkyl or aryl group, which can bear substituents; and R₉ can be a cysteine or a derivative of cysteine. Substituents on R₉ can include amino, hydroxy, phosphonate, phosphate, or sulfate, which can serve to increase water solubility. Triggers of this class function by a rapid cyclization reaction due to the high effective molarity of the neighboring nucleophile. The following references relate to this subject matter: Hutchins J. E. C.; Fife T. H., “Fast Intramolecular Nucleophilic Attack by Phenoxide Ion on Carbamate Ester Groups,” J Am Chem Soc, 95(7):2282-2286 (1973); and Fife T. H., et al., “Highly Efficient Intramolecular Nucleophilic Reactions. The Cyclization of p-Nitrophenyl N-(2-Mercaptophenyl)-N-methylcarbamate and Phenyl N-(2-Aminophenyl)-N-methylcarbamate,” J Am Chem Soc, 97(20):5878-5882 (1975), the contents of which are incorporated herein by reference in their entirety.

Another preferred embodiment of an intracellular trigger, has the following structure:
wherein R₁ is a group such that the resulting S—S bond can be reduced by cells 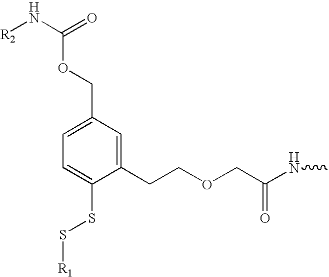
to give the corresponding thiol. R₁ can be a lower alkyl or aryl group, which can bear inert substituents. R₁ can be a cysteine or a derivative of cysteine. Substituents on R₁ can include: amino, hydroxy, phosphonate, phosphate, or sulfate groups that increase water solubility; and wherein R₂—NH₂ is the drug or molecule that is freed upon activation of the trigger; and wherein the wavy line is the site of a linker attachment to the remainder of the drug complex.

Another preferred embodiment of a trigger for use with effector agents that have adjacent hydroxy groups is shown below: 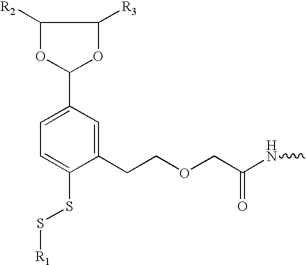
wherein R₁ is a group such that the resulting S—S bond can be reduced by cells to give the corresponding thiol. R₁ can be a lower alkyl or aryl group, which can bear inert substituents. R₁ can be a cysteine or a derivative of cysteine. Substituents on R₁ can include: amino, hydroxy, phosphonate, phosphate, or sulfate groups that increase water solubility; and wherein HO—R2-R3-OH is the drug or molecule that is freed upon activation of the trigger; and wherein the wavy line is the site of a linker attachment. The benzylic ring may also be substituted with inert substituents that do not interfere with the following mechanism of action: Reduction of the disulfide group unmasks a powerfully electron donating thiolate anion (Hammett Sigma+constant−2.62) that can trigger acetal hydrolysis by stabilization of carbocation formation at the benzylic carbon. The following references relate to this matter: Hansch, C.; Leo, A.; Hoekman, D.; in “Exploring QSAR Hydrophobic, Electronic and Steric Constants” ACS Professional Reference Book (1995); and Fife, T.; Jao, L.; “Substituent Effects in Acetal Hydrolysis”, J.Org. Chem.; p.1492; (1965); the contents of which are incorporated herein by reference in their entirety.

The above description gives numerous embodiments of the substituents and connections of the components: T, F(x), pF(x), M(x) E, triggers, linkers, and that can comprise the Compounds of the present invention. One skilled in the arts will recognize numerous other substituents that can comprise the components of the present invention and these are to be considered within the scope of the present invention.

Some preferred embodiments based on Vancomycin trimer and D-Ala-A-ala trimer:

A preferred embodiment of Compound 1 is comprised of:
T-L-F(x) or T-L-pF(x)

Wherein T is a targeting agent connected by a linker designated as “L” to a group F(x) comprised of a trimer of D-alanine-D-Alanine or a group pF(x) comprised of a masked trimer of D-alanine-D-Alanine.

In a preferred embodiment of the above Compound 1: T-L-pF(x) has the following structure: 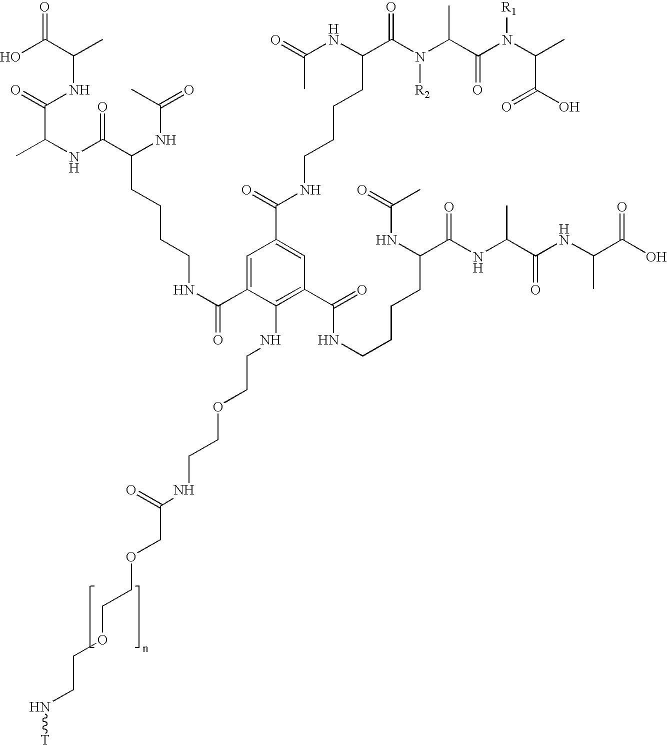
wherein n is 0,1,2,3,4,5,6,7,8,9,10, . . . 50, or about 50; and the wavy line is the site of linker attachment to T; and wherein R1 is H, or a bioreversible masking or trigger group, and wherein R2 is H, or a bioreversible masking or trigger group, and wherein R1 and R2 are not both H. In preferred embodiments the linker is connected to T by an amide, or carbamate group. In preferred embodiments n=10 and R1=H; and R2 has the following structure:

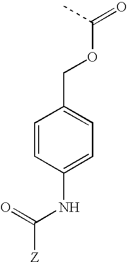

wherein Z is a group such that the resulting amide can be cleaved by an enzyme enriched at the target cell or in the microenvironment of the target cell.

In a preferred embodiment Z is selected such that the amide can be cleaved by a tumor associated protease. In preferred embodiments Z-C(O)— is selected from

D-Val-Leu-Lys- and;
Acetyl-Lyss-Thr-Tyr-Lys- and;
Acetyl-Lyss-Thr-Phe-Lys- and;
Acetyl-Lys-Thr-Trp-Lys- and;
H-glutamyl-glycyl-L-arg- and;
pyro-glutamyl-glycyl-L-arg- and;
H-D-isoleucyl-L-prolyl-L-arg-;
Pro-Phe-Arg- and;
Ala-Arg-ArG-;
His-Ser-Ser-Lys-Leu-Gln- and;
N-Glutaryl-(4-hydroxypropyl)Ala-Ser-Cyclohexaglycyl-Gln-Ser-Leu-;
Boc-Gln-Ala-Arg- and;
Boc-benzyl-Glu-Gly-Arg- and;
Boc-Leu-Gly-Arg- and;
Boc-benzyl-Asp-Pro-Arg- and;
Boc-Phe-Ser-Arg- and;
Boc-Val-Pro-Arg- and;
succinyl-Ala-Phe-Lyss- and;
Boc-Leu-Arg-Arg-; and;
Boc-Gly-Lys-Arg-and; and
Boc-Leu-Ser-Thr-Arg-;

Wherein the C terminal carboxyl group is the site of attachment.

In preferred embodiments of the above T is selected from the following structures: 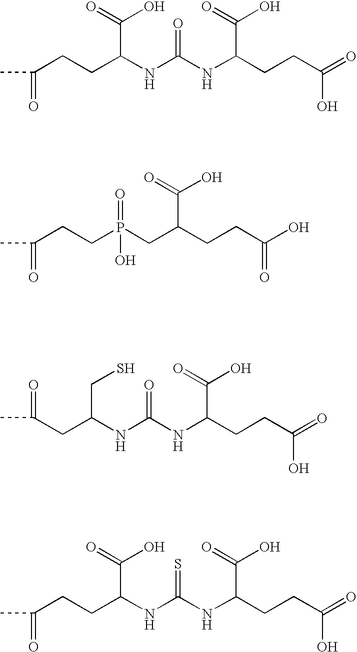
wherein the dashed line is the site of linker attachment.

A preferred embodiment Compound 2 for use in conjunction with the above Compound 1 has the following structure: 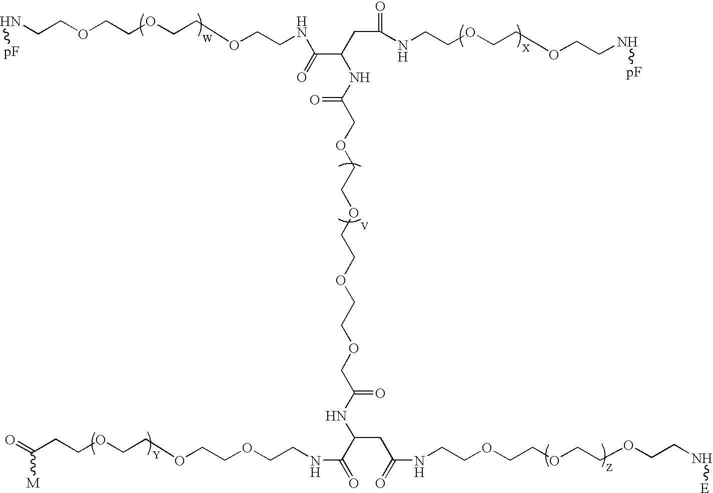
where v=0, 1, 2, 3, 4, 5, 6, . . . 150 or about 150;
where w=0, 1, 2, 3, 4, 5, 6, . . . 150 or about 150;
where x=0, 1, 2, 3, 4, 5, 6, . . . 150 or about 150;
where y=0, 1, 2, 3, 4, 5, 6, . . . 150 or about 150;
where z=0, 1, 2, 3, 4, 5, 6, . . . 150 or about 150;
and wherein the wavy lines are the sites of attachment of the linker to other components indicated; and wherein pF have the following structures: 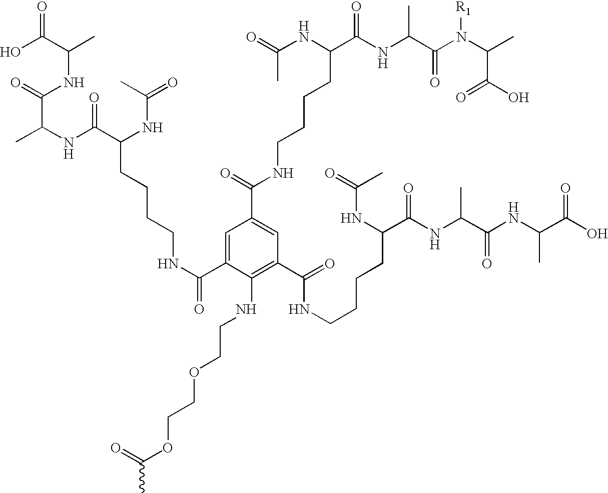
wherein R1 is a bioreversible protecting group; and wherein the wavy line is the site of linker attachment; and wherein the group M is a trimer of vancomycin with the following structure: wherein the wavy line is the site of linker attachment: 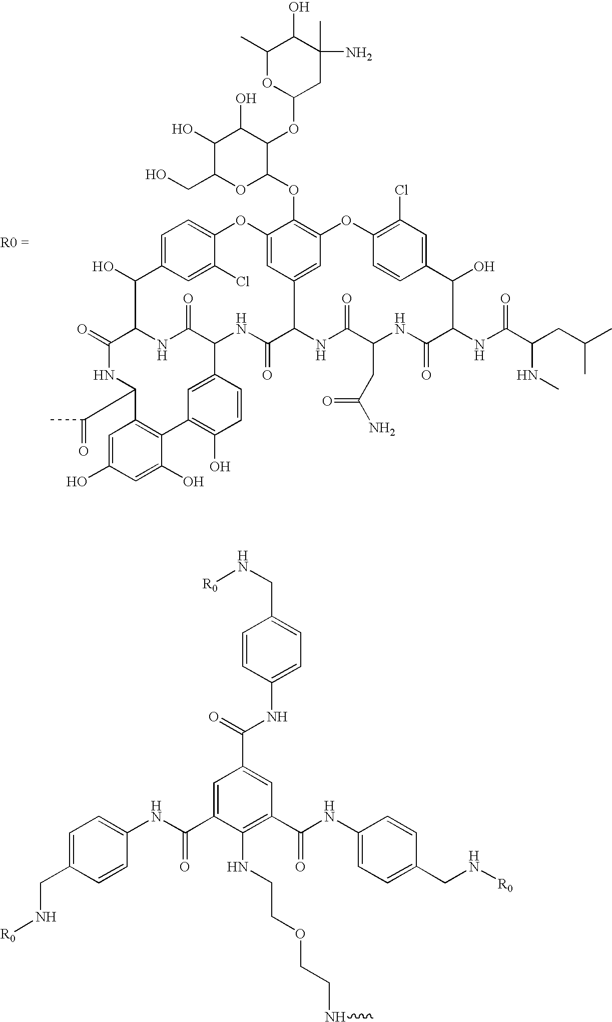
wherein the wavy line is the site of linker attachment; and wherein E is an effector agent.

In preferred embodiments of the above:

v=w=x=y=z=1,2,3,4,5,6,7,8,9,10,11,12,13,14,15,16,17,18,19 20 or about 20;

In a preferred embodiment of the above v=w=x=y=z=10; and R1 has the following structure:

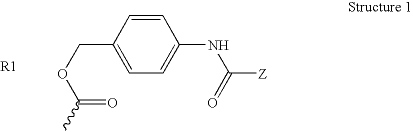

and wherein Z-C(O)— are selected from the following structures that are preferentially cleaved by plasmin:

D-Val-Leu-Lys-
and;
Acetyl-Lys-Thr-Tyr-Lys-
and;
Acetyl-Lys-Thr-Phe-Lys-
and;
Acetyl-Lys-Thr-Trp-Lys-
and;

wherein the carboxy group of the lysine residue is the site of attachment;

and the following structures that are preferentially cleaved by urokinase:

H-glutamyl-glycyl-L-arg- and;
pyro-glutamyl-glycyl-L-arg- and;
H-D-isoleucyl-L-prolyl-L-arg-;

wherein the carboxy group of the arginine is the site of attachment;

and the following structure which is cleaved by human glandular kallikrein 2:

Pro-Phe-Arg-
and;
Ala-Arg-Arg-;

wherein the carboxy group of the arginine is the site of attachment;

and the following structure which is cleaved by PSA:

His-Ser-Ser-Lys-Leu-Gln- and;
N-Glutaryl-(4-hydroxypropyl)Ala-Ser-Cyclohexyaglycyl-Gln-Ser-Leu-;

Wherein the site of attachment is at the carboxy group of the GLn and the Leu respectively;
and E is a cytotoxic drug connected directly to the linker or indirectly by a trigger. In a preferred embodiment of the above E has the following structure: 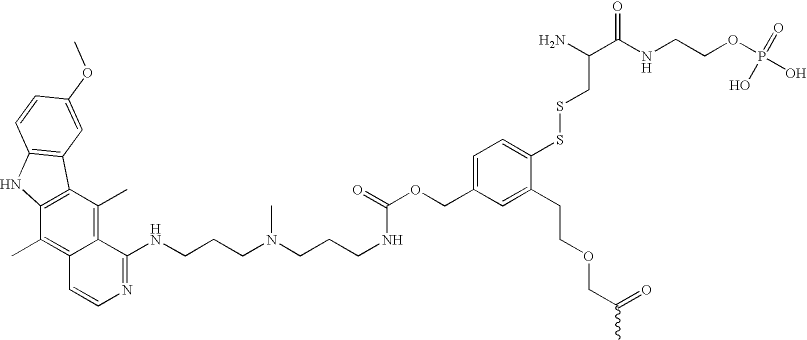
wherein the wavy line is the site of linker attachment.

### SOME PREFERRED EMBODIMENTS BASED ON PEPTIDE NUCLEOTIDE ANALOGS

In a preferred embodiment of the present invention Compound 1 has the following structure:

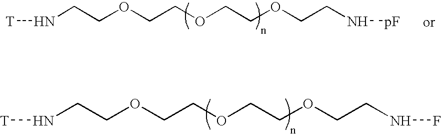

Wherein T is a targeting agent; n=0,1,2,3,4,5,6,7,8,9,10, . . . 200 or about 200; and F is a female adaptor that can bind to a male ligand designated as “M”; and pF is a masked female adaptor that when unmasked yields the group F that can bind to M; and wherein T and F are attached by amide or urea linkages.

In a preferred embodiment Compound 1 has the following structure: 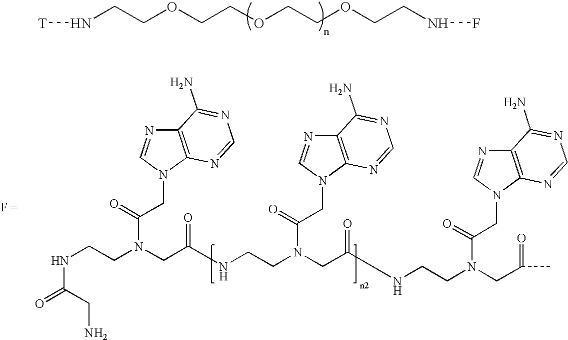
wherein n2=5,6,7,8,9,10,11,12,13,14,15,16,17,18,19, or 20 or about 20. and wherein T is a targeting agent that binds to the target.

In a preferred embodiment of the above the target agent binds to PSMA. In a preferred embodiment T has one of the following structures: 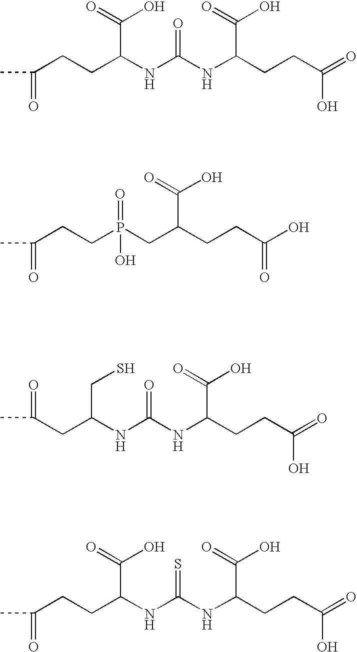
wherein the dotted lines are the sites of attachment to amino groups.

In preferred embodiments the targeting ligand T can bind to MMP1, 2, 3, 9 or MT-MMP-1 and the following structures: 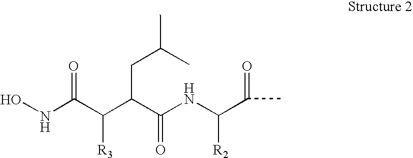
wherein R₂ is benzyl and R₃ is 2-thienylthiomethyl; or wherein R₂ is 5, 6, 7, 8, -terahydro-1-napthyl)methyl and R₃ is methyl; or wherein R₂ is t-butyl and R₃ is OH; or wherein R₂ is H and R₃ is (indol-3-yl)methyl; and wherein the dotted line is the site of linker attachment.

In another preferred embodiment of the above the targeting ligand T can bind to a tumor associated antigen and the group T is a monoclonal antibody. Methods of coupling amino bearing compounds to monoclonal antibodies are well known to one skilled in the arts.

In a preferred embodiment Compound 1 has the structure:
T-L-F or T-L-pF
and Compound 2 has the structure: 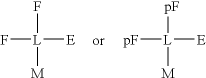
wherein L is a linker; M is a male ligand that can bind to the female adaptor F, pF is a masked female adaptor which when unmasked is converted into F; E is an effector agent; and T is a targeting ligand.

In a preferred embodiment Compound 1 has the following structure: 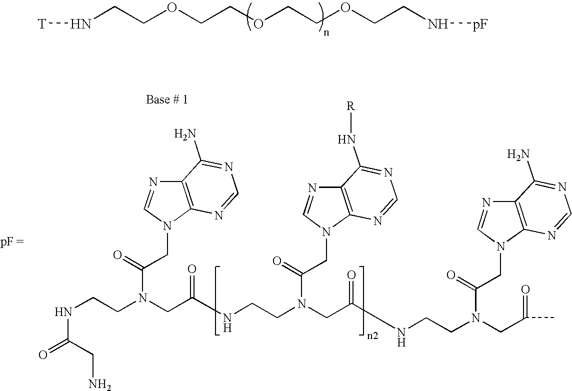
wherein n2=5,6,7,8,9,10,11,12,13,14,15,16,17,18,19, or 20 or about 20. and wherein T is a targeting agent that binds to the target; and wherein R is H, or a bioreversible protecting group; and wherein at least one of the n2 bases has a group R that is not H. In a preferred embodiment n2=14. In a preferred embodiment only one base has a group R that is not H. In a preferred embodiments the subsituted base in which R is not hydrogen is in position number 2,3,4,5,6,7,8,9,10,11,12,13,14,15,16,17,18,19, or 20 where base number 1 is the adenine at the glycine substituted terminus of the oligonucleotide analog. In a preferred embodiment n2=14, and the substituted base is in position number 8. In a preferred embodiment of the above R is the previously designated Structure 1.

In a preferred embodiment of the above the target agent T can bind to PSMA. In a preferred embodiment T has one of the following structures: 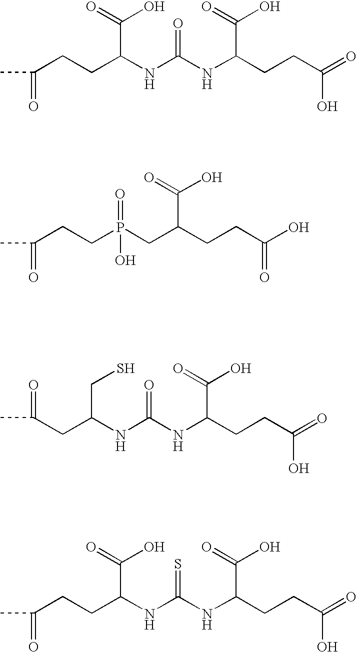
wherein the dotted lines are the sites of attachment to amino groups.

In preferred embodiments the targeting ligand T is the previously designated Structure 2.

In another preferred embodiment of the above the targeting ligand T can bind to a tumor associated antigen and the group T is a monoclonal antibody.

In a preferred embodiment Compound 1 has the following structure: 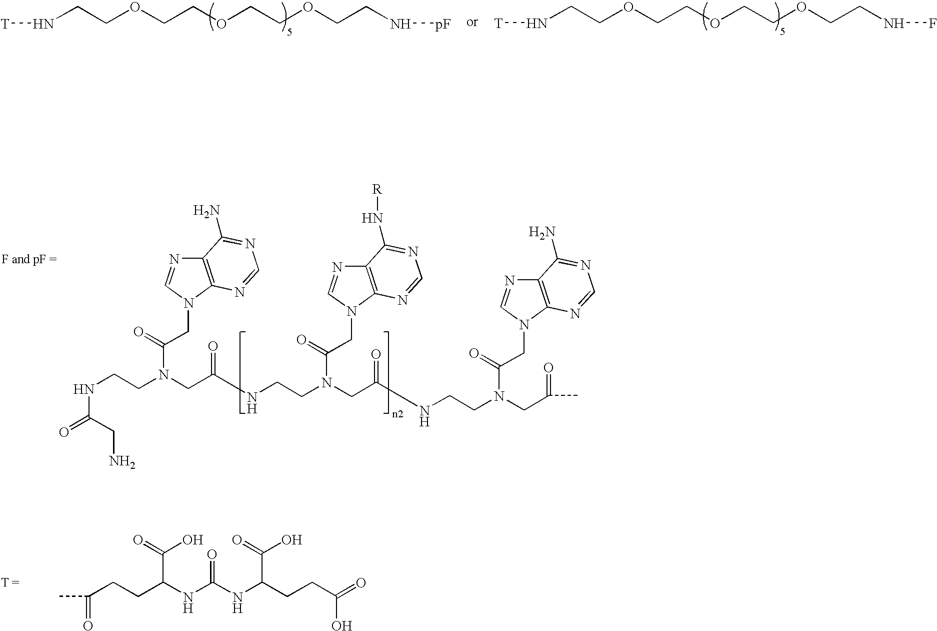
wherein R is H in the group F and wherein R has the previously described Structure 2 in group pF:

In a preferred embodiment of the invention Compound 2 has the following structure: 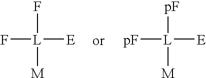
wherein L is a linker; M is a male ligand that can bind to the female adaptor F, pF is a masked female adaptor which when unmasked is converted into F; and E is an effector agent.

In a preferred embodiment of Compound 2 has the following structure: 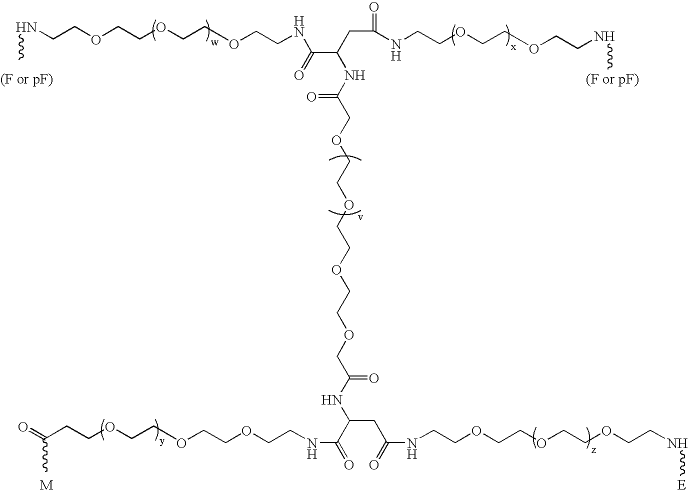
where V=0, 1, 2, 3, 4, 5, 6, . . . 150 or about 150;
where w=0, 1, 2, 3, 4, 5, 6, . . . 150 or about 150;
where x=0, 1, 2, 3, 4, 5, 6, . . . 150 or about 150;
where y=0, 1, 2, 3, 4, 5, 6, . . . 150 or about 150;
where z=0, 1, 2, 3, 4, 5, 6, . . . 150 or about 150;
and wherein the wavy lines are the sites of attachment of the linker to other components indicated; and wherein F and pF have the following structures: 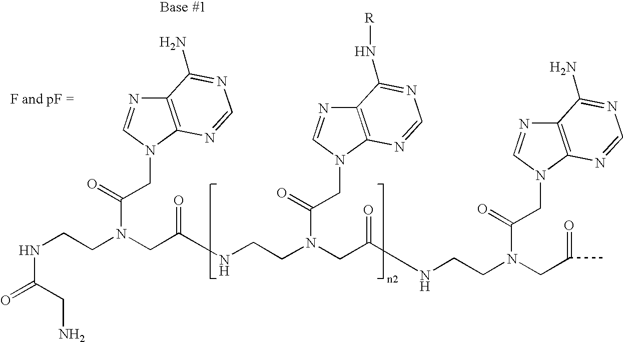
wherein n2=5,6,7,8,9,10,11,12,13,14,15,16,17,18,19, or 20 or about 20; and wherein R is H, or a bioreversible protecting group; and wherein for the group pF at least one of the n2 bases has a group R that is not H; and wherein R is H in the group F; and wherein the dotted line is the site of linker attachment; and wherein the group M has the following structure: 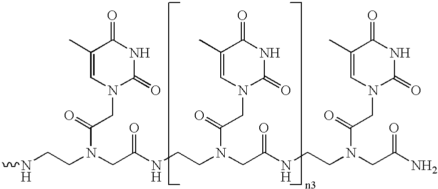
wherein n3=5,6,7,8,9,10,11,12,13,14,15,16,17,18,19, or 20 or about 20; wherein the way line is the site of linker attachment; and wherein E is an effector agent.

In preferred embodiments of the above:

v=w=x=y=z=1,2,3,4,5,6,7,8,9,10,11,12,13,14,15,16,17,18,19 20 or about 20;

n2=n3=14;

R is H; except for the R on the base of position number 8; where base number 1 is the adenine at the glycine substituted terminus of the oligonucleotide analog;

wherein R has the previously given Structure 2.

In preferred embodiments of the above: v=w=x=y=z=10;
and E is a cytotoxic drug connected directly to the linker or indirectly by a trigger. Some preferred embodiments of the above E are shown below wherein the wavy line is the site of attachment:

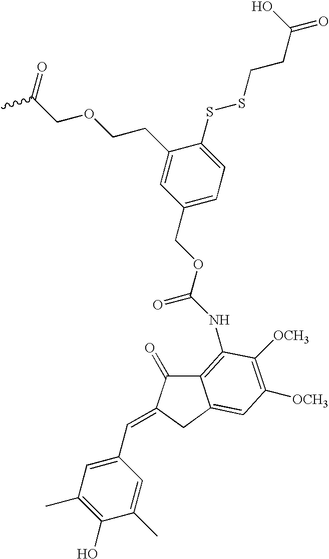

In this case the drug indanocine can be released intracellularly upon reduction of the disulfide bond. The following reference relates to this matter: Leioni L., et al., “Indanocine, a Microtubule-Binding Indanone and a Selective Inducer of Apoptosis in Multidrug-Resistant Cancer Cells,” J Nat Cancer Inst, 92(3):217-224 (2000) the contents of which are incorporated herein by reference in their entirety.

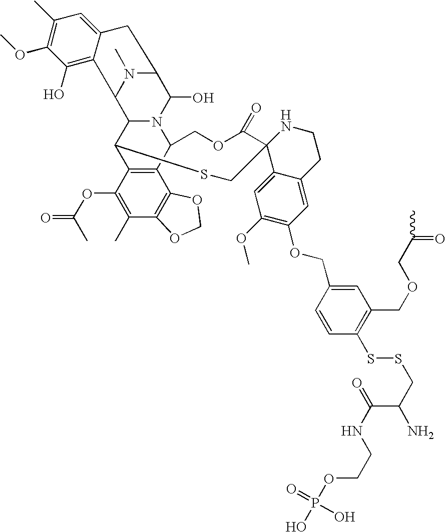

In this embodiment the drug Ecteinascidin 743 will be liberated following activation of the intracellular trigger by intracellular glutathione or by thioreductases. Ecteinascidin 743 is cytotoxic at picomolar concentrations. The following references relate to this subject matter: Zewail-Foote M.; Hurley L. H., “Ecteinascidin 743: A Minor Groove Alkylator that Bends DNA toward the Major Groove,” J Med Chem, 42(14):2493-2497 (1999); Takebayashi Y., et al., “Poisoning of Human DNA Topoisomerase I by Ecteinascidin 743, an Anticancer Drug that Selectively Alkylates DNA in the Minor Groove,” Proc Natl Acad Sci USA, 96:7196-7201 (1999); Hendriks H. R., et al., “High Antitumour Activity of ET743 against Human Tumour Xenografts from Melanoma, Non-Small-Cell Lung and Ovarian Cancer.” Ann Oncol, 10(10):1233-40 (1999), the contents of which are incorporated herein by reference in their entirety.

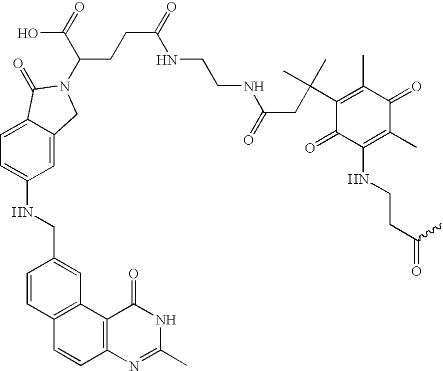

In this preferred embodiment The N-(2-Amino-ethyl)-amide derivative of the toxin BW1843U89 will be liberated following activation of the intracellular trigger by quinone reductase. BW1843U89 inhibits thymidylate synthase at picomolar concentrations. X-ray crystallography of BW1843U89 bound to ecoli thymidylate synthase reveals the carboxylate groups to be free and solvent exposed. Accordingly, the presence of the amino-ethyl group should not impair binding to the thymidylate. synthase. The following reference relates to this subject matter: Stout, T. J.; Stroud, R. M., “The Molecular Basis of the Anti-Cancer Therapeutic, BW1843U89, with Thymidylate Synthase at 2.0 Angstroms Resolution,” Protein Data Bank (1996) File 1SYN, the contents of which are incorporated herein by reference in their entirety.

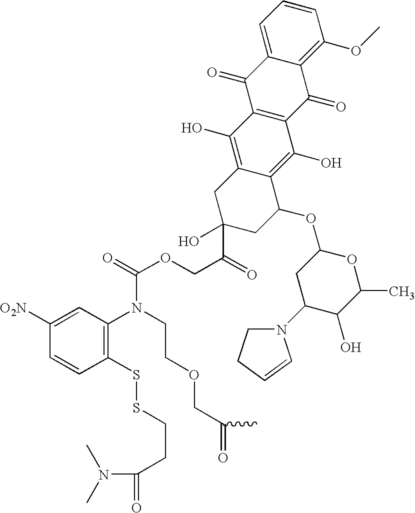

In this preferred embodiment the highly potent toxin 2-pyrrolinodoxorubicin will be liberated upon activation of an intracellular disulfide trigger. Cleavage of the disulfide by thiol reductases will unmask a thiol group, which will, via an intramolecular nucleophilic reaction, cleave the carbamate group and release the toxin. The following references relate to this subject matter: Nagy A., et al., “High Yield Conversion of Doxorubicin to 2-pyrrolinodoxorubicin, an Analog 500-1000 Times More Potent: Structure-Activity Relationship of Daunosamine-Modified Derivatives of Doxorubicin,” Proc Natl Acad Sci USA, 93:2464-2469; the contents of which are incorporated herein by reference in their entirety.

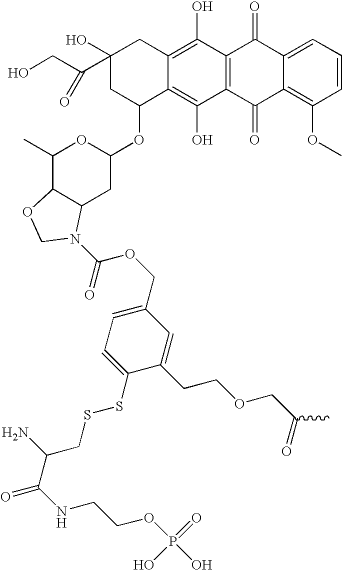

In this embodiment doxorubicin mono-oxazolidine will be released upon reduction of the disulfide bond. Formaldehyde conjugates of doxorubicin are approximately 50-150 times more potent than doxorubicin and up to 10,000 fold more potent than doxorubicin in adriamycin resistant MCF-7/ADR cells. The following references relate to this subject matter: Taatjes D .J., et al., “Epidoxoform: A Hydrolytically More Stable Anthracycline-Formaldehyde Conjugate Toxic to Resistant Tumor Cells”, J Med Chem, 41:1306-1314 (1998).; Fenick D. J., et al., “Doxoform and Daunoform: Anthracycline-Formaldehyde Conjugates Toxic to Resistant Rumor Cells”, J Med Chem, 40:2452-2461 (1997).; the contents of which are incorporated herein by reference in their entirety.

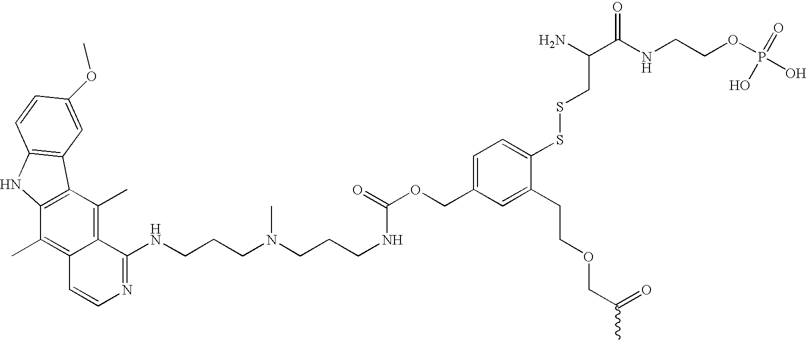

In this embodiment a highly cytotoxic ellipticine analog will be released after activation of an intracellular trigger by thioreductase. The following references relate to this subject matter: Bisagni E., et al., “Synthesis of 1-Substituted Ellipticines by a New Route to Pyrido[4,3-b]-carbazoles,” JCS Perkin I, 1706-1711 (1978); Czerwinski G., et al., “Cytotoxic Agents Directed to Peptide Hormone Receptors: Defining the Requirements for a Successful Drug,” Proc Natl Acad Sci USA, 95:11520-11525 (1998), the contents of which are incorporated herein by reference in their entirety.

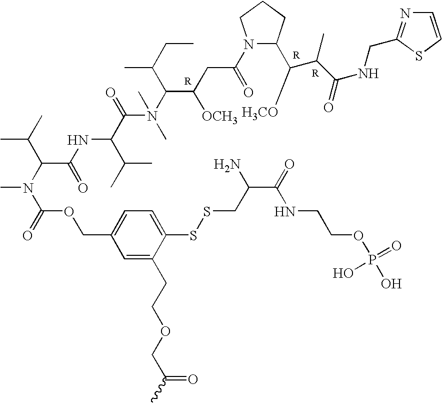

In this embodiment a highly cytotoxic dolastatin 10 analog will be released upon disulfide reduction. The following references relate to this subject matter: U.S. Pat. No. 6,004,934 Dec. 21, 1999 Sakakibara et al., “Tetrapeptide Derivative”; the contents of which are incorporated herein by reference in their entirety.

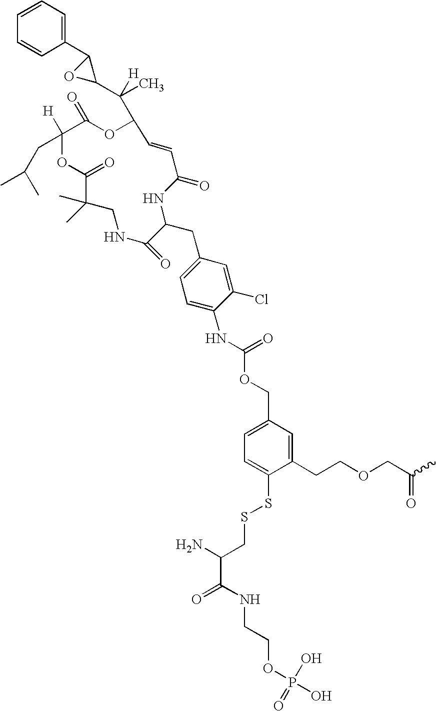

In this embodiment a derivative of cryptophycin that is toxic at picomolar concentrations will be freed upon cleavage of a disulfide trigger by thiol reductases. The following references relate to this subject matter: Showell G. A., et al., “High-Affinity and Potent, Water-Soluble 5-Amino-1,4-Benzodiazepine CCKB/Gastrin Receptor Antagonists Containing a Cationic Solubilizing Group,” J Med Chem, 37(6):719-21 (1994); Panda D., et al., “Antiproliferative Mechanism of Action of Cryptophycin-52: Kinetic Stabilization of Microtubule Dynamics by High-Affinity Binding to Microtubule Ends,” Proc Natl Acad Sci USA, 95:9313-9318 (1998); Smith C. D., et al., “Cryptophycin: A New Antimicrotubule Agent Active against Drug-resistant Cells,” Cancer Res, 54:3779-3784 (1994); Patel V. F., et al., “Novel Cryptophycin Antitumor Agents: Synthesis and Cytotoxicity of Fragment “B” Analogues,” J Med Chem, 42:2588-2603 (1999), the contents of which are incorporated herein by reference in their entirety.

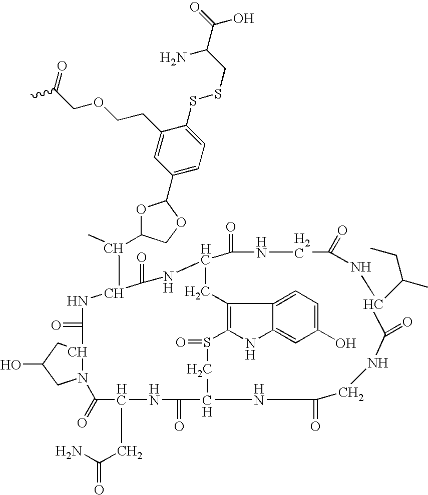

In this embodiment α Amanitin will be liberated upon disulfide reduction. α Amanitin is a potent cytoxic agent that inhibits RNA polymerase II. α Amanitin triggers degradation of a subunit of RNA polymerase II and inhibits denovo synthesis of RNA polymerase thereby setting off an irreversible chain of events that culminate in cell death. α Amanitin has been used in the past as a toxin in complex with monoclonal antibodies. α Amanitin is cytotoxic for nonproliferating cells. This is a potential advantage for the treatment of cancers that have a low mitotic index. The following references relate to this subject matter: Nguyen V T, Giannoni F, Dubois M F, Seo S J, Vigneron M, Kedinger C, Bensaude O.; “In vivo degradation of RNA polymerase II largest subunit triggered by alpha-amanitin”. Nucleic Acids Res 1996 ;24(15):2924-9; Koumenis C, Giaccia A, “Transformed cells require continuous activity of RNA polymerase II to resist oncogene-induced apoptosis.” Mol Cell Biol 1997 (12):7306-16; and Davis M T, Preston J F . “A conjugate of alpha-amanitin and monoclonal immunoglobulin G to Thy 1.2 antigen is selectively toxic to T lymphoma cells.” Science 1981;213(4514):1385-8; the contents of which are incorporated herein by reference in their entirety.

In a preferred embodiment of the above E is a chelating group with a bound radionuclide. A large number of suitable chelating groups and radionuclides of therapeutic and diagnostic utility are well known to one skilled in the art. The following reference is related to this matter: Shuang Liu ; D. Scott Edwards “Bifunctional Chelators for Therapeutic Lanthanide Radiopharmaceuticals “Bioconjugate Chem., 12 (1), 7-34, 2001; the contents of which are incorporated herein by reference in their entirety.

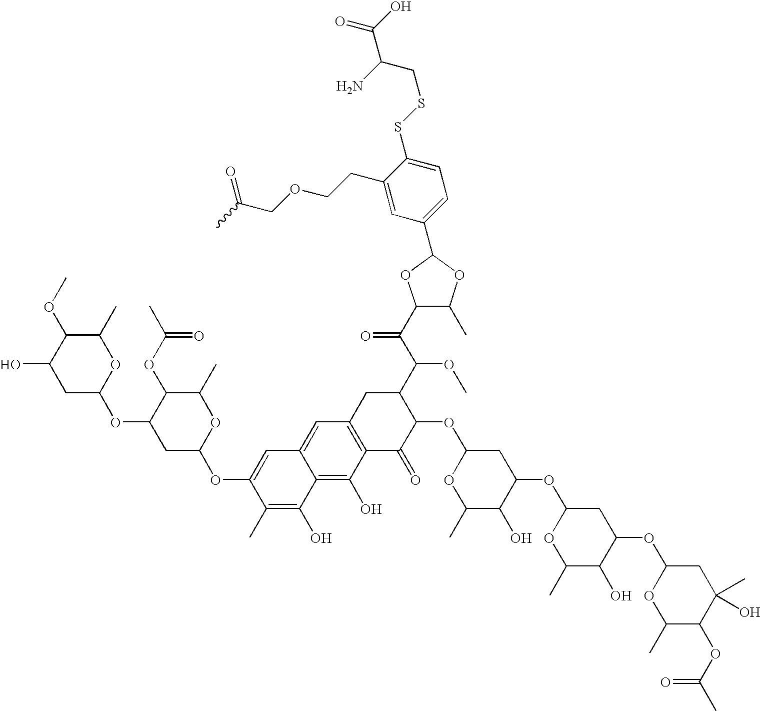

In this embodiment Chromomycin A3 will be released upon disulfide reduction. Chromomycin A3 is cytotoxic to cells including adriamycin resistant tumor lines at subnanomolar concentrations. The drug binds strongly to DNA and inhibits RNA synthesis.

The present invention also includes a compound; wherein said compound is a prodrug that can undergo biotransformation into a drug; wherein said drug gains the ability to selectively bind at least one additional molecule of the prodrug; and wherein bound prodrug can undergo biotransformation into the drug which can selectively bind additional molecules of the prodrug.

A preferred embodiment of the above is a compound that can undergo biotransformation into a drug; wherein said drug can bind at least two molecules of the prodrug.

A preferred embodiment of the above is a compound comprised of at least one male ligand; at least one masked female adaptor; and at least one effector group; and wherein the masked female adaptors cannot bind to the male ligands; and wherein the masked female adaptors can be unmasked by the action of a triggering enzyme or other biomolecules to yield female adaptors; and wherein each female adaptor can bind to at least one male ligand; and each male adaptor can bind to at least one female adaptor; and wherein the effector group is a group that directly or indirectly exerts an activity at the target.

A preferred embodiment of the above is a compound comprised of:
{[M]ₘ and [E]ₒ and [PF]ₙ}
wherein M is a male ligand; E is an effector group; and wherein the groups M can be the same or different; and wherein the groups E can be the same or different; and wherein the groups pF can be the same or different; and wherein o is an integer between 1 and about 10; and m is an integer between 1 and about 200; and n is an integer between 1 and about 200.

A preferred embodiment of the above is a compound with the following structure: 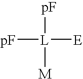
and wherein L is a linker.

A preferred embodiment of the above is a compound wherein M is an oligonucleotide or oligonucleotide analog in which the number of bases is between about 10 to about 25.

A preferred embodiment of the above is a compound wherein M is an oligo-peptide nucleotide analog and pF is a masked oligo-peptide nucleotide analog.

A preferred embodiment of the above is a compound in which M has the structure: 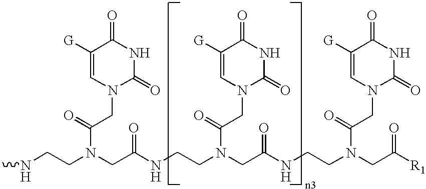
wherein the wavy line is the site of linker attachment; G is H, or methyl; and
wherein R₁ is OH; NH2; NH—CH2-CH2-CH2-P(O)(OH)2; or NH—R2; wherein NH2R2 is an amino acid, or wherein R1 is an inert group; and where n3 is an integer between 8 and 23.

A preferred embodiment of the above is a compound wherein M has the structure:

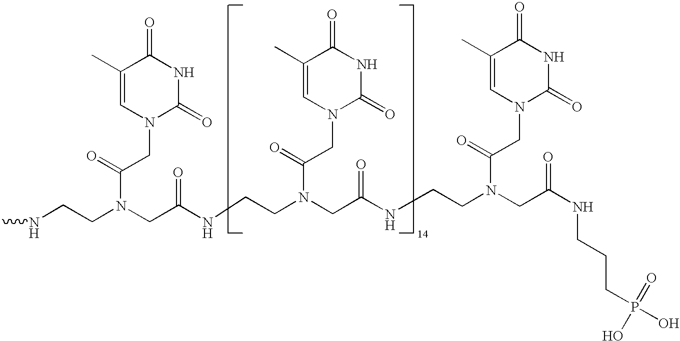

A preferred embodiment of the above is a compound wherein pF has the structure: 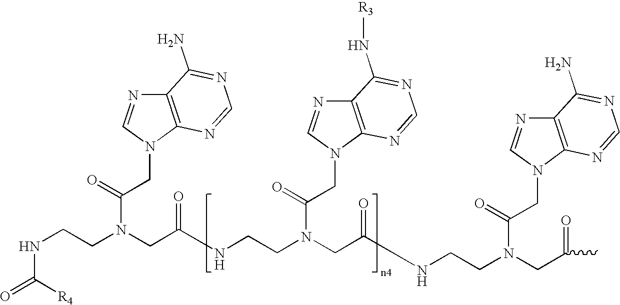
wherein the wavy line is the site of linker attachment; and n4 is an integer between 8 and about 25; and R3 is H or a masking group that can be removed by the triggering enzyme; wherein at least one of the groups R3 is a masking group; and wherein R4C(O)OH is glycine, lysine, —CH2-CH2-CH2-P(O)(OH)2; or an inert group.

A preferred embodiment of the above is a compound wherein pF has the structure: 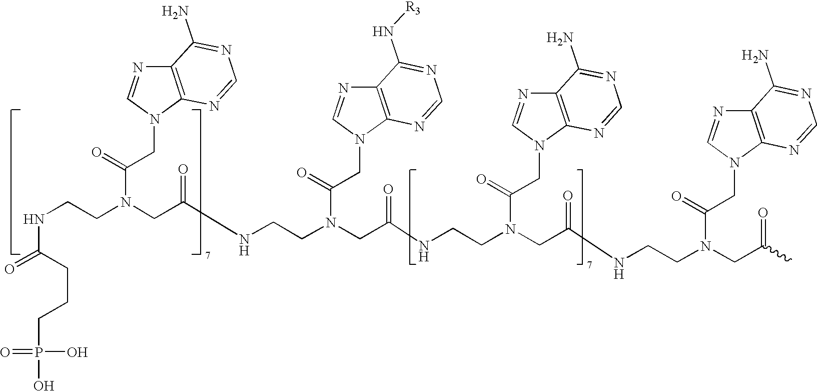
and wherein R3 has the structure: 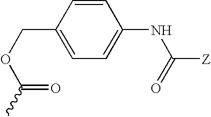
wherein the wavy line is the site of attachment; and wherein Z is selected such that the triggering enzyme can cleave the corresponding amide.

A preferred embodiment of the above is a compound wherein Z-C(O)OH is an amino acid, or an oligo-peptide comprised of between 2 and about 25 amino acids; or analogs thereof.

A preferred embodiment of the above is a compound wherein Z-C(O)— are selected from the following groups:

D-Val-Leu-Lys-
and;
Acetyl-Lys-Thr-Tyr-Lys-
and;
Acetyl-Lys-Thr-Phe-Lys-
and;
Acetyl-Lys-Thr-Trp-Lys-
and;
H-glutamyl-glycyl-L-arg-
and;
pyro-glutamyl-glycyl-L-arg-
and;
H-D-isoleucyl-L-prolyl-L-arg
Pro-Phe-Arg-
and;
Ala-Arg-Arg-;
His-Ser-Ser-Lys-Leu-Gln-
and;
N-Glutaryl-(4-hydroxypropyl)Ala-Ser-
Cyclohexaglycyl-Gln-Ser-Leu-;

A preferred embodiment of the above is a compound with the following structure: 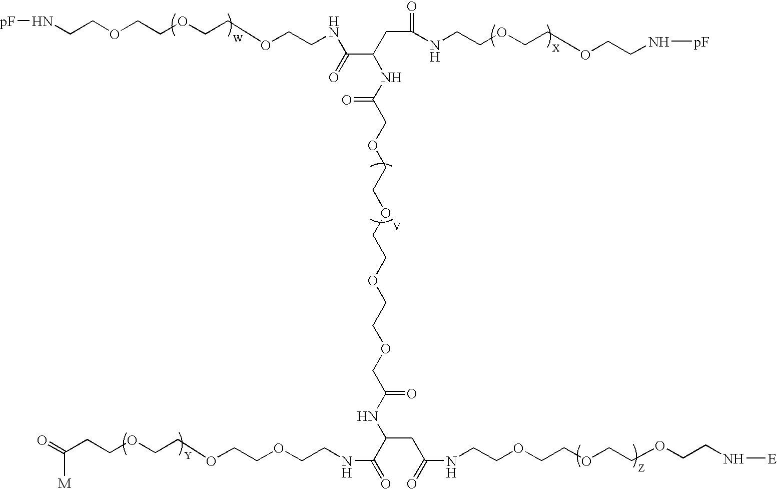
and wherein v,w,x,y, and z are independent integers between 0 and about 150.

A preferred embodiment of the above is a compound wherein E is selected from the following structures: 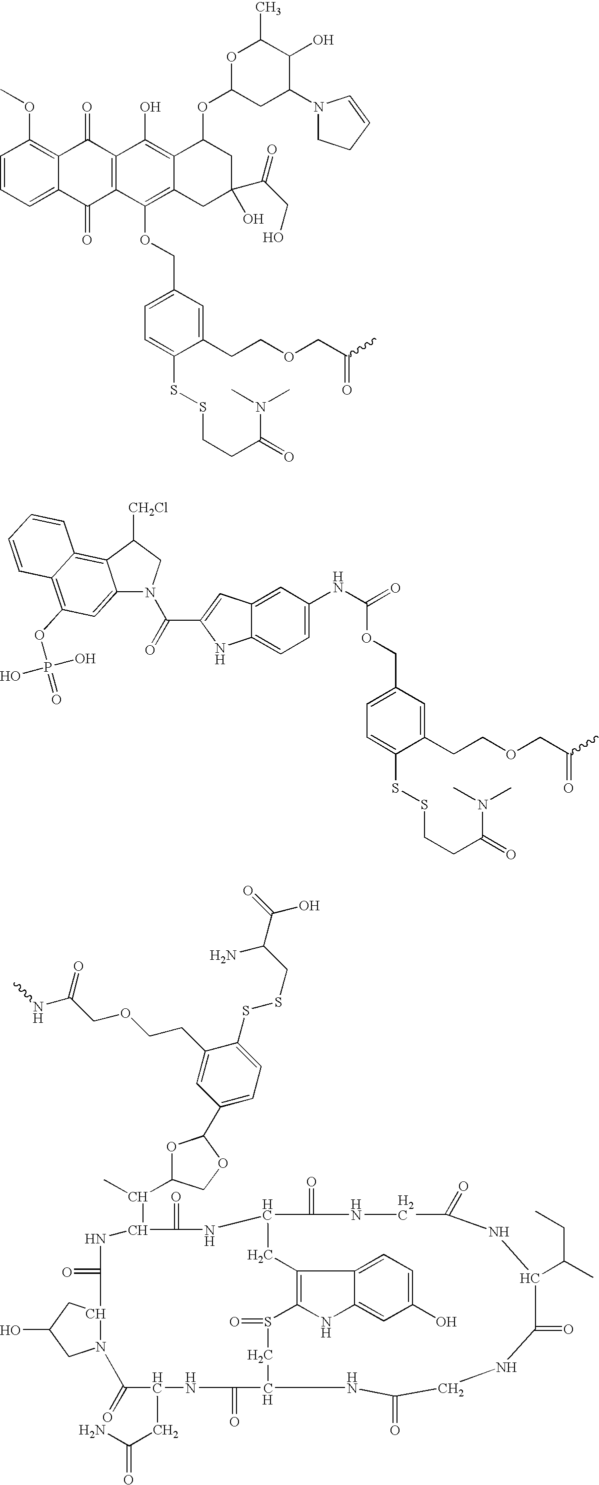 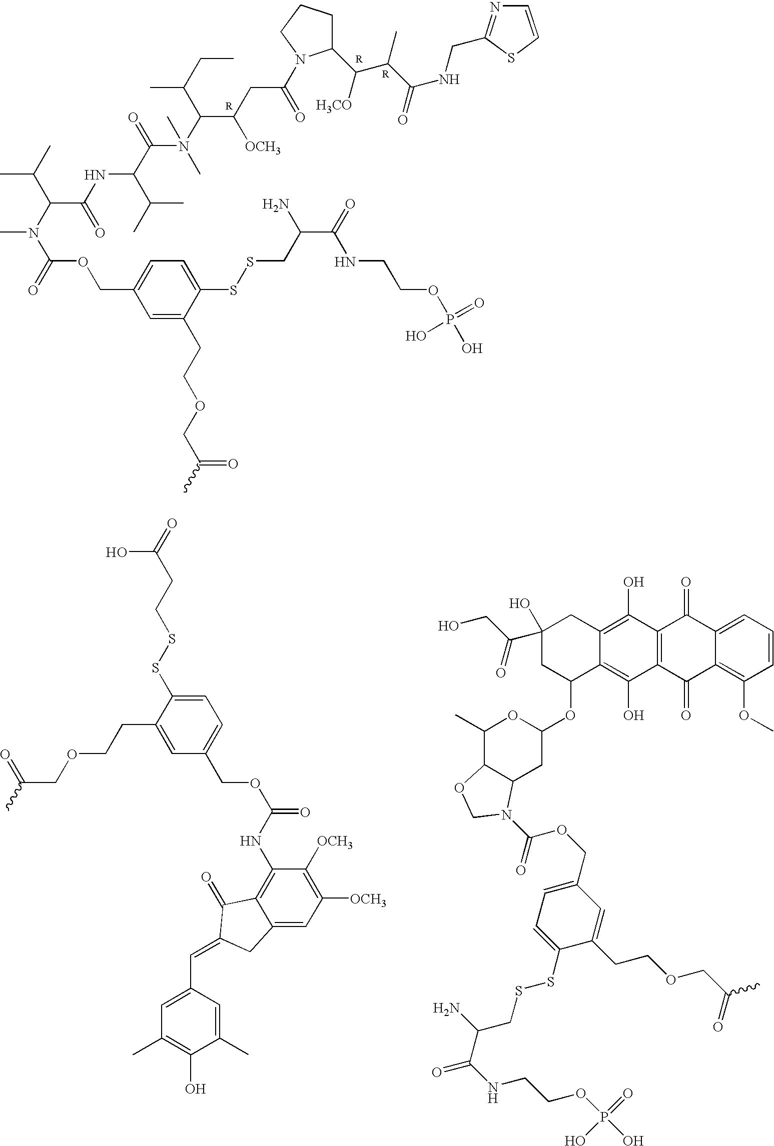 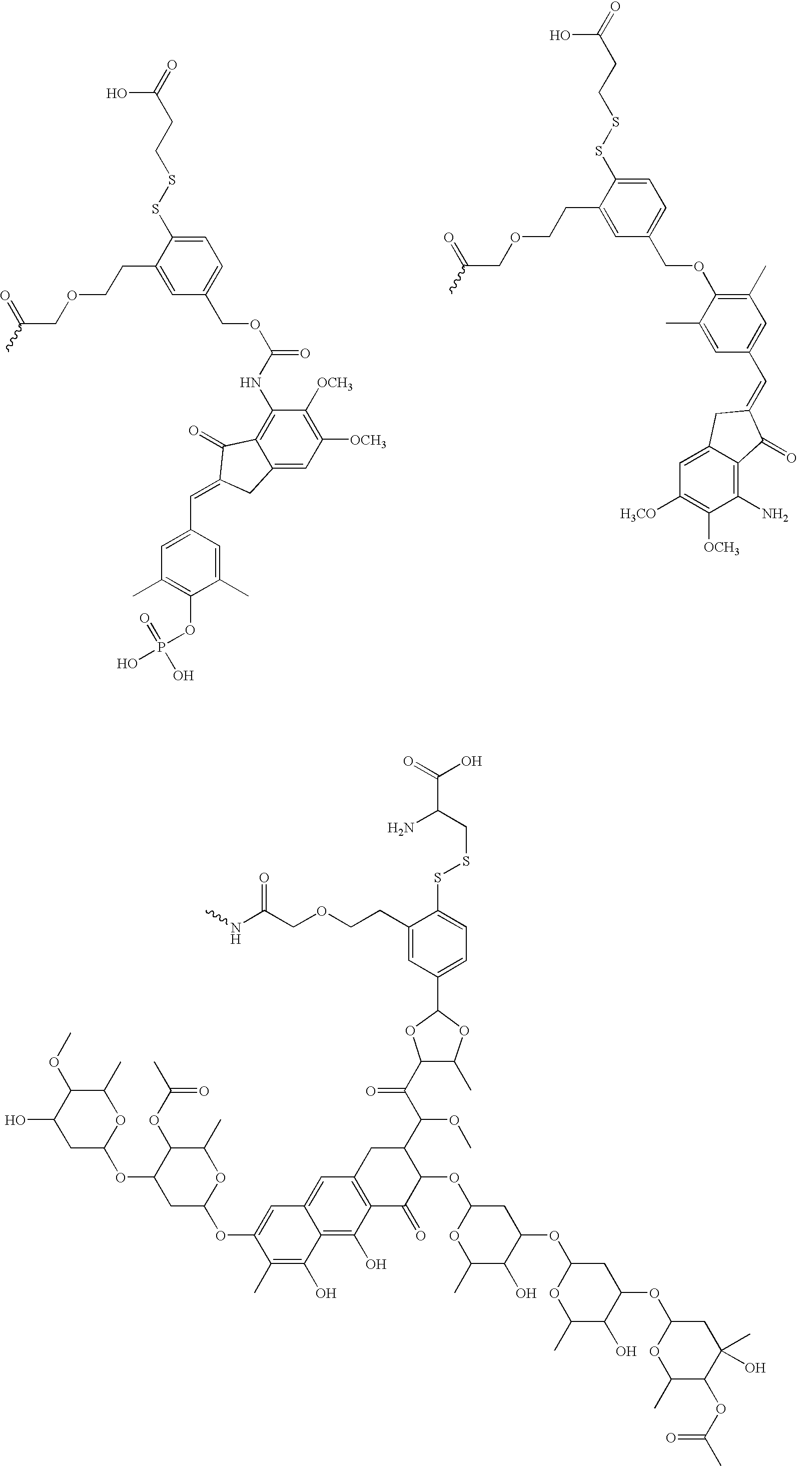
wherein the way line is the site of linker attachment.

A preferred embodiment of the above is a compound wherein v=10; w=10; x=10; y=10 and z=10.

The present invention also includes a prodrug that can undergo biotransformation into a drug wherein said drug gains the ability to selectively bind to at least one molecule of a second type of drug compound.

A preferred embodiment of the above is a prodrug that is comprised of a targeting agent that can bind to a target receptor; and at least one masked female adaptors; wherein the masked female adaptors cannot bind to the male ligands; and wherein the masked female adaptors can be unmasked by the action of a triggering enzyme to yield female adaptors; and wherein each female adaptor can bind to at least one male ligand; and each male adaptor can bind to at least one female adaptor; and wherein the male adaptors are groups present on the second type of drug compound.

A preferred embodiment of the above is a compound comprised of the groups:
{T and [pF]_{q}}
wherein T is a targeting agent that can bind to R; wherein R is a receptor at the target; and wherein each pF is independently a masked female adaptor; and wherein q is an integer between 1 and about 200; and wherein the groups pF can be the same or different.

A preferred embodiment of the above is a compound wherein T is tumor selective.

A preferred embodiment of the above is a compound wherein T can bind to a receptor selected from the following group: Prostate Specific Membrane Antigen; Somatostatin receptors; Luteinizing releasing hormone receptor; Bombesin/gastrin releasing peptide receptor; Sigma receptor; STEAP antigen; Prostate Stem Cell Antigeri; Platelet Derived Growth Factor alpha receptor; Hepsin; PATE; Gonadotropin-Releasing Hormone receptor; Transmembrane serine protease (TMPRSS2); tissue factor; c-Met; Urokinase; Urokinase receptor; MMP-1, MMP-2, MMP-7, MMP-9; and MMP-14.
A preferred embodiment of the above is a compound with the structure: 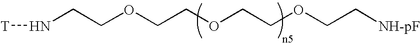
wherein n5 is an integer between 0 and about 200.

A preferred embodiment of the above is a compound wherein pF is a masked oligonucleotide or masked oligonucleotide analog in which the number of bases is between about 10 to about 25.

A preferred embodiment of the above is a compound wherein pF a masked oligo-peptide nucleotide analog.

A preferred embodiment of the above is a compound wherein pF has the structure: 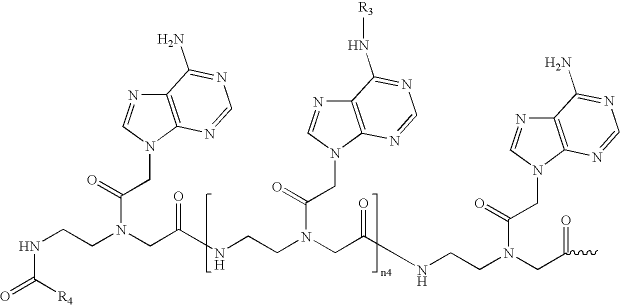
wherein the wavy line is the site of linker attachment; and n4 is an integer between 8 and about 25; and R3 is H or a masking group that can be removed by the triggering enzyme; wherein at least one of the groups R3 is a masking group; and wherein R4C(O)OH is glycine, lysine, —CH2-CH2-CH2-P(O)(OH)2; or an inert group.

A preferred embodiment of the above is a compound wherein pF has the structure: 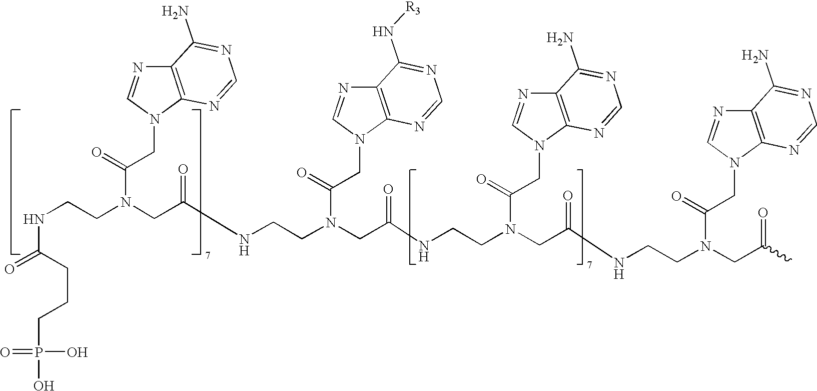
and wherein R3 has the structure: 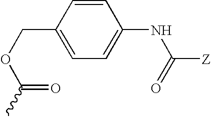
wherein the wavy line is the site of attachment; and wherein Z is selected such that the triggering enzyme can cleave the corresponding amide.

A preferred embodiment of the above is a compound wherein Z-C(O)OH is an amino acid, or an oligo-peptide comprised of between 2 and about 25 amino acids; or an analog thereof.

A preferred embodiment of the above is a compound wherein Z-C(O)— are selected from the following groups:

D-Val-Leu-Lys-
and;
Acetyl-Lys-Thr-Tyr-Lys-
and;
Acetyl-Lys-Thr-Phe-Lys-
and;
Acetyl-Lys-Thr-Trp-Lys-
and;
H-glutamyl-glycyl-L-arg-
and;
pyro-glutamyl-glycyl-L-arg-
and;
H-D-isoleucyl-L-prolyl-L-arg-
Pro-Phe-Arg-
and;
Ala-Arg-Arg-;
His-Ser-Ser-Lys-Leu-Gln-
and;
N-Glutaryl-(4-hydroxypropyl)Ala-Ser-
Cyclohexaglycyl-Gln-Ser-Leu-;

A preferred embodiment of the above is a compound wherein T is selected from the group:

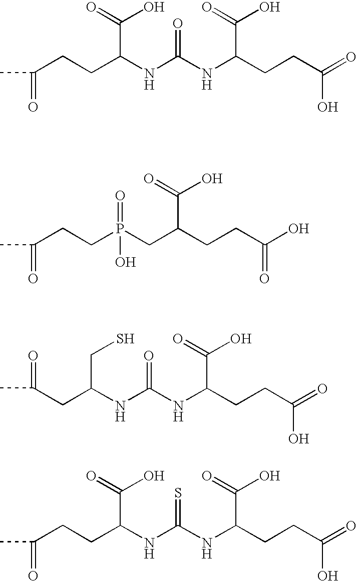

A preferred embodiment of the above is a compound wherein n5 is 10.

Methods of Use

The compounds of the present invention are used by contacting the target cells with a sufficient quantity to evoke the desired diagnostic or therapeutic result. The drugs can be administered in combination with commonly employed pharmacological excipients, preservatives and stabilizers that are well known to one skilled in the arts. The drugs can be administered simultaneously or sequentially. In general, the drugs are for intravenous use and can be administered dissolved in sterile saline or water or a buffered salt solution. In selected situations the drugs could be given routes such as intra-arterially, intra-peritoneally, orally or topically. The scope of the present invention also includes contacting cells in vitro with compounds of the present invention.

The drugs should be administered to a patient or an animal in a sufficient amount and for a sufficient period of time to achieve the desired pharmacological result and will depend upon the severity of the illness and the other factor well known to one skilled in the art. For a drug in which E is comprised of a known drug, the dose of can be lower than or about equal to the dose of drug E as currently used in clinical practice. The dose of the drug administered can be in the range of about 1 picogram per kilogram body weight to about 50 mg/kg.

In a preferred embodiment the drugs are administered at ultra-low dose to achieve nanomolar or sub-nanomolar plasma concentrations. In other embodiments the drug is given at conventional doses similar to those currently used for the drug E. Procedures for dose optimization are well known to one skilled in the art.

The present invention also includes a method to treat a neoplastic disease in an animal or person. The method is comprised of the administration of compounds of the present invention that are targeted to the tumor and wherein said compounds are comprised of an anticancer agent.

For diagnostic use, routine procedures and methodologies applicable to the detection and imaging of the targeted moiety can be used. A preferred embodiment is for tumor imaging said method comprising the administration of a Compound 1 that is targeted to a tumor and a Compound 2 that has an effector group useful for diagnostic imaging.

The present invention also comprises a method for the site specific delivery to a target of effector molecules in vitro or in vivo; wherein said method is comprised of contacting the target with Compound 1 and Compound 2; and wherein Compound 1 is comprised of at least one group that can bind to the target, and at least one masked female adaptor; and wherein Compound 2 is comprised of at least one male ligand; at least one masked female adaptor; and at least one effector group; and wherein the masked female adaptors cannot bind to the male ligands; and wherein the masked female adaptors can be unmasked by the action of an enzyme or other biomolecule at the target site to yield female adaptors; and wherein each female adaptor can bind to at least one male ligand; and each male adaptor can bind to at least one female adaptor; and wherein the effector group is a group that directly or indirectly exerts an activity at the target.

In a preferred embodiment of the above method, Compound 2 is comprised of at least two masked female adaptors.

In a preferred embodiment of the above Compound 1 is comprised of the groups:
{T and [pF]_{q}}
Wherein T is a targeting agent that can bind to R; wherein R is a receptor at the target; and wherein each pF is independently a masked female adaptor; and wherein q is an integer between 1 and about 200; and wherein the groups pF can be the same or different; and wherein Compound 2 is comprised of:
{[M]ₘ and [E]ₒ and [pF]ₙ}
wherein M is a male ligand; E is an effector group; and wherein the groups M can be the same or different; and wherein the groups E can be the same or different; and wherein the groups pF can be the same or different; and wherein o is an integer between 1 and about 10; and m is an integer between 1 and about 200; and n is an integer between 1 and about 200; and wherein the group pF can be unmasked by at least one triggering enzyme at the target.

In a preferred embodiment of the above method q=1; m=1; o=1; and n=2.

In a preferred embodiment of the above method the triggering enzyme is enriched at the target.

In a preferred embodiment of the above method either R, or the triggering enzyme, or both, are enriched at the target compared to at a non-target.

In a preferred embodiment of the above method, Compound 1 has the following structure:
T-L-pF
and Compound 2 has the structure: 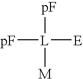
wherein L is a linker.

In a preferred embodiment of the above method the target is a tumor.

In a preferred embodiment of the above method the target is a tumor or both the tumor and the tissue of tumor origin.

In a preferred embodiment of the above method the tumor is prostate cancer.

In a preferred embodiment of the above method T can bind to a receptor R selected from the following group: Prostate Specific Membrane Antigen; Somatostatin receptors; Luteinizing releasing hormone receptor; Bombesin/gastrin releasing peptide receptor; Sigma receptor; STEAP antigen; Prostate Stem Cell Antigen; Platelet Derived Growth Factor alpha receptor; Hepsin; PATE; Gonadotropin-Releasing Hormone receptor; Transmembrane serine protease (TMPRSS2); tissue factor; c-Met; Urokinase; Urokinase receptor; MMP-1, MMP-2, MMP-7, MMP-9; and MMP-14.

In a preferred embodiment of the above method pF can be unmasked by a triggering enzyme selected from the following group: urokinase, plasmin, PSA; hepsin; MMP-1, MMP-2, MMP-7, MMP-9; MMP-14; Transmembrane serine protease; Human glandular kallikrein II; Prostase; and Prostatic acid phosphatase and wherein said triggering enzyme is not R.

In a preferred embodiment of the above method E is a cytotoxic drug or radionuclide bearing group.

Methods of Drug Synthesis

The drugs of the present class can be prepared by a variety of synthetic approaches well known to one skilled in the arts. A modular approach is preferred in which basic components such as linkers, triggers, and ligands are synthesized and coupled. A large variety of methods can be utilized to couple the respective components. Approaches to synthesize the present compounds are similar to those described for the synthesis of multifunctional drug delivery vehicles in Ser. No. 09/712,465 Nov. 15, 2000 Glazier, “Selective Cellular Targeting: Multifunctional Delivery Vehicles, Multifunctional Prodrugs, Use as Neoplastic Drugs. The general steps include chemical protection of interfering groups, coupling, and deprotection. A preferred type of coupling reaction is the formation of an amide or ester bond. General references are given below and synthetic methodologies illustrated by examples that follow. The following references relate to this subject matter: Bodanszky M.; Bodanszky A. (1994) “The Practice of Peptide Synthesis” Springer-Verlag, Berlin Heidelberg; Greene, Theodora W.; Wuts, Peter G. M. (1991) “Protective Groups in Organic Synthesis” John Wiley & Sons, Inc.; March, Jerry (1985) “Advanced Organic Chemistry”, John Wiley & Sons Inc., the contents of which are incorporated herein by reference in their entirety.

The terms “coupled” or “coupling” are used to refer to the formation of an ester or amide bond from an alcohol or amine and acid. A large number of agents and methods are well known to one skilled in the arts for the coupling of amine or alcohols to acids. Relevant coupling agents and methods may be found within the following references :Bodanszky M.; Bodanszky A. (1994) “The Practice of Peptide Synthesis” Springer-Verlag, Berlin Heidelberg; Trost, Barry; (1991) Comprehensive Organic Synthesis, Pergamon Press, the contents of which are incorporated herein by reference in their entirety.

Unless otherwise specified, all reactions described in the examples can be conducted in an inert solvent under an inert atmosphere 4. All compounds and intermediates, unless indicated, can be purified by routine methods such as chromatography, distillation, or crystallization and stored in a stable form.

In compounds with chiral centers, the R, S, and racemic mixtures are to be considered within the scope of the present invention unless otherwise specified or unless specified in references that relate to the starting materials or known components.

Equivalents

Those skilled in the arts can recognize or be able to ascertain, using no more then routine experimentation, many equivalents to the inventions, materials, methods, and components described herein. Such equivalents are intended to be within the scope of the claims of this patent.

While this invention has been particularly shown and described with references to preferred embodiments thereof, it will be understood by those skilled in the art that various changes in form and details may be made therein without departing from the scope of the invention encompassed by the appended claims.

### EXAMPLES

### Example 1

Compound 1 is an example of a Compound 1 type molecule. The compound has targeting ligands that can bind with high affinity to prostate specific membrane antigen (PSMA) and to sigma receptors. Both of these targets are highly overexpressed on the surface of prostate cancer cells. In addition the compound has a masked female adaptor comprised of a trimer of lys-d-Ala-d-Ala, that can be unmasked by plasmin. Activated plasmin is present on the surface of tumor cells. When unmasked the d-Ala-d-Ala trimer can bind essentially irreversibly (with Kd of approximately 10ˆ-17M.) to a trimer of vancomycin a on Compound 2 of the structure shown in Example 2.

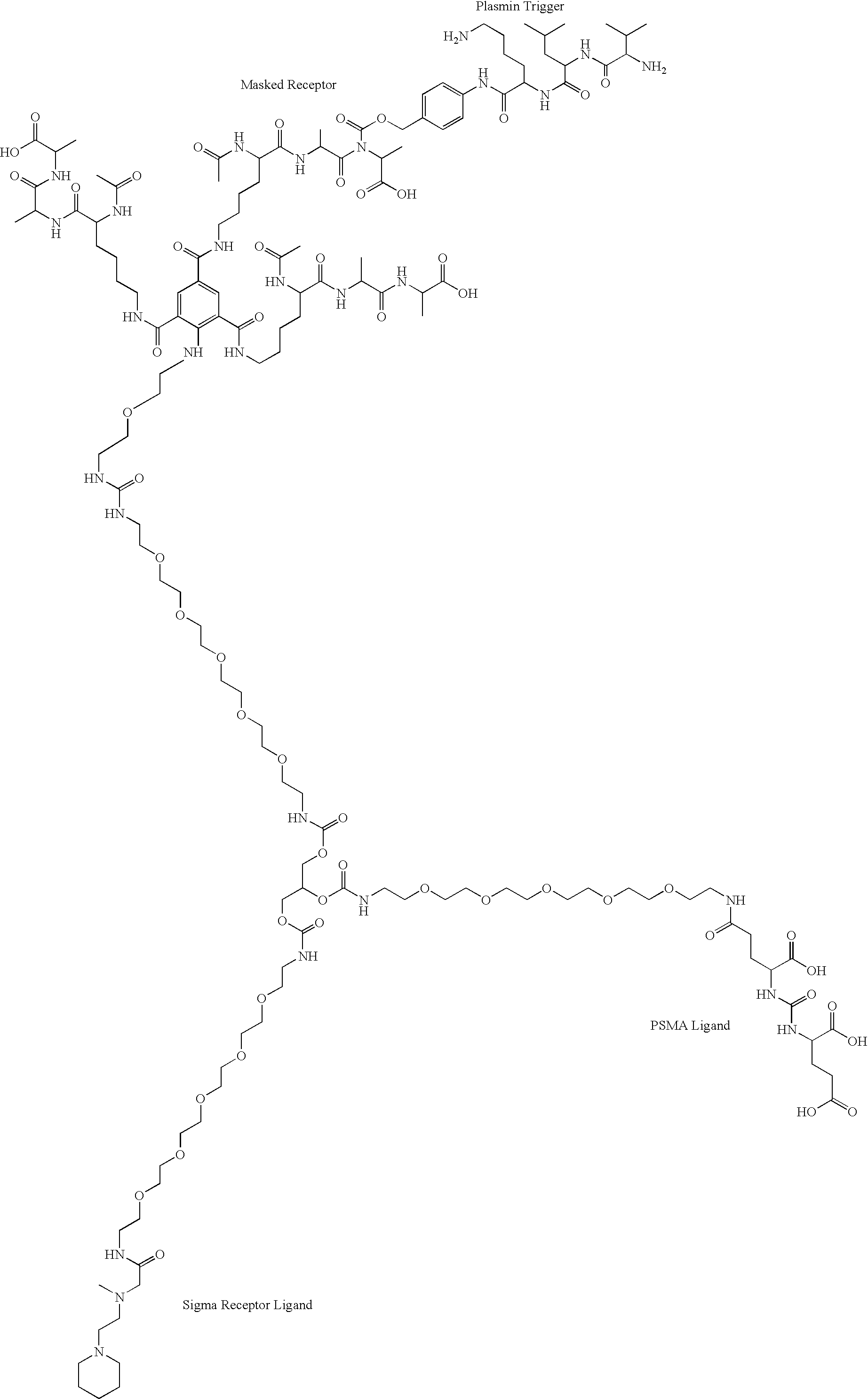

### Example 2

Example 2 is a compound that can deliver in conjunction with Compound 1 the cytotoxic agent indanocine to prostate cancer cells that express the targeting pattern comprised of PSMA and sigma receptors and plasmin. The compound has indanocine coupled by an intracellular trigger that can be activated preferentially inside cells upon conversion of the disulfide to a thiol group. Compound 2 has a trimer of vacomycin attached to the linker system. This trimer can bind to the d-Ala-d-ala trimer on a molecule of Compound 1 on the tumor cell surface. Tumor associated plasmin can than unmask the protected d-Ala-d-ala groups of Compound 2. These unmasked groups can in turn bind to 2 additional molecules of Compound 2. Repetition of this process can lead to an exponential increase in the quantity of Compound 2 bound to the tumor surface. The complex can eventually be internalized by receptor mediated endocytosis. whereupon the indanocine can be liberated and kill the tumor cell. 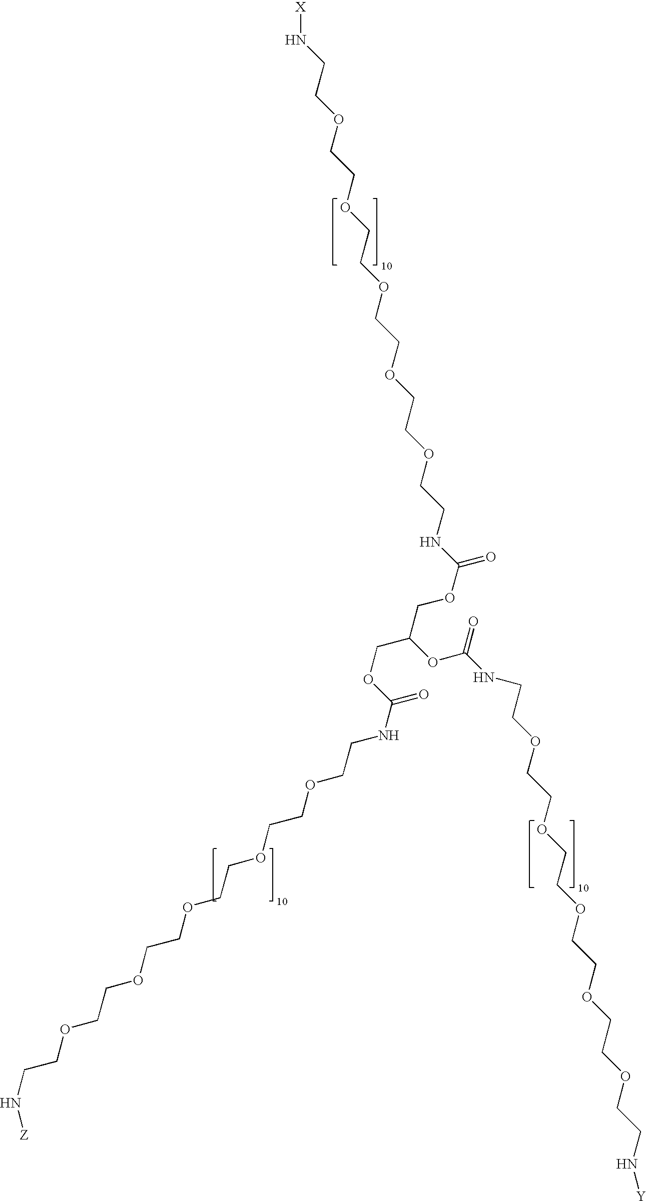 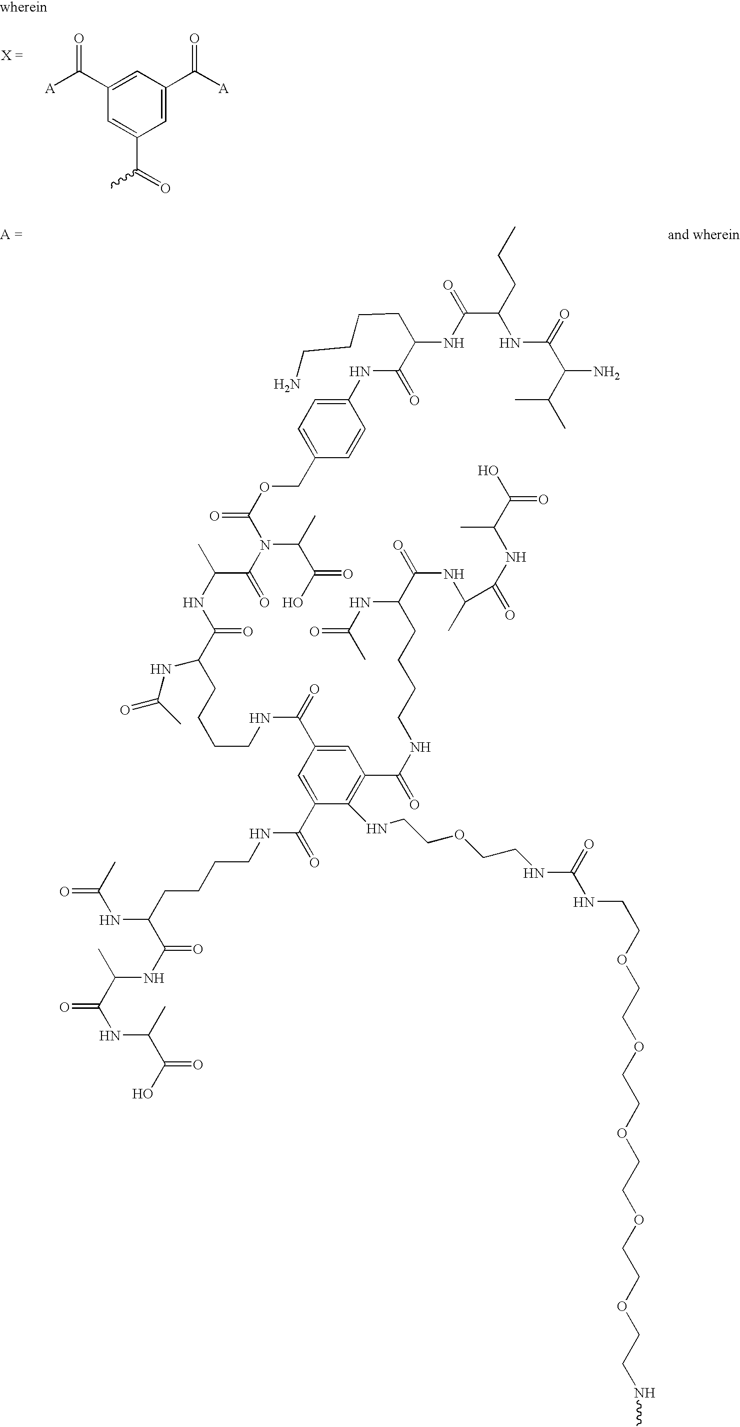 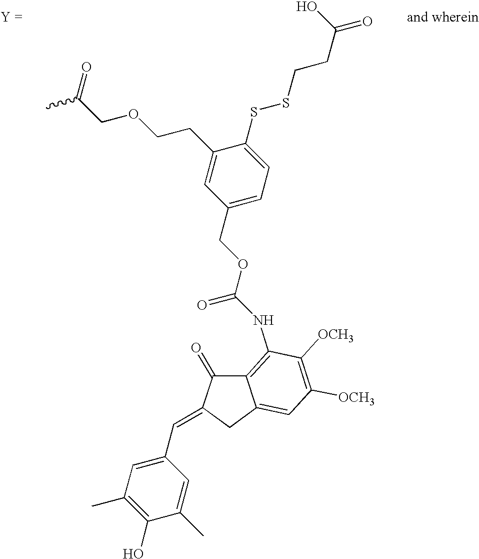 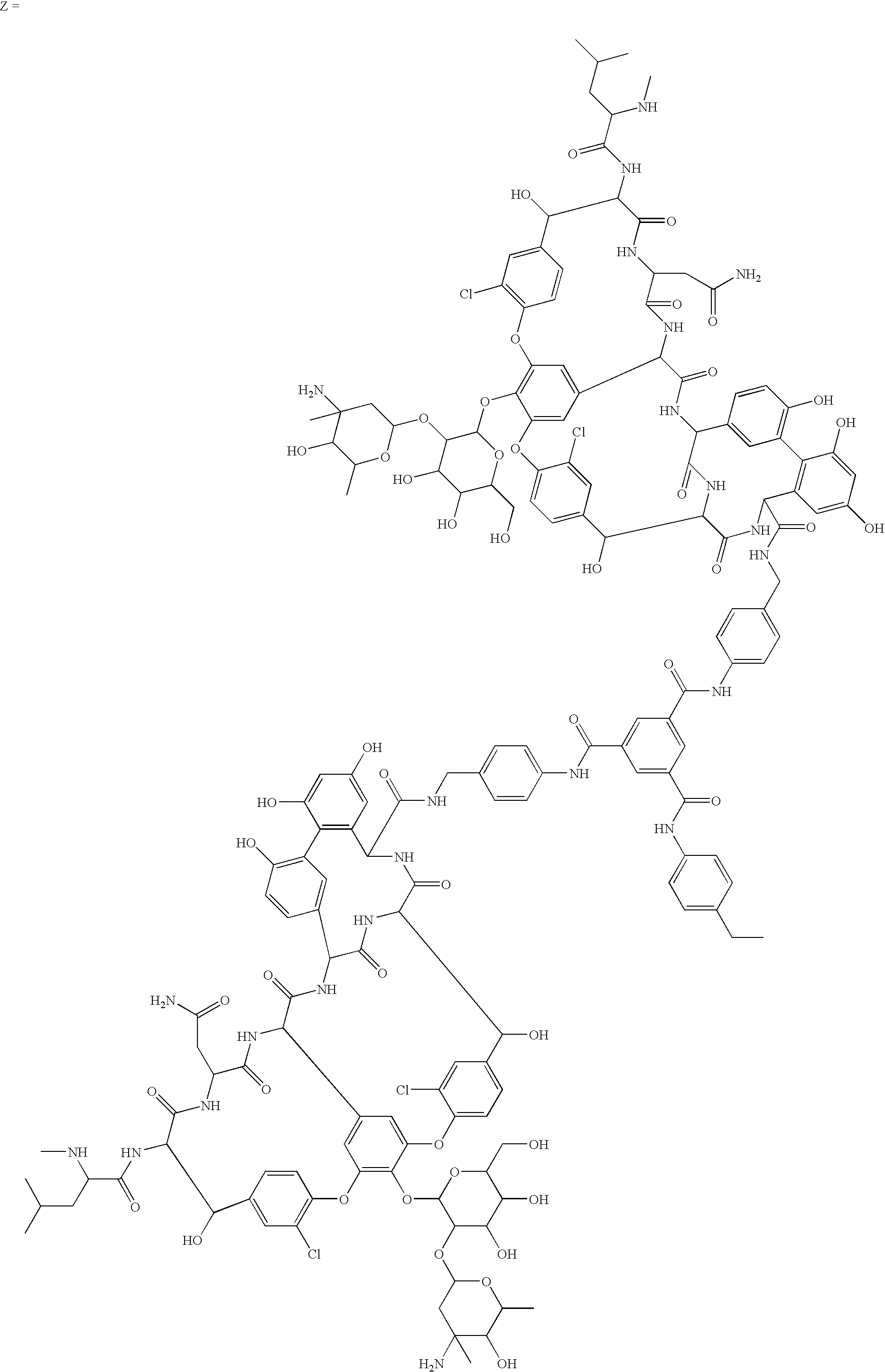 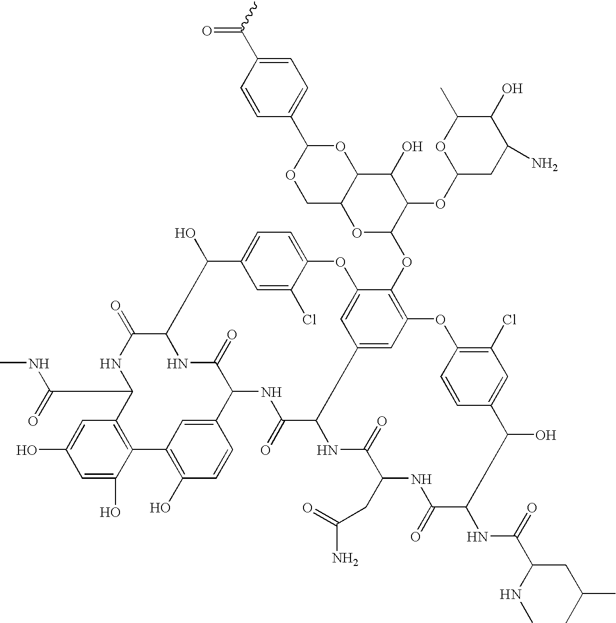
and wherein the wavy lines are the respective sites of connection. The stereochemistry for the components is as described previously or previously referenced.

### Example 3

Compound 3 is similar to Compound 1 but also has an ouabain group to anchor the complex to the Na/K ATPase and thereby retard endocytosis allowing increased time for amplification to occur.

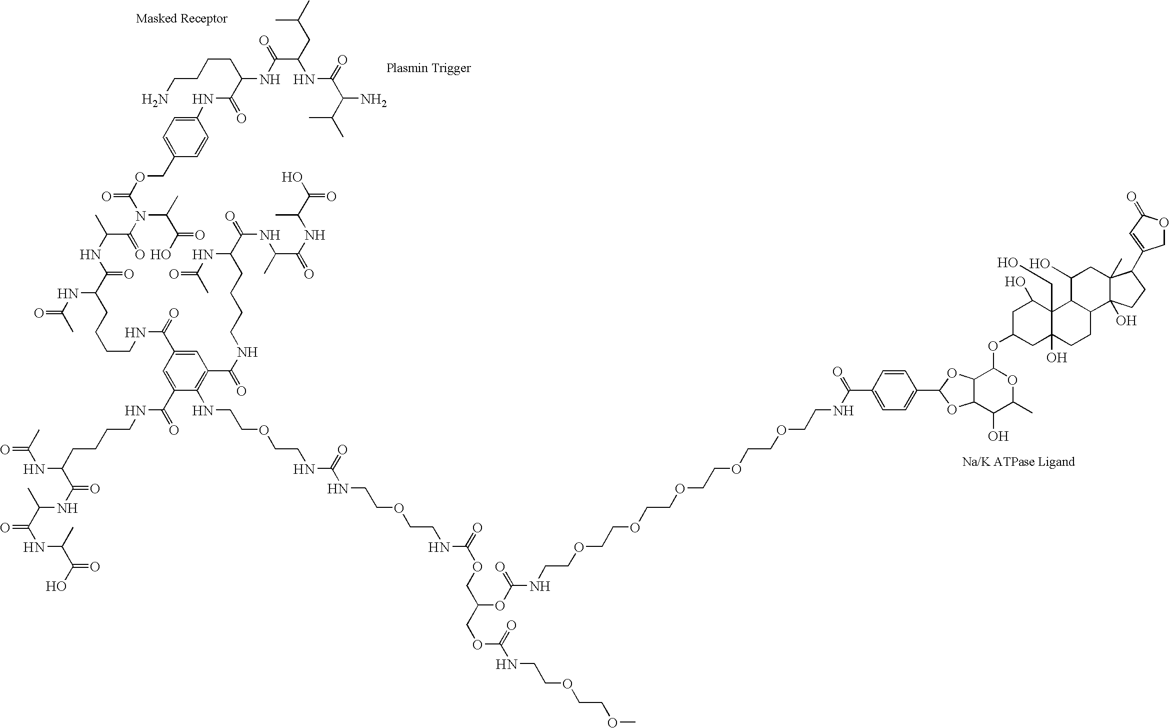

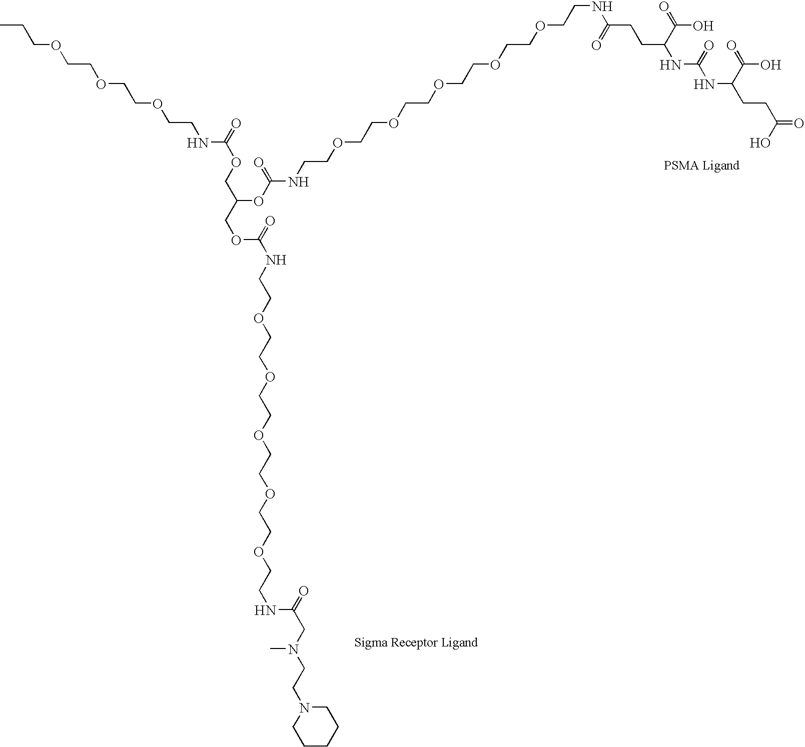

### Example 4

Example 4 demonstrates a targeting ligand for prostate specific membrane antigen. Compound 8 was synthesized and was found to be a potent inhibitor of PSMA with an IC50=8 nM.

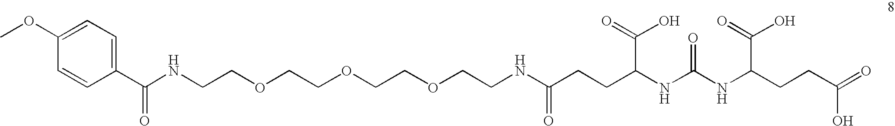

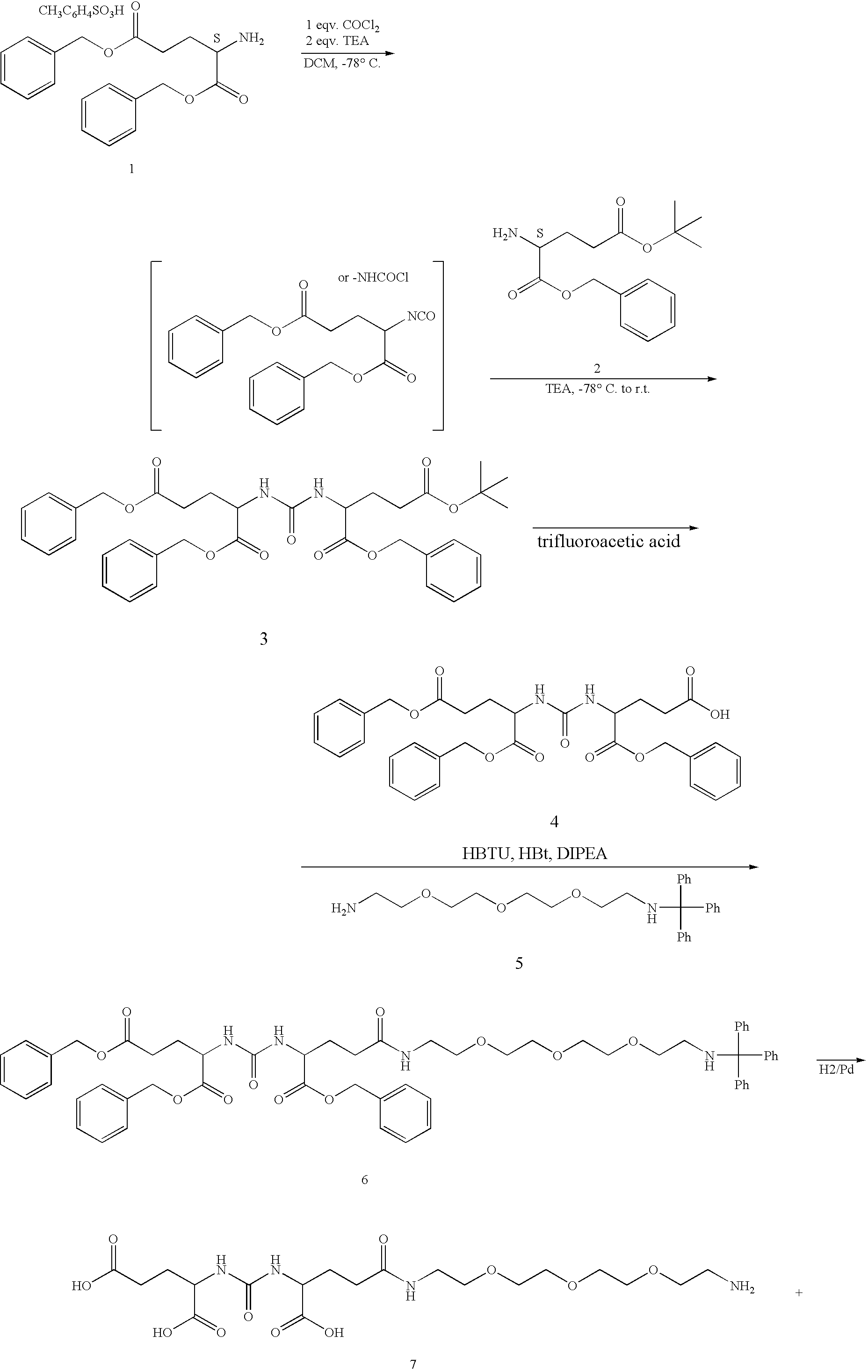

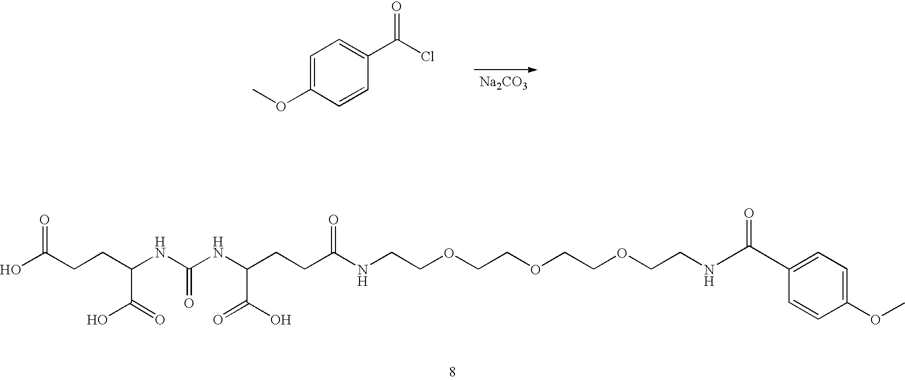

Compound 8 was synthesized by the following route.

Compound 1 was treated with 1 equivalent of phosgene and 2 equivalents of triethylamine in dichloromethane at −78 C. Then compound 2 was added along with 2 equivalents of triethylamine. The reaction was allowed to warm to room temperature and stirred overnight. Compound 3 was isolated by silica chromatography. Treatment with trifluoracetic acid in dichloromethane gave compound 4. Compound 5 was then coupled with Compound 4 using 1.2 equivalents of HBTU, 2.2 equivalents of diisopropylethylamine, and 1 equivalent of hydoxybenzotriazole in dimethylformamide. The product, Compound 6 was isolated by silica chromatography and deprotected by hydrogenation at atmospheric pressure. with Pd on carbon in methanol. The product, compound 7 was reacted with p-methoxybenzoyl chloride in with sodium carbonate as base in water to yield compound 8. Compound 8 was purified by reverse phase HPLC. All compounds were compatible with their assigned structures by proton NMR. The structure of Compound 6 was also confirmed by C¹³ NMR and mass spectroscopy.

The ability of Compound 8 to inhibit the enzymatic activity of PSMA (and consequently to bind to the enzyme) was evaluated using the method described previously in Ser. No. 09/712,465 Nov. 15, 2000 Glazier, Arnold. “Selective Cellular Targeting: Multifunctional Delivery Vehicles, Multifunctional Prodrugs, Use as Neoplastic Drugs. The IC50 for Compound 8 was 8 nanomolar.

1. A method for the site specific delivery to a target of effector molecules in vitro or in vivo; wherein said method is comprised of contacting the target with Compound 1 and Compound 2; and
wherein Compound 1 is comprised of at least one group that can bind to the target, and at least one masked female adaptor; and
wherein Compound 2 is comprised of at least one male ligand; at least one masked female adaptor; and at least one effector group; and
wherein the masked female adaptors cannot bind to the male ligands; and wherein the masked female adaptors can be unmasked by the action of an enzyme or other biomolecule at the target site to yield female adaptors; and wherein each female adaptor can bind to at least one male ligand; and each male adaptor can bind to at least one female adaptor; and
wherein the effector group is a group that directly or indirectly exerts an activity at the target.

2. A method of claim 1 wherein Compound 2 is comprised of at least two masked female adaptors.

3. A method of claim 1 wherein Compound 1 is comprised of the groups:
{T and [pF]_{q}}
wherein T is a targeting agent that can bind to R; wherein R is a receptor at the target; and wherein each pF is independently a masked female adaptor; and
wherein q is an integer between 1 and about 200; and wherein the groups pF can be the same or different;
and wherein Compound 2 is comprised of:
{[M]ₘ and [E]ₒ and [pF]ₙ}
wherein M is a male ligand; E is an effector group; and wherein the groups M can be the same or different; and wherein the groups E can be the same or different; and wherein the groups pF can be the same or different; and wherein o is an integer between 1 and about 10; and m is an integer between 1 and about 200; and n is an integer between 1 and about 200;
and wherein the group pF can be unmasked by at least one triggering enzyme at the target.

4. A method of claim 3 in which q=1; m=1; o=1; and n=2.

5. A method of claim 3 wherein the triggering enzyme is enriched at the target.

6. A method of claim 3 wherein either R, or the triggering enzyme, or both, are enriched at the target compared to at a non-target.

7. A method of claim 6 wherein Compound 1 has the following structure:
T-L-PF
and wherein Compound 2 has the structure: 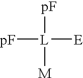
and wherein L is a linker.

8. A method of claim 7 wherein the target is a tumor.

9. A method of claim 7 in which the target is a tumor or both the tumor and the tissue of tumor origin.

10-13. (canceled)

14. A compound; wherein said compound is a prodrug that can undergo biotransformation into a drug; wherein said drug gains the ability to selectively bind at least one additional molecule of the prodrug; and wherein bound prodrug can undergo biotransformation into the drug which can selectively bind additional molecules of the prodrug.

15. A compound of claim 14 that can undergo biotransformation into a drug; wherein said drug can bind at least two molecules of the prodrug.

16. A compound of claim 15 comprised of at least one male ligand; at least one masked female adaptor; and at least one effector group; and
wherein the masked female adaptors cannot bind to the male ligands; and wherein the masked female adaptors can be unmasked by the action of a triggering enzyme or other biomolecules to yield female adaptors; and wherein each female adaptor can bind to at least one male ligand; and each male adaptor can bind to at least one female adaptor; and
wherein the effector group is a group that directly or indirectly exerts an activity at the target.

17. A compound of claim 16 comprised of:
{[M]ₘ and [E]ₒ and [PF]ₙ}
wherein M is a male ligand; E is an effector group; and wherein the groups M can be the same or different; and wherein the groups E can be the same or different; and wherein the groups pF can be the same or different; and wherein o is an integer between 1 and about 10; and m is an integer between 1 and about 200; and n is an integer between 1 and about 200.

18. A compound of claim 17 with the following structure: 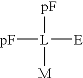
and wherein L is a linker.

19-29. (canceled)

30. A prodrug that can undergo biotransformation into a drug wherein said drug gains the ability to selectively bind to at least one molecule of a second type of drug compound.

31. A compound of claim 30 wherein the prodrug is comprised of a targeting agent that can bind to a target receptor; and at least one masked female adaptors; wherein the masked female adaptors cannot bind to the male ligands; and wherein the masked female adaptors can be unmasked by the action of a triggering enzyme to yield female adaptors; and wherein each female adaptor can bind to at least one male ligand; and each male adaptor can bind to at least one female adaptor; and wherein the male adaptors are groups present on the second type of drug compound.

32. A compound of claim 31 comprised of the groups:
{T and [pF]_{q}}
wherein T is a targeting agent that can bind to R; wherein R is a receptor at the target; and wherein each pF is independently a masked female adaptor; and wherein q is an integer between 1 and about 200; and wherein the groups pF can be the same or different.

33. A compound of claim 32 wherein T is tumor selective.

34. A compound of claim 33 wherein T can bind to a receptor selected from the following group: Prostate Specific Membrane Antigen; Somatostatin receptors; Luteinizing releasing hormone receptor; Bombesin/gastrin releasing peptide receptor; Sigma receptor; STEAP antigen; Prostate Stem Cell Antigen; Platelet Derived Growth Factor alpha receptor; Hepsin; PATE; Gonadotropin-Releasing Hormone receptor;
Transmembrane serine protease (TMPRSS2); tissue factor; c-Met; Urokinase; Urokinase receptor; MMP-1, MMP-2, MMP-7, MMP-9; and MMP-14.

35. A compound of claim 32 with the structure: 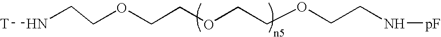
wherein n5 is an integer between 0 and about 200.

36.-43. (canceled)
